(19) United States
(12) Patent Application Publication
(10) Pub. No.: US 2020/0131156 A1
(43) Pub. Date: Apr. 30, 2020

(54) ARYLSULFONYLPYROLECARBOXAMIDE DERIVATIVES AS Kv3 POTASSIUM CHANNEL ACTIVATORS

(71) Applicant: H. Lundbeck A/S, Valby (DK)

(72) Inventors: Anette Graven SAMS, Valby (DK); Lars Kyhn RASMUSSEN, Valby (DK); Wanwan YU, Valby (DK); Paul Robert FLEMING, Cambridge (GB)

(21) Appl. No.: 16/667,563

(22) Filed: Oct. 29, 2019

(30) Foreign Application Priority Data
Oct. 30, 2018 (DK) PA201800787

(51) Int. Cl. C07D 403/12; C07D 401/12; C07D 487/04; C07D 413/12; C07D 417/12; C07D 405/14

(52) U.S. Cl. CPC C07D 403/12; C07D 401/12; C07D 405/14; C07D 413/12; C07D 417/12; C07D 487/04

(57) ABSTRACT

The present invention provides novel compounds which activate the Kv3 potassium channels. The compounds have the structure

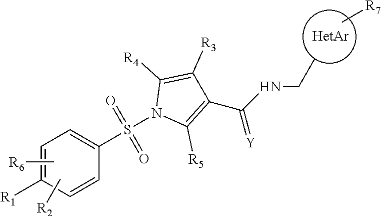

Separate aspects of the invention are directed to pharmaceutical compositions comprising said compounds and use of the compounds to treat disorders responsive to the activation of Kv3 potassium channels.

This application claims priority to Denmark Application No. PA 2018 00787, filed Oct. 30, 2018, the content of which is hereby incorporated by reference in its entirety.

### FIELD OF THE INVENTION

The present invention relates to novel compounds which activate the Kv3 potassium channels. Separate aspects of the invention are directed to pharmaceutical compositions comprising said compounds and uses of the compounds as a medicament.

### BACKGROUND OF THE INVENTION

Voltage-dependent potassium (Kv) channels conduct potassium ions (K⁺) across cell membranes in response to changes in the membrane potential and can thereby regulate cellular excitability by modulating (increasing or decreasing) the electrical activity of the cell. Functional Kv channels exist as multimeric structures formed by the association of four alpha and four beta subunits. The alpha subunits comprise six transmembrane domains, a pore-forming loop and a voltage-sensor, and are arranged symmetrically around a central pore. The beta or auxiliary subunits interact with the alpha subunits and can modify the properties of the channel complex to include, but not be limited to, alterations in the channel's electrophysiological or biophysical properties, expression levels or expression patterns. Nine Kv channel alpha subunit families have been identified and are termed Kv1 through Kv9. As such, there is an enormous diversity in Kv channel function that arises as a consequence of the multiplicity of sub-families, the formation of both homomeric and heteromeric subunits within sub-families and the additional effects of association with beta subunits (Christie, 25 Clinical and Experimental Pharmacology and Physiology, 1995, 22, 944-951).

The Kv3 channel family consists of Kv3.1 (encoded by the KCNC1 gene) and Kv3.2 (encoded by the KCNC2 gene), Kv3.3 (encoded by the KCNC3 gene) and Kv3.4 (encoded by the KCNC4 gene) (Rudy and McBain, 2001). Kv3.1, Kv3.2 and Kv3.3 are prominently expressed in the central nervous system (CNS) whereas Kv3.4 expression pattern also included peripheral nervous system (PNS) and skeletal muscle (Weiser et al. 1994). Although Kv3.1, Kv3.2 and Kv3.3 channels are broadly distributed in the brain (cerebellum, globus pallidus, subthalamic nucleus, thalamus, auditory brain stem, cortex and hippocampus), their expression is restricted to neuronal populations able to fire action potential (AP) of brief duration and to maintain high firing rates such as fast-spiking inhibitory interneurons (Rudy and McBain, 2001). Consequently, Kv3 channels display unique biophysical properties distinguishing them from other voltage-dependent potassium channels. Kv3 channels begin to open at relatively high membrane potentials (more positive than −20 mV) and exhibit very rapid activation and deactivation kinetics (Kazmareck and Zhang, 2017). These characteristics ensure a fast repolarization and minimize the duration of after-hyperpolarization required for high frequency firing without affecting subsequent AP initiation and height.

Among Kv3 channels, Kv3.1 and Kv3.2 are particularly enriched in gabaergic interneurons including parvalbumin (PV) and somatostatin interneurons (SST) (Chow et al., 1999). Genetic ablation of Kv3.2 has been shown to broaden AP and to alter the ability to fire at high frequency in this neuronal population (Lau et al., 2000). Further, this genetic manipulation increased susceptibility to seizures. Similar phenotype was observed in mice lacking Kv3.1 and Kv3.3 confirming a crucial role of these channels in excitatory/inhibitory balance observed in epilepsy. This was confirmed at clinical level since several mutations within the KCNC1 (Kv3.1) gene have been shown to cause rare forms of epilepsy in human (Muona et al., 2015; Oliver et al., 2017). Consequently, positive modulators of Kv3 channel activators might restore excitatory/inhibitory imbalance, associated with epilepsy, through increasing the activity of inhibitory interneuron.

In addition to seizure susceptibility, excitatory/inhibitory imbalance has been postulated to participate in cognitive dysfunctions observed in a broad number of psychiatric disorders, including schizophrenia and autism spectrum disorder (Foss-Feig et al., 2017) as well as bipolar disorder, ADHD (Edden et al., 2012), anxiety-related disorders (Fuchs et al., 2017), and depression (Klempan et al., 2009). Post-mortem studies revealed alterations of the certain gabaergic molecular markers in patients suffering from these pathologies (Straub et al., 2007; Lin and Sibille, 2013). Importantly, inhibition from parvalbumin and somatostatin interneurons projecting to the pyramidal excitatory neurons is essential for the synchronized oscillatory activity of neural network, such as gamma oscillations (Bartos et al., 2007; Veit et al., 2017). This last type oscillation regulates diverse cognitive processes from sensory integration, attention, working memory and cognitive flexibility, domains that are particularly affected in psychiatric disorders (Herrmann and Demiralp; 2005). Therefore, Kv3 channel activators might rescue cognitive dysfunction and their associated alteration in gamma oscillations by increasing interneuron functions.

Both epileptiform activities and alterations of oscillations in the range of gamma have been observed at preclinical as well as clinical level in Alzheimer's disease (Palop and Mucke, 2016). While there is no current evidence of Kv3 channel alterations in Alzheimer's disease, Kv3 activators, through their actions on interneurons, could relieve both network alterations and cognitive abnormalities observed in this pathology and other neurodegenerative disorders.

Kv3.1 channels are particularly enriched in auditory brain stem. This particular neuronal population is required to fire AP at high rate (up to 600 Hz) and genetic ablation of Kv3.1 alters the ability of these neurons to follow high frequency stimulation (Macica et al., 2003). Kv3.1 levels in this structure has been shown to be altered in various conditions affecting auditory sensitivity, such as hearing loss (Von Hehn et al., 2004), fragile X (Strumbos et al, 2010) or tinnitus, suggesting that Kv3 activators might have therapeutic potential in these disorders.

Kv3.4 channels and, to a lesser extent, Kv3.1 are expressed in the dorsal root ganglion (Tsantoulas and McMahon, 2014). Hypersensitivity to noxious stimuli in animal models of chronic pain have been associated with AP broadening (Chien et al., 2007). This phenomenon is partially due to alteration of Kv3.4 expression and function supporting the rationale to use Kv3 channels activator in the treatment of certain chronic pain conditions.

Kv3.1 and Kv3.2 are widely distributed within suprachiasmic nucleus, a structure responsible for controlling circadian rhythms. Mice lacking both Kv3.1 and Kv3.2 exhibit fragmented and altered circadian rhythm (Kudo et al., 2011). Consequently, Kv3.1 channel activators might be relevant for the treatment of sleep and circadian disorders, as well as sleep disruption as core symptom of psychiatric and neurodegenerative disorders.

KV3.1 channels are highly expressed in parvalbumin-positive interneurons located in the striatum (Munoz-Manchado et al., 2018). Although numerically rare compared to other neuronal populations of the striatum, they strongly influence striatal activity and consequently motoric function. Pharmacological inhibition of this population elicited dyskinetic movement, confirming their key role in motoric regulation and eventually in the pathophysiology of movement disorders (Gittis et al., 2011). Indeed, striatal parvalbumin interneuron alterations at both functional and density levels have been reported in numerous movement disorders including Huntington's disease (Lallani et al., 2019; Reiner et al., 2013), L-dopa-induced dyskinesia (Alberico et al., 2017), obsessive compulsive disorders (Burguiere et al., 2013), Tourette syndrome (Kalanithi et al., 2005; Kataoka et al., 2010). Consequently, positive modulator of KV3 channels could exert attenuate abnormal movement observed in these pathologies through the modulation of striatal parvalbumin interneurons.

Autifony Therapeutics is developing AUT-00206 (AUT-6; AUT-002006), a Kv3 subfamily voltage-gated potassium channel modulator, for the potential oral treatment of schizophrenia and fragile X. Autifony is also developing another Kv3 subfamily voltage-gated potassium channel modulator, AUT-00063, for the potential treatment of hearing disorders, including noise-induced hearing loss. The compounds are disclosed in WO2017103604 and WO2018020263.

Although patients suffering from the above-mentioned disorders may have available treatment options, many of these options lack the desired efficacy and are accompanied by undesired side effects. Therefore, an unmet need exists for novel therapies for the treatment of said disorders.

In an attempt to identify new therapies, the inventors have identified a series of novel compounds as represented by Formula I which act as Kv3 channel activators, in particular as Kv3.1 channel activators. Accordingly, the present invention provides novel compounds as medicaments for the treatment of disorders which are modulated by the potassium channels.

### SUMMARY OF THE INVENTION

The present invention relates to a compound of Formula I (hereinafter also referred to as Compound (I))

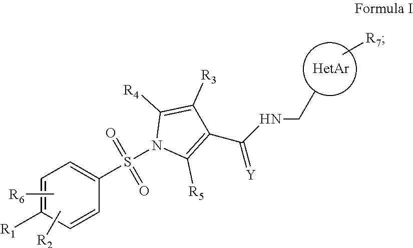

wherein
R1 is selected from the group consisting of H, C₁-C₄ alkyl, C₁-C₄ fluoroalkyl, C₁-C₄ alkoxy, C₁-C₄ fluoroalkoxy, C₃-C₈ cycloalkyl, C₁-C₄ thioalkyl, C₁-C₄ thiofluoroalkyl and halogen, such as fluorine and chlorine;
R2 and R6 are independently selected from the group consisting of H, C₁-C₄ alkyl, C₁-C₄ alkoxy, and halogen, such as fluorine and chlorine;
R3 is selected from the group consisting of H, fluorine and C₁-C₄ alkyl;
R4 and R5 are selected from the group consisting of H and fluorine;
R7 is selected from the group consisting of H, C₁-C₄ alkyl, halogen, such as fluorine and chlorine, C₁-C₄ alkoxy, fluoroalkyl, fluoroalkoxy and C₁-C₄ alkylamino;
Y is selected from the group consisting of oxygen and sulfur; and
HetAr is selected from the group consisting of 5-membered heteroaryl, 6-membered heteroaryl, and a bicyclic heteroaromatic ring system and HetAr may be substituted with one or more independently selected R7 substituents;
wherein when R1 is C₁-C₄ alkoxy, in particular methoxy, it may form a ring closure with R2 or R6 when any one of these is C₁-C₄ alkyl, in particular methyl;
   or pharmaceutically acceptable salts of Compound (I).

The invention also concerns a pharmaceutical composition comprising a compound according to the invention and a pharmaceutically acceptable excipient.

Furthermore, the invention concerns Compound (I) for use as a medicament.

Further, the invention concerns use of Compound (I) for the treatment or alleviation of epilepsy, schizophrenia, in particular cognitive impairment associated with schizophrenia (CIAS), autism spectrum disorder, bipolar disorder, ADHD, anxiety-related disorders, depression, cognitive dysfunction, Alzheimer's disease, fragile X syndrome, chronic pain, hearing loss, sleep and circadian disorders, sleep disruption and movement disorders, such as Huntington's disease, L-dopa-induced dyskinesia, obsessive compulsive disorders, and Tourette syndrome.

Certain aspects of the present invention were made with assistance of financial support from the Innovative Medicines Initiative, Grant Agreement Number: 115489.

### BRIEF DESCRIPTION OF FIGURES

FIG. 1A: Effect of Compound 86 on the Kv3.x family of channels. Upper panel, concentration dependent hyperpolarizing shift in activation threshold. Lower panel, concentration dependent increase in current amplitude measured at the −10 mV step of the IV curve. Dashed lines indicates the 5 mV or 30% increase potency measure point.

FIG. 1B: Effect of Compound 90 on the Kv3.x family of channels. Upper panel, concentration dependent hyperpolarizing shift in activation threshold. Lower panel, concentration dependent increase in current amplitude measured at the −10 mV step of the IV curve. Dashed lines indicates the 5 mV or 30% increase potency measure point.

FIG. 2A: Electrophysiological brain slice recordings. Compound 90 increases the outward K+ current recorded from FSI. Outward currents elicited by stepping the voltage to 0 mV. Recordings were conducted before (Control) or in the presence of 10 μM Compound 90. The compound-mediated increase in current was largely reversible (Wash).

FIG. 2B: Electrophysiological brain slice recordings. Compound 90 increases the outward K+ current recorded from FSI. Current recorded at 0 mV as a function of time. Compound 90 (10 μM) was applied to the perfusate as indicated by the bar.

FIG. 2C: Electrophysiological brain slice recordings. Compound 90 increases the outward K+ current recorded from FSI. Outward current in presence of Compound 90 (10 μM) relative to baseline. Compound 90 increased the current by close to 50% (144±4%, n=7, baseline 100%).

FIG. 2D: Outward current in presence of Compound 86 (10 μM) relative to baseline. Data were obtained from similar experiments as those summarized in FIGS. 2A-C. Compound 86 (10 μM) increased the outward current to 121±2% of the baseline level (n=6). Note that the relative contribution of Kv3 channels to the total current level in these experiments is unclear. Neither of the two selected compounds had any significant effect on the outward current from PYR cells (not shown).

FIG. 3A: Electrophysiological brain slice recordings. Compound 90 increases FSI excitability at low concentrations (0.1 and 1 μM) and decreases excitability at higher concentrations (10 μM). Open circles: low input current (5-10 APs before compound application), Closed circles: high input current (15-20 APs before compound application). APs elicited by 800 ms-long square current injections in the absence (Baseline) or the presence of increasing (accumulating) concentrations of Compound 90. The holding potential was set at −70 mV. The size of the current injections was chosen to elicit 5-10 (low input current) and 15-20 (high input current) APs under baseline, respectively.

FIG. 3B: Electrophysiological brain slice recordings. Compound 90 increases FSI excitability at low concentrations (0.1 and 1 μM) and decreases excitability at higher concentrations (10 μM). Open circles: low input current (5-10 APs before compound application), Closed circles: high input current (15-20 APs before compound application). Number of APs as a function of time elicited by low (white circles) or high (gray circles) input currents, respectively. Following a stable baseline, Compound 90 was applied at increasing concentrations (15 min at each concentration) as shown by the bar. There was an increase in FSI excitability at 0.3 and 1 μM whereas at 10 μM, the excitability decreased, reaching a level below baseline (n=6).

FIG. 3C: Electrophysiological brain slice recordings. Compound 90 increases FSI excitability at low concentrations (0.1 and 1 μM) and decreases excitability at higher concentrations (10 μM). Open circles: low input current (5-10 APs before compound application), Closed circles: high input current (15-20 APs before compound application). Similar data as those summarized in panel B, but with Compound 86 applied at increasing concentrations. Note that Compound 86 increased excitability at 0.3 and 1 μM, whereas a slight reduction in excitability was observed at 10 μM (when compared to data at 1 μM (n=7).

FIG. 4A: In vivo pharmacokinetic time profile of Compound 90 in rats at 3 and 30 mg/kg PO.

FIG. 4B: In vivo pharmacokinetic time profile of Compound 90 in rats at 3 and 10 mg/kg SC.

FIG. 5A: In vivo pharmacokinetic time profile of Compound 90 in mice at 3 and 30 mg/kg PO.

FIG. 5B: In vivo pharmacokinetic time profile of Compound 90 in mice at 3 and 10 mg/kg SC.

FIG. 6: In vivo pharmacokinetic time profile of Compound 86 in rats.

FIG. 7: In vivo pharmacokinetic time profile of Compound 86 in mice.

### DETAILED DESCRIPTION OF THE INVENTION

The invention is described in further detail below, first in general and then in more detail in the embodiments of the invention and the following Experimental Section.

The present invention provides novel compounds that may be useful as medicaments for the treatment of disorders which are modulated by the potassium channels. The compounds of the invention have the generalized structure of Formula I:

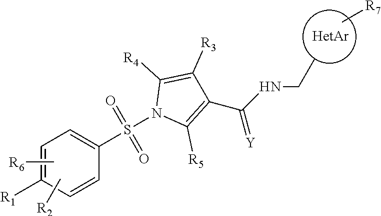

wherein R1 to R7 and HetAr are selected as disclosed above and in the more particular embodiments below.

According to a specific embodiment of the invention the compound is selected from a group of compounds as described below.

Reference to compounds encompassed by the present invention includes racemic and chiral mixtures of the compounds, optically pure isomers of the compounds for which this is relevant as well as tautomeric forms the compounds for which this is relevant.

Furthermore, the invention includes compounds in which one or more hydrogen has been exchanged by deuterium.

Furthermore, the compounds of the present invention may potentially exist as polymorphic and amorphic forms and in unsolvated as well as in solvated forms with pharmaceutically acceptable solvents such as water and ethanol. Both solvated and unsolvated forms of the compounds are encompassed by the present invention.

The compound according to the invention may be in a pharmaceutical composition comprising the compound and a pharmaceutically acceptable excipient.

In one embodiment, the invention relates to a compound according to the invention for use in therapy.

In another embodiment, the invention relates to a method of treating a patient in the need thereof suffering from epilepsy, schizophrenia, schizoaffective disorder, cognitive impairment associated with schizophrenia, bipolar disorder, ADHD, anxiety, depression, cognitive dysfunction, Alzheimer's disease, hearing loss, tinnitus, fragile X syndrome, pain, sleep disorder and circandian disorders, sleep disruption and movement disorders, such as Huntington's disease, L-dopa-induced dyskinesia, obsessive compulsive disorders, and Tourette syndrome, comprising administering to the subject a therapeutically effective amount of a compound according to the invention.

According to an embodiment the compounds of the invention are for use as a medicament. In a particular embodiment, the compounds of the invention are for use in treating or alleviating epilepsy, schizophrenia, schizoaffective disorder, cognitive impairment associated with schizophrenia, bipolar disorder, ADHD, anxiety, depression, cognitive dysfunction, Alzheimer's disease, hearing loss, tinnitus, fragile x syndrome, pain, sleep disorder and circandian disorders, sleep disruption and movement disorders, such as Huntington's disease, L-dopa-induced dyskinesia, obsessive compulsive disorders, and Tourette syndrome.

In another embodiment, the compound of the invention is for the manufacture of a medicament for the treatment of epilepsy, schizophrenia, schizoaffective disorder, cognitive impairment associated with schizophrenia, bipolar disorder, ADHD, anxiety, depression, cognitive dysfunction, Alzheimer's disease, hearing loss, tinnitus, fragile x syndrome, pain, sleep disorder, circandian disorders, sleep disruption and movement disorders, such as Huntington's disease, L-dopa-induced dyskinesia, obsessive compulsive disorders, and Tourette syndrome.

### Substituents

In the present context, “optionally substituted” means that the indicated moiety may or may not be substituted, and when substituted is mono- or di-substituted. It is understood that where no substituents are indicated for an “optionally substituted” moiety, then the position is held by a hydrogen atom.

The notation R1, R2, R3, R5, R6 and R7 may be used interchangeably with the notation R₁, R₂, R₃, R₄, R₅, R₆, and R₇.

A given range may interchangeably be indicated with “-” (dash) or “to”, e.g., the term “C₁₋₄ alkyl” is equivalent to “C₁ to C₄ alkyl”.

The term “C₁₋₄ alkyl” refer to an unbranched or branched saturated hydrocarbon having from one up to four carbon atoms, inclusive. Examples of such groups include, but are not limited to, methyl, ethyl, 1-propyl, 2-propyl, 1-butyl, 2-butyl and 2-methyl-2-propyl.

The term “heteroaromatic” includes tautomeric forms of the heteroaromatic compound.

The term “C₁-C₄ alkoxy” refers to a moiety of the formula —OR, wherein R indicates C₁-C₄ alkyl as defined above. In particular, “C₁₋₄ alkoxy” refers to such moiety wherein the alkyl part has 1, 2, 3 or 4 carbon atoms. Examples of “C₁₋₄ alkoxy” include methoxy, ethoxy, n-butoxy and tert-butoxy.

The term “C₁₋₄ fluoroalkyl” refers to an alkyl having 1 to 4 carbon atoms, wherein at least one hydrogen atom is replaced with a fluorine atom, such as mono-, di-, or tri-fluoralkyl. Examples of fluoroalkyls include, but are not limited to, monofluoromethyl, difluoromethyl, trifluoromethyl, monofluoroethyl, difluoroethyl, trifluoroethyl, monofluoropropyl, difluoropropyl, trifluoropropyl, monofluorobutyl, difluorobutyl, trifluorobutyl. Preferably the fluorine atom(s) is positioned on the terminal carbon atom.

The term “C₁₋₄ fluoroalkoxy” refers to a moiety of the formula —OR_{A}, wherein R_{A} indicates C₁-C₄ fluoroalkyl as defined above. Examples of fluoroalkoxys include, but are not limited to, monofluoromethoxy, difluoromethoxy, trifluoromethoxy, monofluoroethoxy, difluoroethoxy, trifluoroethoxy, monofluoropropoxy, difluoropropoxy, trifluoropropoxy, monofluorobutoxy, difluorobutoxy, trifluorobutoxy.

The term “C₃-C₈ cycloalkyl” typically refers to cyclopropyl, cyclobutyl, cyclopentyl, cyclohexyl, cycloheptyl or cyclooctyl.

The term “C₁₋₄ thioalkyl” refers to a moiety of the formula —SR, wherein R indicates C₁-C₄ alkyl as defined above. Examples of thioalkyl include, but are not limited to, thiomethyl, thioethyl, 1-thiopropyl, 2-thiopropyl, 1-thiobutyl, 2-thiobutyl and 2-methyl-2-thiopropyl.

The term “C₁₋₄ thiofluoroalkyl” refers to a moiety of the formula —SR_{A}, wherein R_{A} indicates C₁-C₄ fluoroalkyl as defined above. Examples of thiofluoroalkyls include, but are not limited to, thiomonofluoromethyl, thiodifluoromethyl, thiotrifluoromethyl, thiomonofluoroethyl, thiodifluoroethyl, thiotrifluoroethyl, thiomonofluoropropyl, thiodifluoropropyl, thiotrifluoropropyl, thiomonofluorobutyl, thiodifluorobutyl, and thiotrifluorobutyl.

The term “heteroaryl” refers to an aromatic ring or fused aromatic rings wherein one or more ring atoms are selected from O, N or S. Examples of heteroaryls include, but are not limited to, pyrimidinyl, pyridazinyl, pyrazinyl, pyrazolyl, pyridyl, oxadiazolyl, isoxazolyl, oxazolyl, thiazolyl, imidazolyl, triazolyl, thiadiazolyl and imidazopyrimidinyl.

### Administration Routes

Pharmaceutical compositions comprising a compound of the present invention defined above, may be specifically formulated for administration by any suitable route such as the oral, rectal, nasal, buccal, sublingual, transdermal and parenteral (e.g., subcutaneous, intramuscular, and intravenous) route; the oral route being preferred.

It will be appreciated that the route will depend on the general condition and age of the subject to be treated, the nature of the condition to be treated and the active ingredient.

### Pharmaceutical Formulations and Excipients

In the following, the term, “excipient” or “pharmaceutically acceptable excipient” refers to pharmaceutical excipients including, but not limited to, fillers, antiadherents, binders, coatings, colours, disintegrants, flavours, glidants, lubricants, preservatives, sorbents, sweeteners, solvents, vehicles and adjuvants.

The present invention also provides a pharmaceutical composition comprising a compound according to the invention, such as one of the compounds disclosed in the Experimental Section herein. The present invention also provides a process for making a pharmaceutical composition comprising a compound according to the invention. The pharmaceutical compositions according to the invention may be formulated with pharmaceutically acceptable excipients in accordance with conventional techniques such as those disclosed in Remington, “The Science and Practice of Pharmacy”, 22^{nd} edition (2012), Edited by Allen, Loyd V., Jr.

In an embodiment, the present invention relates to a pharmaceutical composition comprising a compound of Formula I, such as one of the compounds disclosed in the Experimental Section herein.

Pharmaceutical compositions for oral administration include solid oral dosage forms such as tablets, capsules, powders and granules; and liquid oral dosage forms such as solutions, emulsions, suspensions and syrups as well as powders and granules to be dissolved or suspended in an appropriate liquid.

Solid oral dosage forms may be presented as discrete units (e.g., tablets or hard or soft capsules), each containing a predetermined amount of the active ingredient, and preferably one or more suitable excipients. Where appropriate, the solid dosage forms may be prepared with coatings such as enteric coatings or they may be formulated to provide modified release of the active ingredient, such as delayed or extended release, according to methods well known in the art. Where appropriate, the solid dosage form may be a dosage form disintegrating in the saliva, such as, for example, an orodispersible tablet.

Examples of excipients suitable for solid oral formulation include, but are not limited to, microcrystalline cellulose, corn starch, lactose, mannitol, povidone, croscarmellose sodium, sucrose, cyclodextrin, talcum, gelatin, pectin, magnesium stearate, stearic acid and lower alkyl ethers of cellulose. Similarly, the solid formulation may include excipients for delayed or extended release formulations known in the art, such as glyceryl monostearate or hypromellose.

If solid material is used for oral administration, the formulation may, for example, be prepared by mixing the active ingredient with solid excipients and subsequently compressing the mixture in a conventional tableting machine; or the formulation may, for example, be placed in a hard capsule, e.g., in powder, pellet or mini tablet form. The amount of solid excipient will vary widely but will typically range from about 25 mg to about 1 g per dosage unit.

Liquid oral dosage forms may be presented as, for example, elixirs, syrups, oral drops or a liquid filled capsule. Liquid oral dosage forms may also be presented as powders for a solution or suspension in an aqueous or non-aqueous liquid. Examples of excipients suitable for liquid oral formulation include, but are not limited to, ethanol, propylene glycol, glycerol, polyethylenglycols, poloxamers, sorbitol, poly-sorbate, mono- and di-glycerides, cyclodextrins, coconut oil, palm oil, and water. Liquid oral dosage forms may, for example, be prepared by dissolving or suspending the active ingredient in an aqueous or non-aqueous liquid, or by incorporating the active ingredient into an oil-in-water or water-in-oil liquid emulsion.

Further excipients may be used in solid and liquid oral formulations, such as colorings, flavorings, preservatives, etc.

Pharmaceutical compositions for parenteral administration include sterile aqueous and nonaqueous solutions, dispersions, suspensions or emulsions for injection or infusion, concentrates for injection or infusion, as well as sterile powders to be reconstituted in sterile solutions or dispersions for injection or infusion prior to use. Examples of excipients suitable for parenteral formulation include, but are not limited to water, coconut oil, palm oil and solutions of cyclodextrins. Aqueous formulations should be suitably buffered if necessary and rendered isotonic with sufficient saline or glucose.

Other types of pharmaceutical compositions include suppositories, inhalants, creams, gels, dermal patches, implants and formulations for buccal or sublingual administration.

It is requisite that the excipients used for any pharmaceutical formulation comply with the intended route of administration and are compatible with the active ingredients.

### Doses

In one embodiment, the compound of the present invention is administered in an amount from about 0.001 mg/kg body weight to about 100 mg/kg body weight per day. In particular, daily dosages may be in the range of 0.01 mg/kg body weight to about 50 mg/kg body weight per day. The exact dosages will depend upon the frequency and mode of administration, the gender, the age, the weight, and the general condition of the subject to be treated, the nature and the severity of the condition to be treated, any concomitant diseases to be treated, the desired effect of the treatment and other factors known to those skilled in the art.

A typical oral dosage for adults will be in the range of 0.1-1000 mg/day of a compound of the present invention, such as 1-500 mg/day, such as 1-100 mg/day or 1-50 mg/day. Conveniently, the compounds of the invention are administered in a unit dosage form containing said compounds in an amount of about 0.1 to 500 mg, such as 10 mg, 50 mg 100 mg, 150 mg, 200 mg or 250 mg of a compound of the present invention.

### Pharmaceutically Acceptable Salts

The compounds of this invention are generally utilized as the free substance or as a pharmaceutically acceptable salt thereof. When a compound of Formula I contains a free base such salts may be prepared in a conventional manner by treating a solution or suspension of a free base of Formula I with a molar equivalent of a pharmaceutically acceptable acid. Representative examples of suitable organic and inorganic acids are described below.

Pharmaceutically acceptable salts in the present context is intended to indicate non-toxic, i.e., physiologically acceptable salts. The term pharmaceutically acceptable salts includes salts formed with inorganic and/or organic acids such as hydrochloric acid, hydrobromic acid, phosphoric acid, nitrous acid, sulphuric acid, benzoic acid, citric acid, gluconic acid, lactic acid, maleic acid, succinic acid, tartaric acid, acetic acid, propionic acid, oxalic acid, maleic acid, fumaric acid, glutamic acid, pyroglutamic acid, salicylic acid, salicylic acid and sulfonic acids, such as methanesulfonic acid, ethanesulfonic acid, toluenesulfonic acid and benzenesulfonic acid. Some of the acids listed above are di- or tri-acids, i.e., acids containing two or three acidic hydrogens, such as phosphoric acid, sulphuric acid, fumaric acid and maleic acid. Di- and tri-acids may form 1:1, 1:2 or 1:3 (tri-acids) salts, i.e., a salt formed between two or three molecules of the compound of the present invention and one molecule of the acid.

Additional examples of useful acids and bases to form pharmaceutically acceptable salts can be found in, e.g., Stahl and Wermuth (Eds) “Handbook of Pharmaceutical Salts. Properties, Selection, and Use”, Wiley-VCH, 2008.

### Isomeric and Tautomeric Forms

When compounds of the present invention contain one or more chiral centers reference to any of the compounds will, unless otherwise specified, cover the enantiomerically or diastereomerically pure compound as well as mixtures of the enantiomers or diastereomers in any ratio.

Furthermore, some of the compounds of the present invention may exist in different tautomeric forms and it is intended that any tautomeric forms that the compounds are able to form are included within the scope of the present invention.

### Deuterated Compounds

Included in the scope of the present invention are also compounds of the invention in which one or more hydrogen has been exchanged by deuterium.

### Therapeutically Effective Amount

In the present context, the term “therapeutically effective amount” of a compound means an amount sufficient to alleviate, arrest, partly arrest, remove or delay the clinical manifestations of a given disease and its complications in a therapeutic intervention comprising the administration of said compound. An amount adequate to accomplish this is defined as “therapeutically effective amount”. Effective amounts for each purpose will depend on the severity of the disease or injury as well as the weight and general state of the subject. It will be understood that determining an appropriate dosage may be achieved using routine experimentation, by constructing a matrix of values and testing different points in the matrix, which is all within the ordinary skills of a trained physician.

### Treatment and Treating

In the present context, “treatment” or “treating” is intended to indicate the management and care of a patient for the purpose of alleviating, arresting, partly arresting, removing or delaying progress of the clinical manifestation of the disease. The patient to be treated is preferably a mammal, in particular a human being.

All references, including publications, patent applications and patents, cited herein are hereby incorporated by reference in their entirety and to the same extent as if each reference were individually and specifically indicated to be incorporated by reference and were set forth in its entirety (to the maximum extent permitted by law).

Headings and sub-headings are used herein for convenience only and should not be construed as limiting the invention in any way.

The use of any and all examples, or exemplary language (including “for instance”, “for example”, “e.g.”, and “as such”) in the present specification is intended merely to better illuminate the invention, and does not pose a limitation on the scope of invention unless otherwise indicated.

The citation and incorporation of patent documents herein is done for convenience only, and does not reflect any view of the validity, patentability and/or enforceability of such patent documents.
The present invention includes all modifications and equivalents of the subject-matter recited in the claims appended hereto, as permitted by applicable law.

### FURTHER EMBODIMENTS OF THE INVENTION

The following embodiments describe the invention in further detail. The embodiments are numbered consecutively, starting from number 1.

### EMBODIMENTS

1. A Compound (I) of Formula I

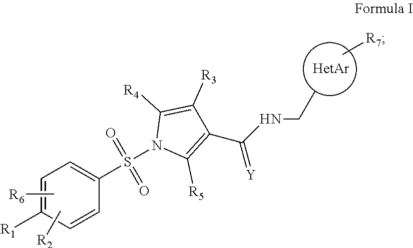

wherein
R1 is selected from the group consisting of H, C₁-C₄ alkyl, C₁-C₄ fluoroalkyl, C₁-C₄ alkoxy, C₁-C₄ fluoroalkoxy, C₃-C₈ cycloalkyl, C₁-C₄ thioalkyl, C₁-C₄ thiofluoroalkyl and halogen, such as fluorine and chlorine;
R2 and R6 are independently selected from the group consisting of H, C₁-C₄ alkyl, C₁-C₄ alkoxy, and halogen, such as fluorine and chlorine;
R3 is selected from the group consisting of H, fluorine and C₁-C₄ alkyl;
R4 and R5 are selected from the group consisting of H and fluorine;
R7 is selected from the group consisting of H, C₁-C₄ alkyl, halogen, such as fluorine and chlorine, C₁-C₄ alkoxy, fluoroalkyl, fluoroalkoxy and C₁-C₄ alkylamino;
Y is selected from the group consisting of oxygen and sulfur;
HetAr is selected from the group consisting of 5-membered heteroaryl, 6-membered heteroaryl, and a bicyclic heteroaromatic ring system and HetAr may be substituted with one or more independently selected R7 substituents; and
wherein when R1 is C₁-C₄ alkoxy, in particular methoxy, it may form a ring closure with R2 or R6 when any one of these is C₁-C₄ alkyl, in particular methyl; or a pharmaceutically acceptable salt thereof.
2. The Compound (I) according to embodiment 1, or a pharmaceutically acceptable salt thereof, wherein R1 is selected from the group consisting of hydrogen, methyl, difluoromethyl, trifluoromethyl, fluorine, chlorine and methoxy.
3. The Compound (I) according to any of embodiments 1 and 2, or a pharmaceutically acceptable salt thereof, wherein R2 and R6 independently are selected from the group consisting of hydrogen, fluorine, bromine, chlorine, methoxy and methyl.
4. The Compound (I) according to any of embodiments 1 to 3, or a pharmaceutically acceptable salt thereof, wherein R3 is selected from the group consisting of hydrogen and methyl.
5. The Compound (I) according to any of embodiments 1 to 4, or a pharmaceutically acceptable salt thereof, wherein R4 and R5 independently are selected from the group consisting of hydrogen, methyl and fluorine.
6. The Compound (I) according to any of embodiments 1 to 5, or a pharmaceutically acceptable salt thereof, wherein R7 is selected from the group consisting of hydrogen, chlorine, fluorine, methyl, methoxy and methylamino.
7. The Compound (I) according to any of embodiments 1 to 6, or a pharmaceutically acceptable salt thereof, wherein HetAr is selected from the group consisting of pyrimidinyl, pyridazinyl, pyrazinyl, pyrazolyl, pyridyl, oxadiazolyl, isoxazolyl, oxazolyl, thiazolyl, imidazolyl, triazolyl, thiadiazolyl and imidazopyrimidinyl, in particular imidazo[1,2-a]pyrimidinyl.
8. The Compound (I) according to any of embodiments 1 to 7, or a pharmaceutically acceptable salt thereof, wherein Y is oxygen.
9. The Compound (I) according to any of embodiments 1 to 8, or a pharmaceutically acceptable salt thereof, selected from the group consisting of N-[(5-methylpyrimidin-2-yl)methyl]-1-(p-tolylsulfonyl)pyrrole-3-carboxamide, N-[(2-methylpyrimidin-5-yl)methyl]-1-(p-tolylsulfonyl)pyrrole-3-carboxamide, N-[(6-methylpyridazin-3-yl)methyl]-1-(p-tolylsulfonyl)pyrrole-3-carboxamide, 1-(2-fluorophenyl)sulfonyl-N-[(5-methylpyrazin-2-yl)methyl]pyrrole-3-carboxamide, 1-(3-fluorophenyl)sulfonyl-N-[(5-methylpyrazin-2-yl)methyl]pyrrole-3-carboxamide, 1-(4-fluorophenyl)sulfonyl-N-[(5-methylpyrazin-2-yl)methyl]pyrrole-3-carboxamide, 1-(4-methoxyphenyl)sulfonyl-N-[(5-methylpyrazin-2-yl)methyl]pyrrole-3-carboxamide, 4-methyl-N-[(5-methylpyrazin-2-yl)methyl]-1-(p-tolylsulfonyl)pyrrole-3-carboxamide, 1-(p-tolylsulfonyl)-N-(2-pyridylmethyl) pyrrole-3-carboxamide, N-[(3-methoxy-2-pyridyl)methyl]-1-(p-tolylsulfonyl)pyrrole-3-carboxamide, N-[(3-fluoro-2-pyridyl)methyl]-1-(p-tolylsulfonyl) pyrrole-3-carboxamide, N-[(4-fluoro-2-pyridyl)methyl]-1-(p-tolylsulfonyl) pyrrole-3-carboxamide, N-[(5-fluoro-2-pyridyl)methyl]-1-(p-tolylsulfonyl) pyrrole-3-carboxamide, 1-(p-tolylsulfonyl)-N-(3-pyridylmethyl) pyrrole-3-carboxamide, N-[(6-methyl-2-pyridyl)methyl]-1-(p-tolylsulfonyl)pyrrole-3-carboxamide, N-[(4-methyl-2-pyridyl)methyl]-1-(p-tolylsulfonyl)pyrrole-3-carboxamide, N-[(3-methyl-2-pyridyl)methyl]-1-(p-tolylsulfonyl)pyrrole-3-carboxamide, N-[(5-methoxy-2-pyridyl)methyl]-1-(p-tolylsulfonyl) pyrrole-3-carboxamide, N-[(4-methoxy-2-pyridyl)methyl]-1-(p-tolylsulfonyl) pyrrole-3-carboxamide, N-(imidazo[1,2-a]pyrimidin-6-ylmethyl)-1-(p-tolylsulfonyl)pyrrole-3-carboxamide, N-[(5-methylpyrazin-2-yl)methyl]-1-(p-tolylsulfonyl) pyrrole-3-carboxamide, N-[(6-methyl-3-pyridyl)methyl]-1-(p-tolylsulfonyl)pyrrole-3-carboxamide, N-[(5-methyl-2-pyridyl)methyl]-1-(p-tolylsulfonyl)pyrrole-3-carboxamide, N-[(5-methylpyrazin-2-yl)methyl]-1-(o-tolylsulfonyl) pyrrole-3-carboxamide, 1-(p-tolylsulfonyl)-N-(pyrazin-2-ylmethyl) pyrrole-3-carboxamide, N-[(5-methylpyrazin-2-yl)methyl]-1-(m-tolylsulfonyl)pyrrole-3-carboxamide, N-[(5-methyl-1,3,4-oxadiazol-2-yl)methyl]-1-(p-tolylsulfonyl)pyrrole-3-carboxamide, N-[(5-methylisoxazol-3-yl)methyl]-1-(p-tolylsulfonyl) pyrrole-3-carboxamide, N-[(5-methyloxazol-2-yl)methyl]-1-(p-tolylsulfonyl)pyrrole-3-carboxamide, N-[(4-methylthiazol-2-yl)methyl]-1-(p-tolylsulfonyl)pyrrole-3-carboxamide, N-[(3-methylisoxazol-5-yl)methyl]-1-(p-tolylsulfonyl) pyrrole-3-carboxamide, N-[(1-methylpyrazol-3-yl)methyl]-1-(p-tolylsulfonyl) pyrrole-3-carboxamide, N-[(1-methylpyrazol-4-yl)methyl]-1-(p-tolylsulfonyl) pyrrole-3-carboxamide, N-[(2-methyloxazol-5-yl)methyl]-1-(p-tolylsulfonyl)pyrrole-3-carboxamide, N-[(5-methylthiazol-2-yl)methyl]-1-(p-tolylsulfonyl)pyrrole-3-carboxamide, N-[(1-methylimidazol-4-yl)methyl]-1-(p-tolylsulfonyl)pyrrole-3-carboxamide, N-[(1-methyltriazol-4-yl)methyl]-1-(p-tolylsulfonyl)pyrrole-3-carboxamide, N-[(1-methyl-1,2,4-triazol-3-yl)methyl]-1-(p-tolylsulfonyl)pyrrole-3-carboxamide, N-[(3-methyl-1,2,4-oxadiazol-5-yl)methyl]-1-(p-tolylsulfonyl)pyrrole-3-carboxamide, 1-(4-methylbenzene-1-sulfonyl)-N-[(2-methyl-1,3-oxazol-4-yl)methyl]-1H-pyrrole-3-carboxamide, 1-(benzenesulfonyl)-N-[(5-methylpyrazin-2-yl)methyl]-1H-pyrrole-3-carboxamide, 1-(4-methylbenzene-1-sulfonyl)-N-[(1,3-thiazol-4-yl)methyl]-1H-pyrrole-3-carboxamide, 1-(4-methylbenzene-1-sulfonyl)-N-[(1,3-oxazol-5-yl)methyl]-1H-pyrrole-3-carboxamide, 1-(4-methylbenzene-1-sulfonyl)-N-[(1,3-thiazol-2-yl)methyl]-1H-pyrrole-3-carboxamide, 1-(4-methylbenzene-1-sulfonyl)-N-[(1,2-oxazol-3-yl)methyl]-1H-pyrrole-3-carboxamide, 1-(4-methylbenzene-1-sulfonyl)-N-[(1,2-oxazol-5-yl)methyl]-1H-pyrrole-3-carboxamide, 1-(4-methylbenzene-1-sulfonyl)-N-[(1,3-oxazol-4-yl)methyl]-1H-pyrrole-3-carboxamide, 1-(4-methylbenzene-1-sulfonyl)-N-[(1,2-thiazol-4-yl)methyl]-1H-pyrrole-3-carboxamide, 1-(4-methylbenzene-1-sulfonyl)-N-[(1,3,4-thiadiazol-2-yl)methyl]-1H-pyrrole-3-carboxamide, 1-(4-methylbenzene-1-sulfonyl)-N-[(1,2,4-oxadiazol-3-yl)methyl]-1H-pyrrole-3-carboxamide, 1-(4-methylbenzene-1-sulfonyl)-N-[(pyrimidin-5-yl)methyl]-1H-pyrrole-3-carboxamide, 1-(2-fluorobenzene-1-sulfonyl)-N-[(pyrazin-2-yl)methyl]-1H-pyrrole-3-carboxamide, 1-(3-methylbenzene-1-sulfonyl)-N-[(1-methyl-1H-pyrazol-3-yl)methyl]-1H-pyrrole-3-carboxamide, 1-(3-methylbenzene-1-sulfonyl)-N-[(3-methyl-1,2,4-oxadiazol-5-yl)methyl]-1H-pyrrole-3-carboxamide, 1-(3-methylbenzene-1-sulfonyl)-N-[(5-methylpyrimidin-2-yl)methyl]-1H-pyrrole-3-carboxamide, 1-(4-fluorobenzene-1-sulfonyl)-N-[(1-methyl-1H-pyrazol-3-yl)methyl]-1H-pyrrole-3-carboxamide, 1-(4-fluorobenzene-1-sulfonyl)-N-[(pyrazin-2-yl)methyl]-1H-pyrrole-3-carboxamide, 1-(4-fluorobenzene-1-sulfonyl)-N-[(3-methyl-1,2,4-oxadiazol-5-yl)methyl]-1H-pyrrole-3-carboxamide, 1-(4-methoxybenzene-1-sulfonyl)-N-[(1-methyl-1H-pyrazol-3-yl)methyl]-1H-pyrrole-3-carboxamide, 1-(4-methoxybenzene-1-sulfonyl)-N-[(pyrazin-2-yl)methyl]-1H-pyrrole-3-carboxamide, 1-(4-methoxybenzene-1-sulfonyl)-N-[(3-methyl-1,2,4-oxadiazol-5-yl)methyl]-1H-pyrrole-3-carboxamide 1-(4-methoxybenzene-1-sulfonyl)-N-[(1-methyl-1H-pyrazol-4-yl)methyl]-1H-pyrrole-3-carboxamide, 1-(4-methoxybenzene-1-sulfonyl)-N-[(5-methylpyridin-2-yl)methyl]-1H-pyrrole-3-carboxamide, 1-(4-methoxybenzene-1-sulfonyl)-N-[(3-methyl-1,2-oxazol-5-yl)methyl]-1H-pyrrole-3-carboxamide, 1-(4-methoxybenzene-1-sulfonyl)-N-[(5-methylpyrimidin-2-yl)methyl]-1H-pyrrole-3-carboxamide, 1-(4-methoxybenzene-1-sulfonyl)-N-[(5-methyl-1,3-oxazol-2-yl)methyl]-1H-pyrrole-3-carboxamide, 1-(2-methylbenzene-1-sulfonyl)-N-[(1-methyl-1H-pyrazol-3-yl)methyl]-1H-pyrrole-3-carboxamide, 1-(2-methylbenzene-1-sulfonyl)-N-[(3-methyl-1,2,4-oxadiazol-5-yl)methyl]-1H-pyrrole-3-carboxamide, 1-(2-methylbenzene-1-sulfonyl)-N-[(1-methyl-1H-pyrazol-4-yl)methyl]-1H-pyrrole-3-carboxamide, 1-(2-methylbenzene-1-sulfonyl)-N-[(5-methylpyridin-2-yl)methyl]-1H-pyrrole-3-carboxamide, 1-(2-methylbenzene-1-sulfonyl)-N-[(3-methyl-1,2-oxazol-5-yl)methyl]-1H-pyrrole-3-carboxamide, 1-(2-methylbenzene-1-sulfonyl)-N-[(5-methyl-1,3-oxazol-2-yl)methyl]-1H-pyrrole-3-carboxamide, 1-(4-chlorobenzene-1-sulfonyl)-N-[(pyrazin-2-yl)methyl]-1H-pyrrole-3-carboxamide, 1-(benzenesulfonyl)-N-[(1-methyl-1H-pyrazol-3-yl)methyl]-1H-pyrrole-3-carboxamide, 1-(benzenesulfonyl)-N-[(5-methylpyridin-2-yl)methyl]-1H-pyrrole-3-carboxamide, 1-(benzenesulfonyl)-N-[(3-methyl-1,2-oxazol-5-yl)methyl]-1H-pyrrole-3-carboxamide, 1-(4-fluorobenzene-1-sulfonyl)-N-[(6-methylpyridin-3-yl)methyl]-1H-pyrrole-3-carboxamide, 1-(4-methylbenzene-1-sulfonyl)-N-[(1,3-oxazol-2-yl)methyl]-1H-pyrrole-3-carboxamide, 5-fluoro-1-(4-methylbenzene-1-sulfonyl)-N-[(5-methylpyrazin-2-yl)methyl]-1H-pyrrole-3-carboxamide, 2-fluoro-1-(4-methylbenzene-1-sulfonyl)-N-[(5-methylpyrazin-2-yl)methyl]-1H-pyrrole-3-carboxamide, N-[(5-chloropyrazin-2-yl)methyl]-1-(4-methylbenzene-1-sulfonyl)-1H-pyrrole-3-carboxamide, 1-(4-fluoro-2-methylbenzene-1-sulfonyl)-N-[(5-methylpyrazin-2-yl)methyl]-1H-pyrrole-3-carboxamide, N-[(5-methylpyrazin-2-yl)methyl]-1-[4-(trifluoromethyl)benzene-1-sulfonyl]-1H-pyrrole-3-carboxamide, 1-(3-chloro-4-fluorobenzene-1-sulfonyl)-N-[(5-methylpyrazin-2-yl)methyl]-1H-pyrrole-3-carboxamide, 1-[4-(difluoromethyl)benzene-1-sulfonyl]-N-[(5-methylpyrazin-2-yl)methyl]-1H-pyrrole-3-carboxamide, 1-(4-methylbenzene-1-sulfonyl)-N-[(5-methylpyrazin-2-yl)methyl]-1H-pyrrole-3-carbothioamide, 1-(2-fluoro-4-methyl-phenyl)sulfonyl-N-[(5-methylpyrazin-2-yl)methyl]pyrrole-3-carboxamide, 1-(2-fluoro-4-methoxy-phenyl)sulfonyl-N-[(5-methylpyrazin-2-yl)methyl]pyrrole-3-carboxamide, 1-(3-fluoro-4-methoxy-phenyl)sulfonyl-N-[(5-methylpyrazin-2-yl)methyl]pyrrole-3-carboxamide, 1-(4-methoxy-2-methyl-phenyl)sulfonyl-N-[(5-methylpyrazin-2-yl)methyl]pyrrole-3-carboxamide, 1-(4-fluoro-2,6-dimethyl-phenyl)sulfonyl-N-[(5-methylpyrazin-2-yl)methyl]pyrrole-3-carboxamide, 1-(4-fluoro-3,5-dimethyl-phenyl)sulfonyl-N-[(5-methylpyrazin-2-yl)methyl]pyrrole-3-carboxamide, 1-(4-fluoro-3-methyl-phenyl)sulfonyl-N-[(5-methylpyrazin-2-yl)methyl]pyrrole-3-carboxamide, 1-(2,3-dihydrobenzofuran-5-ylsulfonyl)-N-[(5-methylpyrazin-2-yl)methyl]pyrrole-3-carboxamide, N-[(5-methylpyrazin-2-yl)methyl]-1-(2,4,6-trimethylphenyl)sulfonyl-pyrrole-3-carboxamide, 1-(2-chloro-4-methoxy-phenyl)sulfonyl-N-[(5-methylpyrazin-2-yl)methyl]pyrrole-3-carboxamide, 1-(2-bromo-4-methoxy-phenyl)sulfonyl-N-[(5-methylpyrazin-2-yl)methyl]pyrrole-3-carboxamide, 1-(2-fluoro-4-methylbenzene-1-sulfonyl)-N-{[5-(methylamino)pyrazin-2-yl]methyl}-1H-pyrrole-3-carboxamide, 1-[4-(difluoromethoxy)benzene-1-sulfonyl]-N-{[5-(methylamino)pyrazin-2-yl]methyl}-1H-pyrrole-3-carboxamide, 1-(2-fluoro-4-methylbenzene-1-sulfonyl)-N-[(2-methylpyrimidin-5-yl)methyl]-1H-pyrrole-3-carboxamide, 1-(4-methylbenzene-1-sulfonyl)-N-[(2-methyl-2H-1,2,3-triazol-4-yl)methyl]-1H-pyrrole-3-carboxamide, 1-(2-fluoro-4-methylbenzene-1-sulfonyl)-N-[(2-methoxypyrimidin-5-yl)methyl]-1H-pyrrole-3-carboxamide, 1-(benzenesulfonyl)-N-[(3,5-dimethylpyrazin-2-yl)methyl]-1H-pyrrole-3-carboxamide, 1-[4-(difluoromethoxy)benzene-1-sulfonyl]-N-[(2-methoxypyrimidin-5-yl)methyl]-1H-pyrrole-3-carboxamide, 1-(benzenesulfonyl)-N-[(3-chloro-5-methylpyrazin-2-yl)methyl]-1H-pyrrole-3-carboxamide, 1-(4-methylbenzene-1-sulfonyl)-N-[(2-methyl-1,3-thiazol-5-yl)methyl]-1H-pyrrole-3-carboxamide, 1-(4-methylbenzene-1-sulfonyl)-N-[(5-methyl-1,3,4-thiadiazol-2-yl)methyl]-1H-pyrrole-3-carboxamide, 1-(4-methylbenzene-1-sulfonyl)-N-[(3-methyl-1H-pyrazol-5-yl)methyl]-1H-pyrrole-3-carboxamide, 1-(2-chloro-4-methoxybenzene-1-sulfonyl)-N-[(1-methyl-1H-pyrazol-3-yl)methyl]-1H-pyrrole-3-carboxamide, and 1-(2-chloro-4-methoxybenzene-1-sulfonyl)-N-[(5-methylpyrimidin-2-yl)methyl]-1H-pyrrole-3-carboxamide,
10. A pharmaceutical composition comprising Compound (I) of any of embodiments 1-9, or a pharmaceutically acceptable salt thereof, and one or more pharmaceutically acceptable excipients.
11. The Compound (I) of any of embodiments 1-9, or a pharmaceutically acceptable salt thereof, or the pharmaceutical composition of embodiment 10, for use in therapy.
12. The Compound (I) of any of embodiments 1-9, or a pharmaceutically acceptable salt thereof, or the pharmaceutical composition of embodiment 10, for use in a method for the treatment of a neurological or psychiatric disorder.
13. A method for the treatment of a neurological or psychiatric disorder comprising the administration of a therapeutically effective amount of Compound (I) of any of embodiments 1-9, or a pharmaceutically acceptable salt thereof, or the pharmaceutical composition of embodiment 10, to a patient in need thereof.
14. Use of Compound (I) of any of embodiments 1-9, or a pharmaceutically acceptable salt thereof, or the pharmaceutical composition of embodiment 10, for the manufacture of a medicament for the treatment of a neurological or psychiatric disorder.
15. The Compound (I) of any of embodiments 1-9, or a pharmaceutically acceptable salt thereof, for the use specified in embodiment 12, wherein the neurological or psychiatric disorder is selected from the group consisting of epilepsy, schizophrenia, for example of the paranoid, disorganized, catatonic, undifferentiated, or residual type; schizophreniform disorder; schizoaffective disorder, for example of the delusional type or the depressive type, cognitive impairment associated with schizophrenia (CIAS), autism spectrum disorder, bipolar disorder, ADHD, anxiety-related disorders, depression, cognitive dysfunction, Alzheimer's disease, fragile X syndrome, chronic pain, hearing loss, sleep and circadian disorders, sleep disruption and movement disorders, such as Huntington's disease, L-dopa-induced dyskinesia, obsessive compulsive disorders, and Tourette syndrome.
16. The pharmaceutical composition of embodiment 10 for the use specified in embodiment 12, wherein the neurological or psychiatric disorder is selected from the group consisting of epilepsy, schizophrenia, for example of the paranoid, disorganized, catatonic, undifferentiated, or residual type; schizophreniform disorder; schizoaffective disorder, for example of the delusional type or the depressive type, cognitive impairment associated with schizophrenia (CIAS), autism spectrum disorder, bipolar disorder, ADHD, anxiety-related disorders, depression, cognitive dysfunction, Alzheimer's disease, fragile X syndrome, chronic pain, hearing loss, sleep and circadian disorders, sleep disruption and movement disorders, such as Huntington's disease, L-dopa-induced dyskinesia, obsessive compulsive disorders, and Tourette syndrome.
17. Use of Compound (I) of any of embodiments 1-9, or a pharmaceutically acceptable salt thereof, for the manufacture of a medicament for the treatment of a neurological or psychiatric disorder, wherein the neurological or psychiatric disorder is selected from the group consisting of epilepsy, schizophrenia, for example of the paranoid, disorganized, catatonic, undifferentiated, or residual type; schizophreniform disorder; schizoaffective disorder, for example of the delusional type or the depressive type, cognitive impairment associated with schizophrenia (CIAS), autism spectrum disorder, bipolar disorder, ADHD, anxiety-related disorders, depression, cognitive dysfunction, Alzheimer's disease, fragile X syndrome, chronic pain, hearing loss, sleep and circadian disorders, sleep disruption and movement disorders, such as Huntington's disease, L-dopa-induced dyskinesia, obsessive compulsive disorders, and Tourette syndrome.
18. The Compound (I) of any of embodiments 1-9 provided that the compound is not N-[(5-methylpyrazin-2-yl)methyl]-1-(p-tolylsulfonyl)pyrrole-3-carboxamide.
19. The Compound (I) of either of embodiments 10-11 provided that the compound is not N-[(5-methylpyrazin-2-yl)methyl]-1-(p-tolylsulfonyl)pyrrole-3-carboxamide.

### EXPERIMENTAL SECTION

The compounds of formula I may be prepared by methods described below, together with synthetic methods known in the art of organic chemistry, or modifications that are familiar to those skilled in the art. The starting materials used herein are available commercially or may be prepared by routine methods known in the art, such as those methods described in standard reference books such as “Compendium of Organic Synthetic Methods, Vol. I-XII” (published by Wiley-Interscience). Preferred methods include, but are not limited to, those described below.

The schemes are representative of methods useful in synthesizing the compounds of the present invention. They are not to constrain the scope of the invention in any way.

### Analytical Methods

Chromatographic systems and methods to evaluate chemical purity (LCMS methods) are described below:

Method A: Apparatus: Agilent 1200 LCMS
system with ELS Detector.

| Column | Waters Xbridge-C18, 50 × 2 mm, 5 μm | |
|---|---|---|
| Flow rate | 0.8 | mL/min |
| Run time | 4.5 | min. |
| Wavelenght | 254 | nm |
| Oven temp | 50° | C. |

| Ion source | ESI | | |
|---|---|---|---|
| Solvent A | Water + 0.04% TFA | | |
| Solvent B | CH₃CN (MeCN) + 0.02% TFA | | |
| Gradient | Time | A % | B % |
| | 0 | 99 | 1 |
| | 3.4 | 0 | 100 |
| | 4 | 99 | 1 |
| | 4.5 | 99 | 1 |

Method B: Apparatus: Agilent 1200 LCMS
system with ELS Detector..

| Column | Waters XBridge ShieldRP18, 2.1*50 mm, 5 μm | |
|---|---|---|
| Flow rate | 0.8 | mL/min |
| Run time | 4.5 | min. |
| Wavelenght | 254 | nm |
| Oven temp | 40° | C. |

| Ion source | ESI | | |
|---|---|---|---|
| Solvent A | Water + 0.05% NH₃•H₂O | | |
| Solvent B | CH₃CN (MeCN) | | |
| Gradient | Time | A % | B % |
| | 0 | 95 | 5 |
| | 3.4 | 0 | 100 |
| | 4 | 0 | 100 |
| | 4.5 | 95 | 5 |

Method C: Waters Aquity UPLC with TQD MS-detector

| Column | Aquity UPLC BEH C18, 2.1*50 mm, 1.7 μm | | |
|---|---|---|---|
| Flow rate | 1.2 | mL/min | |
| Run time | 1.15 | min. | |
| Wavelenght | 254 | nm | |
| Oven temp | 60° | C. | |
| Ion source | ESI | | |
| Solvent A | Water + 0.05% TFA | | |
| Solvent B | 0.035% TFA in CH₃CN 95% + Water 5% | | |
| Gradient | Time | A % | B % |
| | 0 | 90 | 10 |
| | 1 | 0 | 100 |
| | 1.15 | 90 | 10 |

Method D: Waters Aquity UPLC with TQD MS-detector

| Column | Aquity UPLC BEH C18, 2.1*50 mm, 1.7 μm | | |
|---|---|---|---|
| Flow rate | 1.2 | mL/min | |
| Run time | 1.15 | min. | |
| Wavelenght | 254 | nm | |
| Oven temp | 60° | C. | |
| Ion source | APPI | | |
| Solvent A | Water + 0.05% TFA | | |
| Solvent B | 0.035% TFA in CH₃CN 95% + Water 5% | | |
| Gradient | Time | A % | B % |
| | 0 | 90 | 10 |
| | 1 | 0 | 100 |
| | 1.15 | 90 | 10 |

Following separation by chromatography the compounds were analysed by use of 1H NMR. 1H NMR spectra were recorded at 400.13 MHz on a Bruker Avance III 400 instrument, at 300.13 MHz on a Bruker Avance 300 instrument or at 600.16 MHz on a 600 MHz Bruker Avance III HD. Deuterated dimethyl sulfoxide or deuterated chloroform was used as solvent. Tetramethylsilane was used as internal reference standard.

Chemical shift values are expressed in ppm-values relative to tetramethylsilane. The following abbreviations are used for multiplicity of NMR signals: s=singlet, d=doublet, t=triplet, q=quartet, qui=quintet, h=heptet, dd=double doublet, dt=double triplet, dq=double quartet, tt=triplet of triplets, m=multiplet and brs=broad singlet.

### Synthesis of Compounds of the Invention

### General Methods:

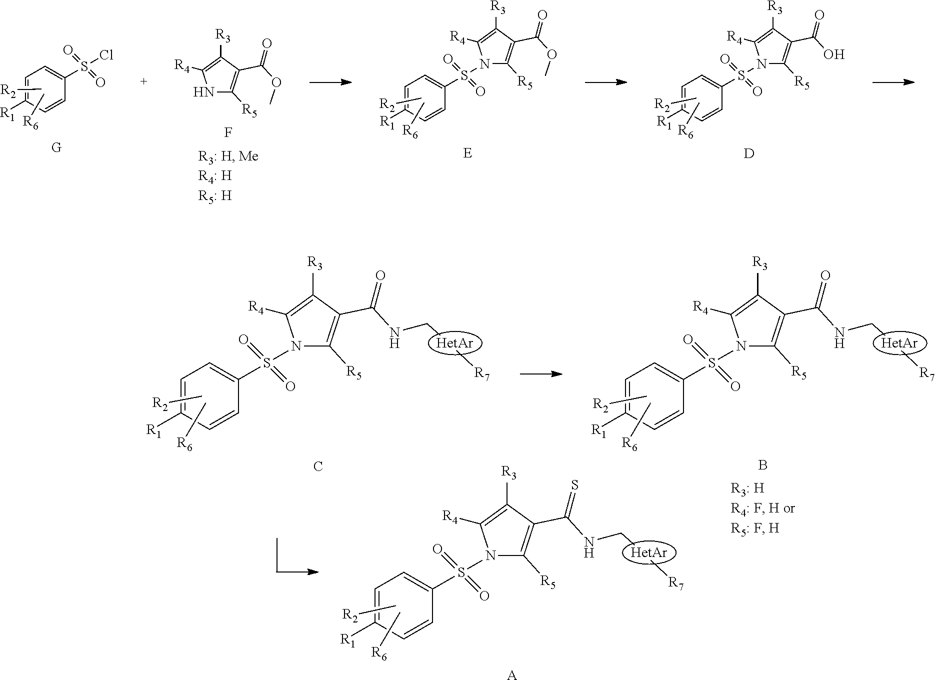

In brief, compounds of the invention can be prepared starting from a commercially available pyrrolo carboxylic acid ester (F), such as 1H-methyl-1H-pyrrole-3-carboxylic acid methyl ester (CAS 40318-15-8) or 1H-Pyrrole-3-carboxylic acid methyl ester (CAS 2703-17-5). Compound of the formula E can be prepared by reacting F with an arylsulfonic acid derivative exemplified by, but not limited to, an arylsulfonylchloride (G) in a solvent such as tetrahydrofuran, in the presence of a base exemplified by, but not limited to, sodium hydride. Intermediate D can be prepared from E under standard hydrolysis conditions, exemplified by but not limited to aqueous lithium hydroxide in tetrahydrofuran. Compound C is formed from intermediate D by coupling with an amine under standard amide formation conditions, using a coupling reagent, such as HATU (hexafluorophosphate azabenzotriazole tetramethyl uronium), and a base exemplified by, but not limited to, triethylamine, in a solvent exemplified by, but not limited to, dichloromethane. Compounds of the formula B can be prepared from C using an electrophilic fluorination agent exemplified by, but not limited to, N-fluoro-N-(chloromethyl)triethylenediamine bis(tetrafluoroborate) in a solvent such as acetonitrile. Compounds of the formula A can be prepared from C by treatment with 2,4-bis-(4-methoxy-phenyl)-[1,3,2,4]dithiadiphosphetane 2,4-disulfide in a solvent exemplified by, but not limited to, toluene.

### Example 1

### Synthesis of Compound 8

### Preparation of methyl-4-methyl-1-(p-tolylsulfonyl)pyrrole-3-carboxylate

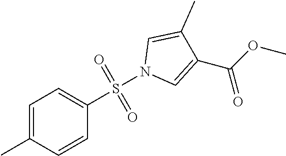

To a solution of methyl-4-methyl-1H-pyrrole-3-carboxylate (300 mg, 2.2 mmol) in THF (5 mL) was added NaH (104 mg, 2.6 mmol, 60% in mineral oil) at −40° C. under N₂. The mixture was stirred at 20° C. for 1 hour, then 4-methylbenzenesulfonyl chloride (411 mg, 2.2 mmol) was added at 0° C. and the reaction mixture was allowed to warm to 20° C. and stirred for 2 hours. The reaction was quenched with saturated NH₄Cl solution (aq, 10 ml). The aqueous phase was extracted with ethyl acetate (30 mL×2). The combined organic phases were washed with brine (30 mL×2), dried with anhydrous Na₂SO₄, filtered and concentrated in vacuo. The residue was purified by silica gel column chromatography (petroleum ether/ethyl acetate) to afford methyl-4-methyl-1-(p-tolylsulfonyl)pyrrole-3-carboxylate (464 mg, 73% yield).

### Preparation of 4-methyl-1-(p-tolylsulfonyl)pyrrole-3-carboxylic acid

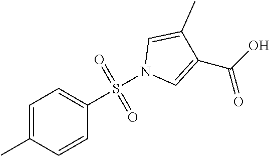 [SMILES: Cc1ccc(S(=O)(=O)n2cc(C)c(C(=O)O)c2)cc1]

To a solution of methyl-4-methyl-1-(p-tolylsulfonyl) pyrrole-3-carboxylate (200 mg, 0.68 mmol) in THF (4 mL) and H₂O (2 mL) was added LiOH—H₂O (588 mg, 1.36 mmol) at 20° C. under N₂. The mixture was stirred at 20° C. for 12 hours. The reaction was acidified to pH=5 using HCl (aq, 2 mol/L), and extracted with ethyl acetate (20 mL×2). The combined organic phases were washed with brine (30 mL×2), dried with anhydrous Na₂SO₄, filtered and concentrated to afford 4-methyl-1-(p-tolylsulfonyl)pyrrole-3-carboxylic acid (210 mg, crude) which was used in the next step directly.

### 4-Methyl-N-[(5-methylpyrazin-2-yl)methyl]-1-(p-tolylsulfonyl)pyrrole-3-carboxamide (compound 8)

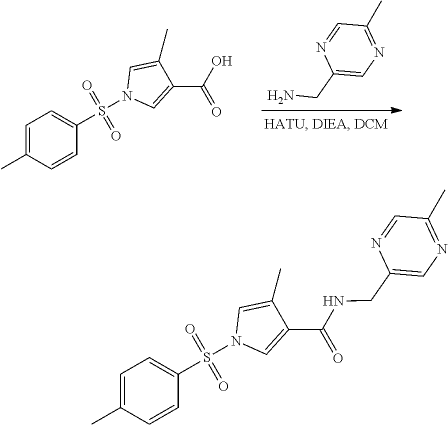

To a mixture of (5-methylpyrazin-2-yl)methanamine (168 mg, 1.36 mmol) and 4-methyl-1-(p-tolylsulfonyl)pyrrole-3-carboxylic acid (383 mg, 1.36 mmol) in DCM (10 mL) was added HATU (517 mg, 1.63 mmol) and DIEA (527 mg, 4.08 mmol) at 20° C. under N₂. The mixture was stirred at 20° C. for 12 hours and then concentrated to afford the crude product. The crude product was purified by preparative HPLC to afford 4-methyl-N-[(5-methylpyrazin-2-yl)methyl]-1-(p-tolylsulfonyl)pyrrole-3-carboxamide (65 mg, 24% yield).

¹H NMR (DMSO-d⁶ 400 MHz): δ 8.68 (t, 1H), 8.47 (s, 2H), 7.91 (d, 1H), 7.85 (d, 2H), 7.47 (d, 2H), 7.15 (s, 1H), 4.44 (d, 2H), 2.47 (s, 3H), 2.39 (s, 3H), 2.09 (s, 3H). LC-MS: t_{R}=2.286 min (method A), m/z=385.1 [M+H]⁺.

Compound 1 to 86 and 89-118 in table 1 were prepared by a similar method. For compound 111, (3,5-dimethylpyrazin-2-yl)methanamine was prepared from commercially available 2-chloro-3,5-dimethyl-pyrazine via palladium-catalyzed introduction of cyanid followed by reduction to the amine using Raney Ni.

### Example 2

### Preparation of 5-fluoro-1-(4-methylbenzene-1-sulfonyl)-N-[(5-methylpyrazin-2-yl)methyl]-1H-pyrrole-3-carboxamide (compound 87), and 2-fluoro-1-(4-methylbenzene-1-sulfonyl)-N-[(5-methylpyrazin-2-yl)methyl]-1H-pyrrole-3-carboxamide (compound 88)

N-Fluoro-N-(chloromethyl)triethylenediamine bis(tetrafluoroborate) (247 mg, 0.668 mmol) was added to N-((5-methylpyrazin-2-yl)methyl)-1-tosyl-1H-pyrrole-3-carboxamide (200 mg, 0.535 mmol) in acetonitrile (10 mL). The mixture was stirred at 70° C. under argon for 44 hours. The reaction mixture was diluted with water and extracted with ethyl acetate. The combined organic phases were washed with brine, dried over MgSO₄ and concentrated in vacuo. The crude material was purified by flash chromatography (ethyl acetate (containing 5% Et₃N)/heptane). A mixture of compound 87 and 88 was obtained. Further purification was performed using mass directed HPLC (see method below) and yielded:

First eluting peak 10 mg of compound 88 (5%):

LC-MS: t_{R}=0.63 min (method C), m/z=389.2 [M+H]⁺.

¹H NMR (600 MHz, Chloroform-d) δ 8.48 (d, 1H), 8.38 (d, 1H), 7.85 (d, 2H), 7.36 (d, 2H), 6.81 (d, 1H), 6.79 (dd, 1H), 6.49 (t, 1H), 4.65 (d, 2H), 2.55 (s, 3H), 2.45 (s, 3H).

Second eluting peak 10 mg compound 87 (5%):

LC-MS: t_{R}=0.64 min (method C), m/z=389.2 [M+H]⁺.

¹H NMR (600 MHz, Chloroform-d) δ 8.50 (d, 1H), 8.39 (d, 1H), 7.85 (d, 2H), 7.38-7.34 (m, 3H), 6.81 (t, 1H), 5.88 (dd, 1H), 4.66 (d, 2H), 2.56 (s, 3H), 2.44 (s, 3H).

Preparative LC-MS

Mass directed preparative LC-MS was performed on a Waters AutoPurification system equipped with a diode array detector and QDa mass detector operating in positive/negative mode. The column was Waters XSelect CSH Prep C18, 5 μm OBD, 30×100 mm.

Mobile phase A: Water+0.1% formic acid

Mobile phase B: Acetonitrile+0.1% formic acid

Flow: 70 ml/min, room temperature, total run length 5.0 min

Gradient:

T=0.0 min: 65% A

T=0.2 min: 65% A

T=4.0 min 55% A

T=4.1 min 10% A

T=4.5 min 65% A

### Example 3

### The preparation of 1-(4-methylbenzene-1-sulfonyl)-N-[(5-methylpyrazin-2-yl)methyl]-1H-pyrrole-3-carbothioamide (compound 94)

2,4-Bis-(4-methoxy-phenyl)-[1,3,2,4]dithiadiphosphetane 2,4-disulfide (134 mg, 0.324 mmol) was added to N-((5-methylpyrazin-2-yl)methyl)-1-tosyl-1H-pyrrole-3-carboxamide (100 mg, 0.270 mmol) in toluene (2.5 mL) under argon. The reaction mixture was heated at 160° C. for 30 minutes by microwave irradiation.

To the mixture was added water and the mixture was extracted with ethyl acetate. The organic phase was washed with brine, dried over MgSO₄ and concentrated in vacuo. The crude material was purified by flash chromatography (ethyl acetate (containing 5% Et₃N)/heptane) to afford 30 mg (26%) of 1-(4-methylbenzene-1-sulfonyl)-N-[(5-methylpyrazin-2-yl)methyl]-1H-pyrrole-3-carbothioamide (compound 94).

¹H NMR (600 MHz, Chloroform-d) δ 8.70 (t, 1H), 8.55 (d, 1H), 8.40 (d, 1H), 7.83-7.77 (m, 3H), 7.35-7.30 (m, 2H), 7.14 (dd, 1H), 6.68 (dd, 1H), 5.03 (d, 2H), 2.58 (s, 3H), 2.41 (s, 3H).

LC-MS: t_{R}=0.71 min (method D), m/z=387.1 [M+H]⁺.

Compounds of the Invention

TABLE 1

| | | | | LCMS | Reten- | |
|---|---|---|---|---|---|---|
| | | | | Ob- | tion | |
| | | | | served | time | |
| | Chemical Name | Structure | LCMS | Mass | (min) | NMR |
| 1 | N-[(5- methylpyrimidin- 2-yl)methyl]-1- (p-tolylsulfonyl) pyrrole-3- carboxamide | 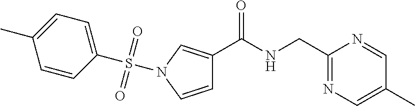 | A | 371.1 | 2.14 | ¹H NMR (CDCl₃ 400 MHz): δ 8.55 (s, 2H), 7.78 (d, 2H), 7.72 (t, 1H), 7.31 (d, 2H), 7.15 (t, 2H), 6.64-6.62 (m, 1H), 4.77 (d, |
| | | | | | | 2H), 2.41 (s, 3H), |
| | | | | | | 2.32 (s, 3H). |
| 2 | N-[(2- methylpyrimidin- 5-yl)methyl]-1- (p-tolylsulfonyl) pyrrole-3- carboxamide | 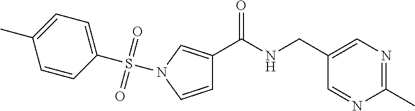 | B | 371.1 | 2.00 | ¹H NMR (DMSO-d⁶ 400 MHz): δ 8.69 (t, 1H), 8.57 (s, 2H), 7.87-7.83 (m, 3H), 7.43 (d, 2H), 7.36-7.34 (m, 1H), 6.65- |
| | | | | | | 6.64 (m, 1H), |
| | | | | | | 4.31 (d, 2H), 2.54 |
| | | | | | | (s, 3H), 2.35 (s, |
| | | | | | | 3H). |
| 3 | N-[(6- methylpyridazin- 3-yl)methyl]-1- (p-tolylsulfonyl) pyrrole-3- carboxamide | 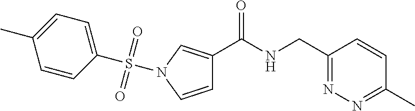 | A | 371.1 | 1.94 | ¹H NMR (DMSO-d⁶ 400 MHz): δ 8.84 (t, 1H), 7.87-7.85 (m, 3H), 7.47-7.41 (m, 4H), 7.37-7.35 (m, 1H), 6.69-6.68 (m, 1H), 4.58 (d, |
| | | | | | | 2H), 2.54 (s, 3H), |
| | | | | | | 2.35 (s, 3H). |
| 4 | 1-(2- fluorophenyl) sulfonyl-N-[(5- methylpyrazin- 2-yl)methyl] pyrrole-3- carboxamide | 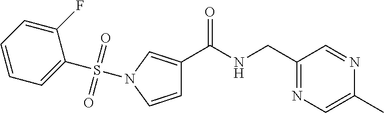 [SMILES: Cc1cnc(CNC(=O)c2ccn(S(=O)(=O)c3ccccc3F)c2)cn1] | A | 375.0 | 2.19 | ¹H NMR (CDCl₃ 400 MHz): δ 8.51 (s, 1H), 8.39 (s, 1H), 7.98 (t, 1H), 7.72 (s, 1H), 7.68-7.63 (m, 1H), 7.33 (t, 1H), 7.23-7.17 (m, 2H), |
| | | | | | | 6.87 (brs, 1H), |
| | | | | | | 6.61-6.60 (m, 1H), |
| | | | | | | 4.68 (d, 2H), 2.56 |
| | | | | | | (s, 3H). |
| 5 | 1-(3- fluorophenyl) sulfonyl-N-[(5- methylpyrazin- 2-yl)methyl] pyrrole-3- carboxamide | 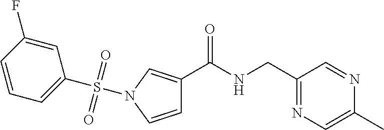 [SMILES: Cc1cnc(CNC(=O)c2ccn(S(=O)(=O)c3cccc(F)c3)c2)cn1] | A | 375.1 | 2.04 | ¹H NMR (CDCl₃ 400 MHz): δ 8.52 (s, 1H), 8.40 (s, 1H), 7.72-7.70 (m, 2H), 7.62-7.59 (m, 1H), 7.57-7.52 (m, 1H), 7.38-7.33 (m, 1H), 7.18-7.17 (m, 1H), 6.86 (brs, |
| | | | | | | 1H), 6.62-6.61 (m, |
| | | | | | | 1H), 4.67 (d, 2H), |
| | | | | | | 2.58 (s, 3H). |
| 6 | 1-(4- fluorophenyl) sulfonyl-N-[(5- methylpyrazin- 2-yl)methyl] pyrrole-3- carboxamide | 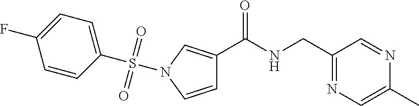 [SMILES: Cc1cnc(CNC(=O)c2ccn(S(=O)(=O)c3ccc(F)cc3)c2)cn1] | A | 375.1 | 2.03 | ¹H NMR (DMSO-d⁶ 400 MHz): δ 8.82 (t, 1H), 8.46 (s, 2H), 8.12 (dd, 2H), 7.93 (s, 1H), 7.53 (t, 2H), 7.43 (t, 1H), 6.74 (t, |
| | | | | | | 1H), 4.48 (d, 2H), |
| | | | | | | 2.46 (s, 3H). |
| 7 | 1-(4- methoxyphenyl) sulfonyl-N-[(5- methylpyrazin- 2-yl)methyl] pyrrole-3- carboxamide | 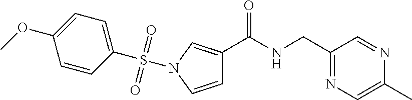 | A | 387.1 | 2.06 | ¹H NMR (CDCl₃ 400 MHz): δ 8.51 (s, 1H), 8.38 (s, 1H), 7.83 (d, 2H), 7.68 (t, 1H), 7.14 (t, 1H), 6.96 (d, 2H), 6.83 (m, 1H), 6.55-6.56 (m, |
| | | | | | | 1H), 4.67 (d, 2H), |
| | | | | | | 3.86 (s, 3H), |
| | | | | | | 2.56 (s, 3H). |
| 8 | 4-methyl-N-[(5- methylpyrazin- 2-yl)methyl]-1- (p-tolylsulfonyl) pyrrole-3- carboxamide | 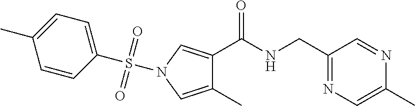 | A | 385.1 | 2.29 | ¹H NMR (DMSO-d⁶ 400 MHz): δ 8.68 (t, 1H), 8.47 (s, 2H), 7.91 (d, 1H), 7.85 (d, 2H), 7.47 (d, 2H), 7.15 (s, 1H), 4.44 (d, 2H), |
| | | | | | | 2.47 (s, 3H), 2.39 |
| | | | | | | (s, 3H), 2.09 (s, |
| | | | | | | 3H). |
| 9 | 1-(p- tolylsulfonyl)- N-(2- pyridylmethyl) pyrrole-3- carboxamide | 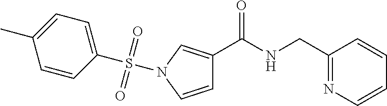 [SMILES: Cc1ccc(S(=O)(=O)n2ccc(C(=O)NCc3ccccn3)c2)cc1] | A | 356.1 | 1.84 | ¹H NMR (CDCl₃ 400 MHz): δ 8.53 (d, 1H), 7.76 (d, 2H), 7.70-7.65 (m, 2H), 7.30- 7.26 (m, 3H), 7.19-7.13 (m, 2H), 7.13-7.12 |
| | | | | | | (m, 1H), 6.59 |
| | | | | | | (dd, 1H), 4.65 |
| | | | | | | (d, 2H), 2.39 |
| | | | | | | (s, 3H). |
| 10 | N-[(3-methoxy- 2-pyridyl) methyl]-1-(p- tolylsulfonyl) pyrrole-3- carboxamide | 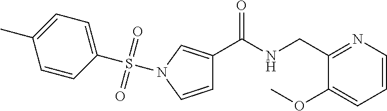 [SMILES: COc1cccnc1CNC(=O)c1ccn(S(=O)(=O)c2ccc(C)cc2)c1] | A | 385.9 | 1.78 | ¹H NMR (DMSO-d⁶ 400 MHz): δ 8.44 (t, 1H), 8.04 (dd, 1H), 7.90-7.87 (m, 3H), 7.45 (d, 2H), 7.38-7.35 (m, 2H), 7.25 |
| | | | | | | (dd, 1H), 6.71 |
| | | | | | | (dd, 1H), 4.46 |
| | | | | | | (d, 2H), 3.81 (s, |
| | | | | | | 3H), 2.36 (s, 3H). |
| 11 | N-[(3-fluoro- 2-pyridyl) methyl]-1-(p- tolylsulfonyl) pyrrole-3- carboxamide | 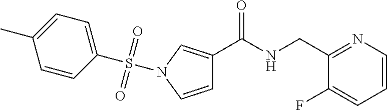 [SMILES: Cc1ccc(S(=O)(=O)n2ccc(C(=O)NCc3ncccc3F)c2)cc1] | A | 374.1 | 2.25 | ¹H NMR (CDCl₃ 400 MHz): δ 8.38 (d, 1H), 7.79 (d, 2H), 7.72 (t, 1H), 7.44-7.39 (m, 1H), 7.27-7.23 (m, 4H), 7.16 (dd, 1H), 6.63 (dd, 1H), |
| | | | | | | 4.75 (dd, 2H), |
| | | | | | | 2.41 (s, 3H). |
| 12 | N-[(4-fluoro- 2-pyridyl) methyl]-1-(p- tolylsulfonyl) pyrrole-3- carboxamide | 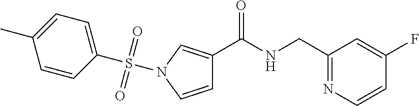 [SMILES: Cc1ccc(S(=O)(=O)n2ccc(C(=O)NCc3cc(F)ccn3)c2)cc1] | A | 374.1 | 2.08 | ¹H NMR (CDCl₃ 400 MHz): δ 8.52 (dd, 1H), 7.78 (d, 2H), 7.71 (t, 1H), 7.32 (d, 2H), 7.16 (dd, 1H), 7.04 (m, 2H), 6.98-6.94 (m, 1H), 6.59 (dd, |
| | | | | | | 1H), 4.67 (d, 2H), |
| | | | | | | 2.42 (s, 3H). |
| 13 | N-[(5-fluoro- 2-pyridyl) methyl]-1-(p- tolylsulfonyl) pyrrole-3- carboxamide | 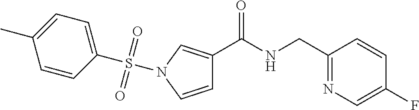 [SMILES: Cc1ccc(S(=O)(=O)n2ccc(C(=O)NCc3ccc(F)cn3)c2)cc1] | A | 374.1 | 2.29 | ¹H NMR (DMSO-d⁶ 400 MHz): δ 8.80 (t, 1H), 8.46 (d, 1H), 7.90-7.88 (m, 3H), 7.64 (td, 1H), 7.46 (d, 2H), 7.38 (dd, 1H), |
| | | | | | | 7.34 (dd, 1H), |
| | | | | | | 6.71 (dd, 1H), |
| | | | | | | 4.44 (d, 2H), |
| | | | | | | 2.37 (s, 3H). |
| 14 | 1-(p- tolylsulfonyl)- N-(3- pyridylmethyl) pyrrole-3- carboxamide | 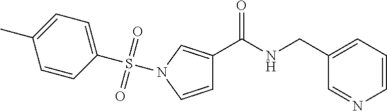 [SMILES: Cc1ccc(S(=O)(=O)n2ccc(C(=O)NCc3cccnc3)c2)cc1] | A | 356.1 | 1.80 | ¹H NMR (DMSO-d⁶ 400 MHz): δ 8.74 (t, 1H), 8.51 (s, 1H), 8.45 (dd, 1H), 7.90-7.88 (m, 3H), 7.69- 7.66 (m, 1H), 7.47 |
| | | | | | | (d, 2H), 7.39 (d, |
| | | | | | | 1H), 7.39-7.32 |
| | | | | | | (m, 1H), 6.71 |
| | | | | | | (dd, 1H), 4.40 |
| | | | | | | (d 2H), 2.39 (s, |
| | | | | | | 3H). |
| 15 | N-[(6-methyl- 2-pyridyl) methyl]-1-(p- tolylsulfonyl) pyrrole-3- carboxamide | 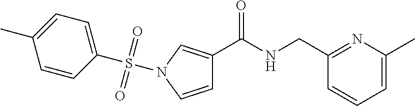 | A | 370.1 | 1.85 | ¹H NMR (CDCl₃ 400 MHz): δ 7.75 (d, 2H), 7.68 (s, 1H), 7.52 (t, 1H), 7.28 (d, 2H), 7.21 (brs, 1H), 7.13 (t, 1H), 7.08-7.03 (m, 2H), 6.57 |
| | | | | | | (dd, 1H), 4.60 (d, |
| | | | | | | 2H), 2.53 (s, 3H), |
| | | | | | | 2.39 (s, 3H). |
| 16 | N-[(4-methyl- 2-pyridyl) methyl]-1-(p- tolylsulfonyl) pyrrole-3- carboxamide | 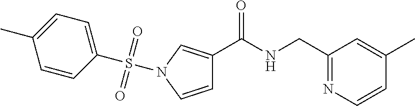 | B | 370.1 | 2.24 | ¹H NMR (DMSO-d⁶ 400 MHz): δ 8.75 (m, 1H), 8.30 (d, 1H), 7.89-7.87 (m, 3H), 7.45 (d, 2H), 7.36 (d, 1H), 7.07-7.04 |
| | | | | | | (m, 2H), 6.72 (s, |
| | | | | | | 1H), 4.39 (d, |
| | | | | | | 2H), 2.36 (s, |
| | | | | | | 3H), 2.25 (s, |
| | | | | | | 3H). |
| 17 | N-[(3-methyl- 2-pyridyl) methyl]-1-(p- tolylsulfonyl) pyrrole-3- carboxamide | 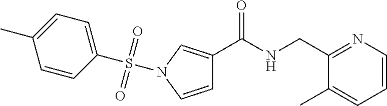 [SMILES: Cc1ccc(S(=O)(=O)n2ccc(C(=O)NCc3ncccc3C)c2)cc1] | A | 370.1 | 1.85 | ¹H NMR (DMSO-d⁶ 400 MHz): δ 8.55 (t, 1H), 8.30 (d, 1H), 7.93 (t, 1H), 7.90 (d, 2H), 7.56 (d, 1H), 7.48 (d, 2H), 7.39-7.37 |
| | | | | | | (m, 1H), 7.20 |
| | | | | | | (dd, 1H), 6.74- |
| | | | | | | 6.73 (m, 1H), 4.49 |
| | | | | | | (d, 2H), 2.39 (s, |
| | | | | | | 3H), 2.31 (s, 3H). |
| 18 | N-[(5-methoxy- 2-pyridyl) methyl]-1-(p- tolylsulfonyl) pyrrole-3- carboxamide | 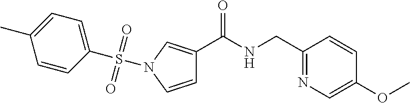 | A | 386.1 | 1.96 | ¹H NMR (DMSO-d⁶ 400 MHz): δ 8.71 (t, 1H), 8.16 (d, 1H), 7.87-7.85 (m, 3H), 7.44 (d, 2H), 7.35 (d, 1H), 7.29-7.28 |
| | | | | | | (m, 1H), 7.19 (d, |
| | | | | | | 1H), 6.69 (dd, |
| | | | | | | 1 H), 4.37 (d, |
| | | | | | | 2H), 3.76 (s, 3H), |
| | | | | | | 2.35 (s, 3H). |
| 19 | N-[(4-methoxy- 2-pyridyl) methyl]-1-(p- tolylsulfonyl) pyrrole-3- carboxamide | 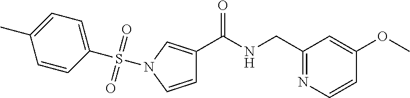 | A | 386.1 | 1.80 | ¹H NMR (CDCl₃ 400 MHz): δ 8.33 (d, 1H), 7.75 (d, 2H), 6.68 (t, 1H), 7.28 (d, 2H), 7.20 (s, 1H), 7.13-7.12 (m, 1H), 6.78 (d, |
| | | | | | | 1H), 6.72 (dd, |
| | | | | | | 1H), 6.58 (dd, |
| | | | | | | 1H), 4.59 (d, 2H), |
| | | | | | | 3.83 (s, 3H), 2.39 |
| | | | | | | (s, 3H). |
| 24 | N-(imidazo [1,2-a] pyrimidin-6- ylmethyl)- 1-(p- tolylsulfonyl) pyrrole-3- carboxamide | 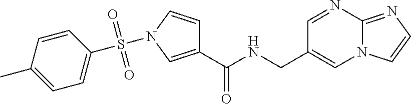 [SMILES: Cc1ccc(S(=O)(=O)n2ccc(C(=O)NCc3cnc4nccn4c3)c2)cc1] | D | 396 | 0.45 | nd |
| 25 | N-[(5-methyl- pyrazin-2-yl) methyl]-1-(p- tolylsulfonyl) pyrrole-3- carboxamide | 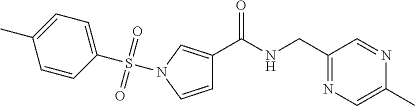 | A | 371.0 | 2.32 | ¹H NMR (CDCl₃ 400 MHz): δ 8.51 (s, 1H), 8.39 (s, 1H), 7.78 (d, 2H), 7.69 (t, 1H), 7.31 (d, 2H), 7.15 (t, 1H), 6.82 (brs, 1H), 6.56 (dd, |
| | | | | | | 1H), 4.67 (d, 2H), |
| | | | | | | 2.57 (s, 3H), 2.42 |
| | | | | | | (s, 3H). |
| 26 | N-[(6-methyl- 3-pyridyl) methyl]-1-(p- tolylsulfonyl) pyrrole-3- carboxamide | 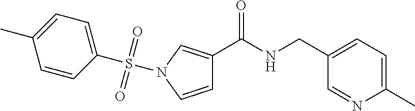 | A | 370.0 | 2.01 | ¹H NMR (CDCl₃ 400 MHz): δ 8.40 (d, 1H), 7.76 (d, 2H), 7.65 (t, 1H), 7.57 (dd, 1H), 7.31 (d, 2H), 7.13-7.10 (m, 2H), 6.50 (dd, |
| | | | | | | 1H), 6.21 (brs, |
| | | | | | | 1H), 4.52 (d, 2H), |
| | | | | | | 2.53 (s, 3H), 2.41 |
| | | | | | | (s, 3H). |
| 27 | N-[(5-methyl- 2-pyridyl) methyl]-1-(p- tolylsulfonyl) pyrrole-3- carboxamide | 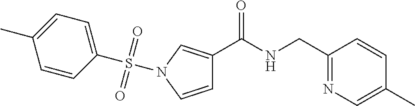 | A | 370.1 | 1.71 | ¹H NMR (CDCl₃ 400 MHz): δ 8.34 (s, 1H), 7.76 (d, 2H), 7.68 (t, 1H), 7.45 (dd, 1H), 7.29 (d, 2H), 7.13-7.11 (m, 3H), 6.57 (dd, 2.0 |
| | | | | | | Hz, 1H), 4.60 (d, |
| | | | | | | 2H), 2.39 (s, 3H), |
| | | | | | | 2.31 (s, 3H). |
| 28 | N-[(5- methylpyrazin- 2-yl)methyl]- 1-(o- tolylsulfonyl) pyrrole-3- carboxamide | 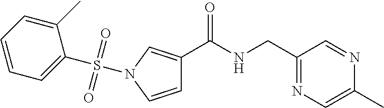 [SMILES: Cc1cnc(CNC(=O)c2ccn(S(=O)(=O)c3ccccc3C)c2)cn1] | A | 371.1 | 2.11 | ¹H NMR (DMSO-d⁶ 400 MHz): δ 8.87 (t, 1H), 8.46 (s, 2H), 7.91 (s, 1H), 7.88 (d, 1H), 7.69 (t, 1H), 7.54-7.48 (m, 2H), 7.39 (t, |
| | | | | | | 1H), 6.75 (s, 1H), |
| | | | | | | 4.48 (d, 2H), 2.52 |
| | | | | | | (s, 3H), 2.46 (s, |
| | | | | | | 3H). |
| 29 | 1-(p- tolylsulfonyl)- N-(pyrazin-2- ylmethyl) pyrrole-3- carboxamide | 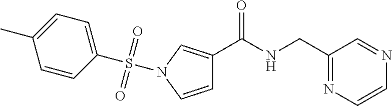 [SMILES: Cc1ccc(S(=O)(=O)n2ccc(C(=O)NCc3cnccn3)c2)cc1] | A | 357.1 | 1.91 | ¹H NMR (CDCl₃ 400 MHz): δ 8.62 (s, 1H), 8.51-8.49 (m, 2H), 7.76 (d, 2H), 7.68 (t, 1H), 7.30 (d, 2H), 7.14 (t, 1H), 6.87 (brs, 1H), 6.56 (t, 1H), |
| | | | | | | 4.71 (d, 2H), 2.39 |
| | | | | | | (s, 3H). |
| 30 | N-[(5- methylpyrazin- 2-yl)methyl]- 1-(m- tolylsulfonyl) pyrrole-3- carboxamide | 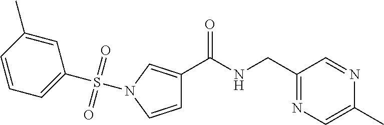 [SMILES: Cc1cccc(S(=O)(=O)n2ccc(C(=O)NCc3cnc(C)cn3)c2)c1] | A | 371.1 | 2.14 | ¹H NMR (DMSO-d⁶ 400 MHz): δ 8.78 (t, 1H), 8.41 (s, 2H), 7.86 (t, 1H), 7.82 (s, 1H), 7.77 (d, 1H), 7.58-7.50 (m, 2H), 7.38- 7.36 (m, 1H), |
| | | | | | | 6.69-6.68 (m, |
| | | | | | | 1H), 4.43 (d, 2H), |
| | | | | | | 2.42 (s, 3H), 2.37 |
| | | | | | | (s, 3H). |
| 31 | N-[(5-methyl- 1,3,4- oxadiazol-2- yl)methyl]- 1-(p- tolylsulfonyl) pyrrole-3- carboxamide | 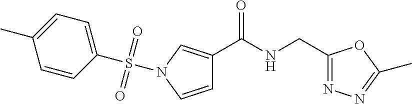 | B | 361.1 | 1.98 | ¹H NMR (DMSO-d⁶ 400 MHz): δ 8.86 (t, 1H), 7.84-7.85 (m, 3H), 7.44 (d, 2H), 7.37 (t, 1H), 6.66 (q, 1H), 4.53 (d, 2H), 2.41 (s, 3H), 2.35 (s, 3H). |
| 32 | N-[(5- methylisoxazol- 3-yl)methyl]- 1-(p- tolylsulfonyl) pyrrole-3- carboxamide | 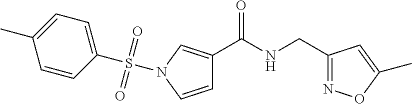 | A | 360.1 | 2.31 | ¹H NMR (DMSO-d⁶ 400 MHz): δ 8.71 (t, 1H), 7.88-7.84 (m, 3H), 7.44 (d, 2H), 7.36 (t, 1H), 6.67 (q, 1H), 6.07 |
| | | | | | | (s, 1H), 4.32 (d, |
| | | | | | | 2H), 2.35 (s, 3H), |
| | | | | | | 2.31 (s, 3H). |
| 33 | N-[(5- methyloxazol- 2-yl)methyl]- 1-(p- tolylsulfonyl) pyrrole-3- carboxamide | 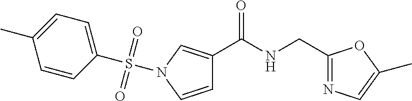 | A | 360.1 | 2.23 | ¹H NMR (CDCl₃ 400 MHz): δ 7.78 (d, 2H), 7.70 (t, 1H), 7.32 (d, 2H), 7.16-7.14 (m, 1H), 6.66 (d, 1H), 6.57-6.56 |
| | | | | | | (m, 1H), 6.48 |
| | | | | | | (brs, 1H), 4.63 |
| | | | | | | (d, 2H), 2.43 (s, |
| | | | | | | 3H), 2.30 (s, 3H). |
| 34 | N-[(4- methylthiazol- 2-yl)methyl]- 1-(p- tolylsulfonyl) pyrrole-3- carboxamide | 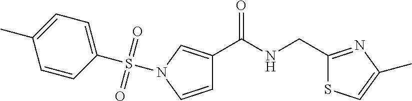 | A | 376.1 | 2.25 | ¹H NMR (CDCl₃ 400 MHz): δ 7.78 (d, 2H), 7.68 (t, 1H), 7.32 (d, 2H), 7.14 (dd, 1H), 6.82 (d, 1H), 6.60 (brs, 1H), 6.54 (dd, 1H), 4.80 (d, 2H), 2.42 (s, 3H), 2.41 (s, 3H). |
| 35 | N-[(3- methyl- isoxazol-5- yl)methyl]- 1-(p- tolylsulfonyl) pyrrole-3- carboxamide | 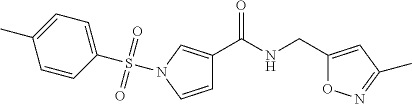 | A | 360.1 | 2.27 | ¹H NMR (DMSO-d⁶ 400 MHz): δ 8.79 (t, 1H), 7.88-7.85 (m, 3H), 7.44 (d, 2H), 7.37 (t, 1H), 6.67 (q, 1H), 6.14 (s, 1H), 4.42 (d, |
| | | | | | | 2H), 2.35 (s, 3H), |
| | | | | | | 2.14 (s, 3H). |
| 36 | N-[(1- methylpyrazol- 3-yl)methyl]- 1-(p- tolylsulfonyl) pyrrole-3- carboxamide | 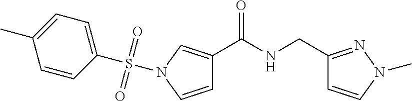 | A | 359.1 | 2.13 | ¹H-NMR (CDCl₃ 400 MHz): δ 7.75 (t, 2H), 7.63 (d, 1H), 7.30-7.27 (m, 3H), 7.10 (t, 1H), 6.51 (t, 1H), 6.31 (brs, 1H), |
| | | | | | | 6.16 (d, 1H), |
| | | | | | | 4.52 (d, 2H), |
| | | | | | | 3.85 (s, 3H), 2.40 |
| | | | | | | (s, 3H). |
| 37 | N-[(1- methylpyrazol- 4-yl)methyl]- 1-(p- tolylsulfonyl) pyrrole-3- carboxamide | 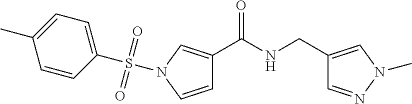 | B | 359.1 | 2.04 | ¹H NMR (DMSO-d⁶ 400 MHz): δ 8.43 (t, 1H), 7.86-7.81 (m, 3H), 7.52 (s, 1H), 7.43 (d, 2H), 7.33 (t, 1H), 7.27 (s, 1H), 6.51 (q, |
| | | | | | | 1H), 4.15 (d, 2H), |
| | | | | | | 3.73 (s, 3H), |
| | | | | | | 2.35 (s, 3H). |
| 38 | N-[(2- methyloxazol- 5-yl)methyl]- 1-(p- tolylsulfonyl) pyrrole-3- carboxamide | 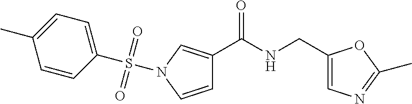 | A | 360.1 | 2.13 | ¹H NMR (CDCl₃ 400 MHz): δ 7.77 (d, 2H), 7.65 (t, 1H), 7.31 (d, 2H), 7.14 (dd, 1H), 6.84 (s, 1H), 6.51 (q, 1H), 6.07 (t, 1H), 4.55 |
| | | | | | | (d, 2H), 2.42 (s, |
| | | | | | | 3H), 2.42 (s, 3H). |
| 39 | N-[(5- methylthiazol- 2-yl)methyl]- 1-(p- tolylsulfonyl) pyrrole-3- carboxamide | 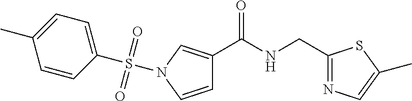 | A | 375.9 | 2.12 | ¹H NMR (CDCl₃ 400 MHz): δ 7.75 (d, 2H), 7.67-7.66 (m, 1H), 7.30- 7.28 (m, 3H), 7.12-7.11 (m, 1H), 6.71 (brs, |
| | | | | | | 1H), 6.53-6.52 |
| | | | | | | (m, 1H), 4.74 (d, |
| | | | | | | 2H), 2.41 (s, 3H), |
| | | | | | | 2.39 (s, 3H). |
| 40 | N-[(1-methyl- imidazol-4- yl)methyl]- 1-(p- tolylsulfonyl) pyrrole-3- carboxamide | 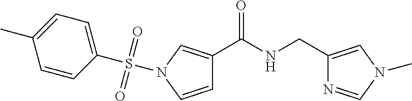 | A | 359.1 | 1.81 | ¹H NMR (CDCl₃ 400 MHz): δ 7.71 (d, 2H), 7.63- 7.62 (m, 1H), 7.32 (s, 1H), 7.26 (d, 2H), 7.07- 7.06 (m, 1H), |
| | | | | | | 6.93 (brs, 1H), |
| | | | | | | 6.84 (s, 1H), |
| | | | | | | 6.53-6.51 (m, |
| | | | | | | 1H), 4.41 (d, |
| | | | | | | 2H), 3.61 (s, |
| | | | | | | 3H), 2.38 (s, |
| | | | | | | 3H). |
| 41 | N-[(1- methyltriazol- 4-yl)methyl]- 1-(p- tolylsulfonyl) pyrrole-3- carboxamide | 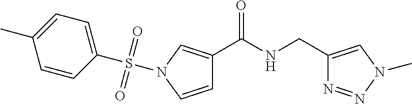 | B | 360.1 | 1.97 | ¹H NMR (CDCl₃ 400 MHz): δ 7.76 (d, 2H), 7.67 (t, 1H), 7.58 (s, 1H), 7.30 (d, 2H), 7.12 (t, 1H), 6.74 (brs, 1H), 6.52 (dd, |
| | | | | | | 1H), 4.60 (d, 2H), |
| | | | | | | 4.06 (s, 3H), 2.41 |
| | | | | | | (s, 3H). |
| 42 | N-[(1-methyl- 1,2,4-triazol- 3-yl)methyl]- 1-(p- tolylsulfonyl) pyrrole-3- carboxamide | 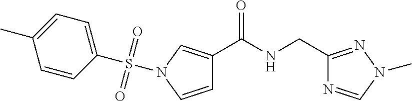 | B | 360.1 | 1.88 | ¹H NMR (DMSO-d⁶ 400 MHz): δ 8.62 (t, 1H), 8.31 (s, 1H), 7.87-7.83 (m, 3H), 7.44 (d, 2H), 7.34 (t, 1H), |
| | | | | | | 6.68-6.67 (m, |
| | | | | | | 1H), 4.34 (d, |
| | | | | | | 2H), 3.76 (s, 3H), |
| | | | | | | 2.35 (s, 3H). |
| 43 | N-[(3-methyl- 1,2,4- oxadiazol- 5-yl)methyl]- 1-(p- tolylsulfonyl) pyrrole-3- carboxamide | 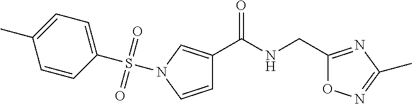 | A | 360.9 | 2.02 | ¹H NMR (DMSO-d⁶ 400 MHz): δ 8.99 (t, 1H), 7.90-7.88 (m, 3H), 7.46 (d, 2H), 7.40 (t, 1H), 6.69 (q, 1H), 4.59 (d, 2H), 2.37 (s, |
| | | | | | | 3H), 2.28 (s, 3H). |
| 47 | 1-(4-methyl- benzene-1- sulfonyl)-N- [(2-methyl- 1,3-oxazol- 4-yl)methyl]- 1H-pyrrole-3- carboxamide | 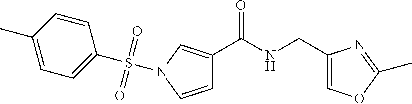 | A | 360 | 2.06 | ¹H NMR (DMSO-d⁶ 400 MHz): δ 8.53 (t, 1H), 7.90-7.86 (m, 3H), 7.74 (s, 1H), 7.45 (d, 2H), 7.36-7.34 (m, 1H), 6.69- |
| | | | | | | 6.67 (m, 1H), |
| | | | | | | 4.18 (d, 2H), 2.35 |
| | | | | | | (s, 3H), 2.33 (s, |
| | | | | | | 3H). |
| 48 | 1-(benzene- sulfonyl)-N- [(5-methyl- pyrazin-2- yl)methyl]- 1H-pyrrole-3- carboxamide | 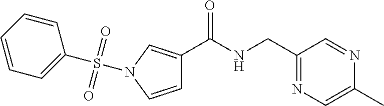 [SMILES: Cc1cnc(CNC(=O)c2ccn(S(=O)(=O)c3ccccc3)c2)cn1] | A | 357.2 | 1.95 | ¹H NMR (CDCl₃ 400 MHz): δ 8.52 (s, 1H), 8.39 (s, 1H), 7.90 (d, 2H), 7.72-7.71 (m, 1H), 7.67-7.63 (m, 1H), 7.56- 7.52 (m, 2H), |
| | | | | | | 7.18-7.17 (m, |
| | | | | | | 1H), 6.86 (brs, |
| | | | | | | 1H), 6.59-6.58 |
| | | | | | | (m, 1H), 4.68 (d, |
| | | | | | | 2H), 2.57 (s, 3H). |
| 49 | 1-(4-methyl- benzene-1- sulfonyl)-N- [(1,3-thiazol- 4-yl)methyl]- 1H-pyrrole-3- carboxamide | 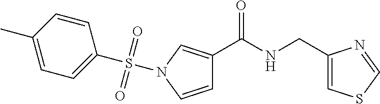 [SMILES: Cc1ccc(S(=O)(=O)n2ccc(C(=O)NCc3cscn3)c2)cc1] | A | 362 | 2.01 | ¹H NMR (CDCl₃ 400 MHz): δ 8.79 (s, 1H), 7.77 (d, 2H), 7.66 (s, 1H), 7.32-7.27 (m, 3H), 7.13-7.12 (m, 1H), 6.61 (s, |
| | | | | | | 1H), 6.53-6.52 |
| | | | | | | (m, 1H), 4.70 |
| | | | | | | (d, 2H), 2.41 (s, |
| | | | | | | 3H). |
| 50 | 1-(4-methyl- benzene-1- sulfonyl)-N- [(1,3-oxazol- 5-yl)methyl]- 1H-pyrrole-3- carboxamide | 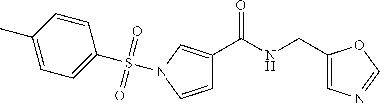 [SMILES: Cc1ccc(S(=O)(=O)n2ccc(C(=O)NCc3cnco3)c2)cc1] | B | 346.1 | 2.04 | ¹H NMR (CDCl₃ 400 MHz): δ 7.81 (s, 1H), 7.77 (d, 2H), 7.67-7.66 (m, 1H), 7.32 (d, 2H), 7.15-7.14 (m, 1H), 7.01 (s, |
| | | | | | | 1H), 6.51-6.50 |
| | | | | | | (m, 1H), 6.13 |
| | | | | | | (brs, 1H), 4.63 |
| | | | | | | (d, 2H), 2.42 (s, |
| | | | | | | 3H). |
| 51 | 1-(4-methyl- benzene-1- sulfonyl)-N- [(1,3-thiazol- 2-yl)methyl]- 1H-pyrrole-3- carboxamide | 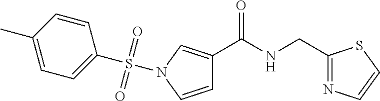 [SMILES: Cc1ccc(S(=O)(=O)n2ccc(C(=O)NCc3nccs3)c2)cc1] | A | 362.1 | 2.19 | ¹H NMR (CDCl₃ 400 MHz): δ 7.76-7.68 (m, 4H), 7.28 (m, 3H), 7.12 (s, 2H), 6.57 (s, 1H), 4.84 (s, |
| | | | | | | 2H), 2.40 (s, |
| | | | | | | 3H). |
| 52 | 1-(4-methyl- benzene-1- sulfonyl)-N- [(1,2-oxazol- 3-yl)methyl]- 1H-pyrrole-3- carboxamide | 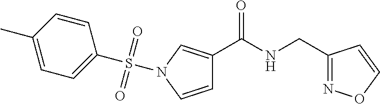 [SMILES: Cc1ccc(S(=O)(=O)n2ccc(C(=O)NCc3ccon3)c2)cc1] | A | 346.1 | 2.19 | ¹H NMR (CDCl₃ 400 MHz): δ 8.36 (s, 1H), 7.78 (d, 2H), 7.68 (d, 1H), 7.31 (d, 2H), 7.15-7.14 (m, 1H), 6.53 (t, 1H), |
| | | | | | | 6.44 (s, 1H), 6.38 |
| | | | | | | (d, 1H), 4.66 (d, |
| | | | | | | 2H), 2.41 (s, |
| | | | | | | 3H). |
| 53 | 1-(4-methyl- benzene-1- sulfonyl)-N- [(1,2-oxazol- 5-yl)methyl]- 1H-pyrrole-3- carboxamide | 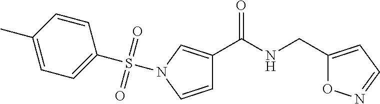 [SMILES: Cc1ccc(S(=O)(=O)n2ccc(C(=O)NCc3ccno3)c2)cc1] | A | 346.1 | 2.19 | ¹H NMR (CDCl₃ 400 MHz): δ 8.18 (s, 1H), 7.77 (d, 2H), 7.67 (t, 1H), 7.32 (d, 2H), 7.16-7.14 (m, 1H), 6.52 (d, 1H), 6.34 (br s, |
| | | | | | | 1H), 6.23 (s, 1H), |
| | | | | | | 4.70 (d, 2H), 2.42 |
| | | | | | | (s, 3H). |
| 54 | 1-(4-methyl- benzene-1- sulfonyl)-N- [(1,3-oxazol- 4-yl)methyl]- 1H-pyrrole-3- carboxamide | 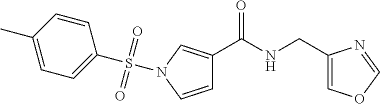 [SMILES: Cc1ccc(S(=O)(=O)n2ccc(C(=O)NCc3cocn3)c2)cc1] | A | 346 | 1.95 | ¹H NMR (CDCl₃ 400 MHz): δ 7.87 (s, 1H), 7.77 (d, 2H), 7.65-7.64 (m, 2H), 7.31 (d, 2H), 7.13-7.12 (m, 1H), 6.52- |
| | | | | | | 6.51 (m, 1H), |
| | | | | | | 6.40 (brs, 1H), |
| | | | | | | 4.48 (d, 2H), |
| | | | | | | 2.41 (s, 3H). |
| 55 | 1-(4-methyl- benzene-1- sulfonyl)-N- [(1,2-thiazol- 4-yl)methyl]- 1H-pyrrole-3- carboxamide | 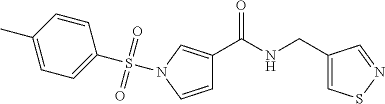 [SMILES: Cc1ccc(S(=O)(=O)n2ccc(C(=O)NCc3cnsc3)c2)cc1] | A | 362.1 | 2.24 | ¹H NMR (CDCl₃ 400 MHz): δ 8.53 (s, 1H), 8.47 (s, 1H), 7.77 (d, 2H), 7.65 (t, 1H), 7.32 (d, 2H), 7.15-7.14 (m, 1H), 6.49- |
| | | | | | | 6.48 (m, 1H), |
| | | | | | | 6.14 (br s, 1H), |
| | | | | | | 4.64 (d, 2H), 2.41 |
| | | | | | | (s, 3H). |
| 56 | 1-(4-methyl- benzene-1- sulfonyl)-N- [(1,3,4- thiadiazol-2- yl)methyl]- 1H-pyrrole-3- carboxamide | 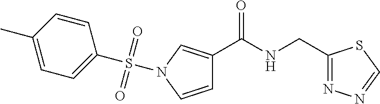 [SMILES: Cc1ccc(S(=O)(=O)n2ccc(C(=O)NCc3nncs3)c2)cc1] | A | 363 | 2.26 | ¹H NMR (CDCl₃ 400 MHz): δ 9.11 (s, 1H), 7.79-7.74 (m, 3H), 7.31 (d, 2H), 7.15-7.14 (m, 1H), 7.03 (s, 1H), 6.58-6.57 (m, 1H), 5.00 (d, |
| | | | | | | 2H), 2.41 (s, 3H). |
| 57 | 1-(4-methyl- benzene-1- sulfonyl)-N- [(1,2,4- oxadiazol-3- yl)methyl]- 1H-pyrrole-3- carboxamide | 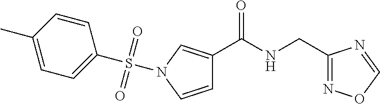 [SMILES: Cc1ccc(S(=O)(=O)n2ccc(C(=O)NCc3ncon3)c2)cc1] | A | 347.1 | 2.1 | ¹H NMR (CDCl₃ 400 MHz): δ 8.70 (s, 1H), 7.77 (d, 2H), 7.70 (s, 1H), 7.31 (d, 2H), 7.14 (t, 1H), 6.56 (s, 1H), 6.51 (br s, 1H), 4.77 (d, 2H), |
| | | | | | | 2.41 (s, 3H). |
| 58 | 1-(4-methyl- benzene-1- sulfonyl)-N- [(pyrimidin- 5-yl)methyl]- 1H-pyrrole-3- carboxamide | 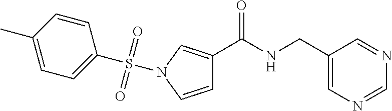 [SMILES: Cc1ccc(S(=O)(=O)n2ccc(C(=O)NCc3cncnc3)c2)cc1] | A | 357.1 | 2.01 | ¹H NMR (DMSO-d⁶ 400 MHz): δ 9.14 (s, 1H), 8.50 (t, 1H), 8.79 (s, 2H), 7.97 (s, 1H), 7.95 (s, 2H), 7.54 (d, 2H), 7.46 (t, 1H), |
| | | | | | | 6.76 (t, 1H), 3.76 |
| | | | | | | (d, 2H), 2.45 (s, |
| | | | | | | 3H). |
| 59 | 1-(2-fluoro- benzene-1- sulfonyl)-N- [(pyrazin-2- yl)methyl]- 1H-pyrrole-3- carboxamide | 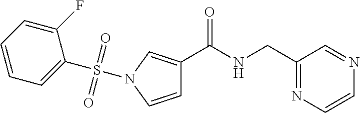 [SMILES: O=C(NCc1cnccn1)c1ccn(S(=O)(=O)c2ccccc2F)c1] | B | 361 | 1.52 | ¹H NMR (DMSO-d⁶ 400 MHz): δ 8.95 (t, 1H), 8.60 (s, 1H), 8.57 (d, 1H), 8.52 (d, 1H), 7.80 (t, 1H), 7.94 (s, 1H), 7.89-7.86 |
| | | | | | | (m, 1H), 7.57- |
| | | | | | | 7.50 (m, 2H), |
| | | | | | | 7.38 (s, 1H), 6.78 |
| | | | | | | (t, 1H), 4.53 (d, |
| | | | | | | 2H). |
| 60 | 1-(3-methyl- benzene-1- sulfonyl)-N- [(1-methyl- 1H-pyrazol- 3-yl)methyl]- 1H-pyrrole-3- carboxamide | 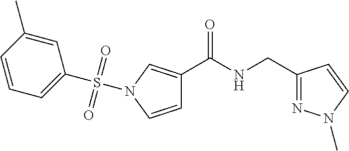 [SMILES: Cc1cccc(S(=O)(=O)n2ccc(C(=O)NCc3ccn(C)n3)c2)c1] | A | 359 | 2.01 | ¹H-NMR (CDCl₃ 400 MHz): δ 7.68-7.66 (m, 3H), 7.41-7.39 (m, 2H), 7.28- 7.27 (m, 1H), 7.13 (t, 1H), 6.53 (t, 1H), 6.39 (s, 1H), 6.17 (d, 1H), 4.53 (d, |
| | | | | | | 2H), 3.86 (s, |
| | | | | | | 3H), 2.41 (s, |
| | | | | | | 3H). |
| 61 | 1-(3-methyl- benzene-1- sulfonyl)-N- [(3-methyl- 1,2,4- oxadiazol- 5-yl)methyl]- 1H-pyrrole-3- carboxamide | 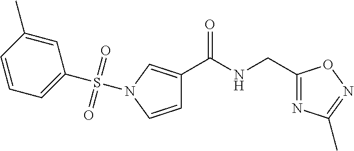 [SMILES: Cc1cccc(S(=O)(=O)n2ccc(C(=O)NCc3nc(C)no3)c2)c1] | A | 361 | 2.08 | ¹H-NMR (CDCl₃ 400 MHz): δ 7.71-7.68 (m, 3H), 7.42-7.40 (m, 2H), 7.16- 7.15 (m, 1H), 6.56-6.55 (m, 1H), 6.44 (s, 1H), 4.76 (d, 2H), 2.41 (s, 3H), 2.37 (s, |
| | | | | | | 3H). |
| 62 | 1-(3-methyl- benzene-1- sulfonyl)-N- [(5-methyl- pyrimidin- 2-yl)methyl]- 1H-pyrrole-3- carboxamide | 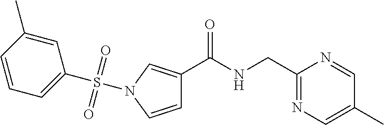 [SMILES: Cc1cnc(CNC(=O)c2ccn(S(=O)(=O)c3cccc(C)c3)c2)nc1] | A | 371 | 2.01 | ¹H-NMR (CDCl₃ 400 MHz): δ 8.53 (s, 2H), 7.72-7.68 (m, 3H), 7.40- 7.36 (m, 2H), 7.16-7.14 (m, 2H), 6.63-6.61 (m, 1H), 4.75 (d, 2H), 2.40 (s, |
| | | | | | | 3H), 2.30 (s, 3H). |
| 63 | 1-(4-fluoro- benzene-1- sulfonyl)-N- [(1-methyl- 1H-pyrazol- 3-yl)methyl)- 1H-pyrrole-3- carboxamide | 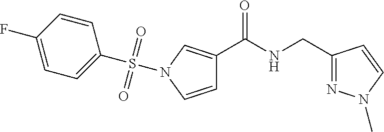 [SMILES: Cn1ccc(CNC(=O)c2ccn(S(=O)(=O)c3ccc(F)cc3)c2)n1] | A | 363.1 | 2.04 | ¹H NMR (DMSO-d⁶ 400 MHz): δ 8.57 (t, 1H), 8.13-8.09 (m, 2H), 7.91 (t, 1H), 7.56-7.50 (m, 3H), 7.41- 7.40 (m, 1H), 6.74-6.73 (m, |
| | | | | | | 1H), 6.07 (d, 1H), |
| | | | | | | 4.30 (d, 2H), 3.76 |
| | | | | | | (s, 3H). |
| 64 | 1-(4-fluoro- benzene-1- sulfonyl)-N- [(pyrazin-2- yl)methyl]- 1H-pyrrole-3- carboxamide | 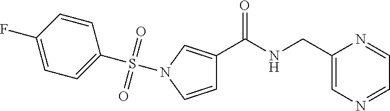 [SMILES: O=C(NCc1cnccn1)c1ccn(S(=O)(=O)c2ccc(F)cc2)c1] | B | 361.1 | 1.93 | ¹H NMR (DMSO-d⁶ 400 MHz): δ 8.89 (t, 1H), 8.60 (s, 1H), 8.58-8.57 (m, 1H), 8.52 (d, 1H), 8.15-8.11 (m, 2H), 7.94 (t, |
| | | | | | | 1H), 7.53 (t, 2H), |
| | | | | | | 7.44 (t, 1H), |
| | | | | | | 6.75-6.74 (m, |
| | | | | | | 1H), 4.52 (d, |
| | | | | | | 2H). |
| 65 | 1-(4-fluoro- benzene-1- sulfonyl)-N- [(3-methyl- 1,2,4- oxadiazol-5- yl)methyl]- 1H-pyrrole-3- carboxamide | 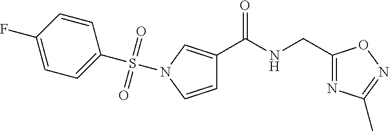 [SMILES: Cc1noc(CNC(=O)c2ccn(S(=O)(=O)c3ccc(F)cc3)c2)n1] | A | 365.1 | 2.09 | ¹H NMR (DMSO-d⁶ 400 MHz): δ 9.02 (t, 1H), 8.16-8.12 (m, 2H), 7.94 (t, 1H), 7.54 (t, 2H), 7.47-7.46 (m, 1H), 6.74-6.73 (m, 1H), 4.61 (d, |
| | | | | | | 2H), 2.30 (s, |
| | | | | | | 3H). |
| 66 | 1-(4-methoxy- benzene-1- sulfonyl)-N- [(1-methyl- 1H-pyrazol- 3-yl)methyl]- 1H-pyrrole-3- carboxamide | 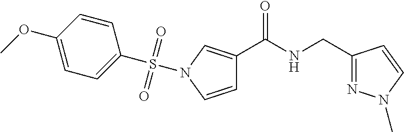 [SMILES: COc1ccc(S(=O)(=O)n2ccc(C(=O)NCc3ccn(C)n3)c2)cc1] | A | 375.1 | 2.06 | ¹H NMR (DMSO-d⁶ 400 MHz): δ 8.54 (t, 1H), 7.94 (d, 2H), 7.87 (t, 1H), 7.55 (d, 1H), 7.36-7.34 (m, 1H), 7.16 (d, 2H), 6.70-6.69 |
| | | | | | | (m, 1H), 6.07 (d, |
| | | | | | | 1H), 4.29 (d, |
| | | | | | | 2H), 3.84 (s, |
| | | | | | | 3H), 3.76 (s, 3H). |
| 67 | 1-(4-methoxy- benzene-1- sulfonyl)-N- [(pyrazin-2- yl)methyl]- 1H-pyrrole-3- carboxamide | 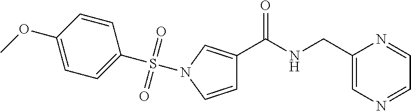 [SMILES: COc1ccc(S(=O)(=O)n2ccc(C(=O)NCc3cnccn3)c2)cc1] | B | 373.1 | 1.9 | ¹H NMR (DMSO-d⁶ 400 MHz): δ 8.74 (t, 1H), 8.59 (s, 1H), 8.58-8.56 (m, 1H), 8.52 (d, 1H), 7.96 (d, 2H), 7.89 (t, 1H), 7.38 |
| | | | | | | (t, 1H), 7.17 (d, |
| | | | | | | 2H), 6.72-6.70 |
| | | | | | | (m, 1H), 4.51 (d, |
| | | | | | | 2H), 3.85 (s, 3H). |
| 68 | 1-(4-methoxy- benzene-1- sulfonyl)-N- [(3-methyl- 1,2,4- oxadiazol-5- yl)methyl]- 1H-pyrrole-3- carboxamide | 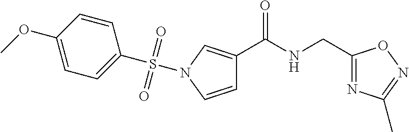 [SMILES: COc1ccc(S(=O)(=O)n2ccc(C(=O)NCc3nc(C)no3)c2)cc1] | B | 377.1 | 2.03 | ¹H NMR (DMSO-d⁶ 400 MHz): δ 9.0 (t, 1H), 7.98-7.95 (m, 2H), 7.90 (t, 1H), 7.41-7.40 (m, 1H), 7.19- 7.16 (m, 2H), 6.69 (t, 1H), 4.60 |
| | | | | | | (d, 2H), 3.85 (s, |
| | | | | | | 3H), 2.29 (s, |
| | | | | | | 3H). |
| 69 | 1-(4-methoxy- benzene-1- sulfonyl)-N- [(1-methyl- 1H-pyrazol- 4-yl)methyl]- 1H-pyrrole-3- carboxamide | 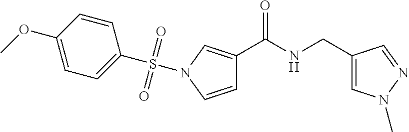 [SMILES: COc1ccc(S(=O)(=O)n2ccc(C(=O)NCc3cnn(C)c3)c2)cc1] | A | 375.1 | 2.01 | ¹H NMR (DMSO-d⁶ 400 MHz): δ 8.45 (t, 1H), 7.93 (d, 2H), 7.84 (t, 1H), 7.55 (s, 1H), 7.34 (t, 1H), 7.03 (s, 1H), 7.16 (d, 2H), 6.68-6.66 |
| | | | | | | (m, 1H), 4.18 |
| | | | | | | (d, 2H), 3.84 (s, |
| | | | | | | 3H), 3.76 (s, |
| | | | | | | 3H). |
| 70 | 1-(4-methoxy- benzene-1- sulfonyl)-N- [(5-methyl- pyridin-2- yl)methyl]- 1H-pyrrole-3- | 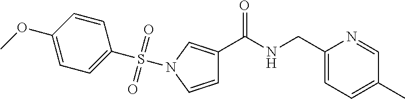 | A | 386.1 | 2 | ¹H NMR (DMSO-d⁶ 400 MHz): δ 8.76 (t, 1H), 8.32 (s, 1H), 7.96 (d, 2H), 7.89 (t, 1H), 7.55- 7.53 (m, 1H), |
| | carboxamide | | | | | 7.38 (t, 1H), 7.17 |
| | | | | | | (m, 3H), 6.73- |
| | | | | | | 6.72 (m, 1H), |
| | | | | | | 4.42 (d, 2H), 3.85 |
| | | | | | | (s, 3H), 2.26 (s, |
| | | | | | | 3H). |
| 71 | 1-(4-methoxy- benzene-1- sulfonyl)-N- [(3-methyl- 1,2-oxazol- 5-yl)methyl]- 1H-pyrrole-3- carboxamide | 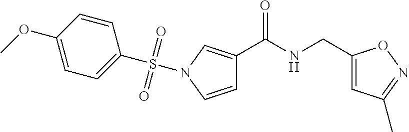 [SMILES: COc1ccc(S(=O)(=O)n2ccc(C(=O)NCc3cc(C)no3)c2)cc1] | A | 376.1 | 2.4 | ¹H NMR (DMSO-d⁶ 400 MHz): δ 8.81 (m, 1H), 7.95 (d, 2H), 7.88 (s, 1H), 7.38 (t, 1H), 7.17 (d, 2H), 6.68 (d, 1H), 6.17 (s, 1H), 4.45 (d, 2H), |
| | | | | | | 3.85 (s, 3H), 2.17 |
| | | | | | | (s, 3H). |
| 72 | 1-(4-methoxy- benzene-1- sulfonyl)-N- [(5-methyl- pyrimidin- 2-yl)methyl]- 1H-pyrrole-3- | 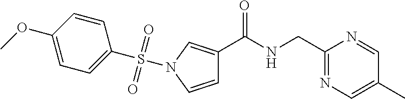 | A | 387.1 | 2.05 | ¹H NMR (DMSO-d⁶ 400 MHz): δ 8.74 (t, 1H), 8.58 (s, 2H), 7.97-7.94 (m, 2H), 7.88 (s, 1H), 7.38 (t, 1H), |
| | carboxamide | | | | | 7.18-7.16 (m, |
| | | | | | | 2H), 6.72-6.71 |
| | | | | | | (m, 1H), 4.51 (d, |
| | | | | | | 2H), 3.85 (s, 3H), |
| | | | | | | 2.23 (s, 3H). |
| 73 | 1-(4-methoxy- benzene-1- sulfonyl)-N- [(5-methyl- 1,3-oxazol- 2-yl)methyl]- 1H-pyrrole-3- | 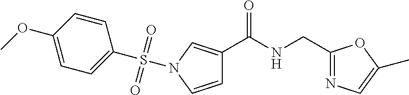 | A | 376.1 | 2.14 | ¹H NMR (DMSO-d⁶ 400 MHz): δ 8.79 (t, 1H), 7.97-7.94 (d, 2H), 7.89 (t, 1H), 7.39-7.37 (m, 1H), 7.17 (d, |
| | carboxamide | | | | | 2H), 6.71 (s, 1H), |
| | | | | | | 6.70 (d, 1H), 4.41 |
| | | | | | | (d, 2H), 3.85 (s, |
| | | | | | | 3H), 2.07 (s, |
| | | | | | | 3H). |
| 74 | 1-(2-methyl- benzene-1- sulfonyl)-N- [(1-methyl- 1H-pyrazol- 3-yl)methyl]- 1H-pyrrole-3- | 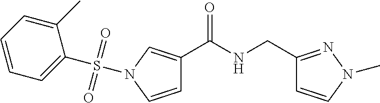 [SMILES: Cc1ccccc1S(=O)(=O)n1ccc(C(=O)NCc2ccn(C)n2)c1] | A | 359 | 1.98 | ¹H NMR (CDCl₃ 400 MHz): δ 7.80 (d, 1H), 7.61 (d, 1H), 7.50 (t, 1H), 7.33 (t, 1H), 7.29-7.27 (m, 2H), 7.13-7.11 |
| | carboxamide | | | | | (m, 1H), 6.56- |
| | | | | | | 6.54 (m, 1H), |
| | | | | | | 6.39 (s, 1H), |
| | | | | | | 6.17 (s, 1H), |
| | | | | | | 4.53 (d, 2H), |
| | | | | | | 3.85 (s, 3H), |
| | | | | | | 2.53 (s, 3H). |
| 75 | 1-(2-methyl- benzene-1- sulfonyl)-N- [(3-methyl- 1,2,4- oxadiazol- 5-yl)methyl]- | 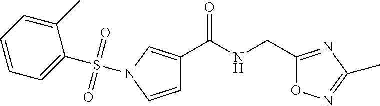 [SMILES: Cc1noc(CNC(=O)c2ccn(S(=O)(=O)c3ccccc3C)c2)n1] | A | 361.1 | 2.16 | ¹H NMR (CDCl₃ 400 MHz): δ 7.80 (d, 1H), 7.63- 7.62 (m, 1H), 7.47 (t, 1H), 7.31 (t, 1H), 7.25 (d, 1H), 7.11-7.09 |
| | 1H-pyrrole-3- | | | | | (m, 1H), 6.54- |
| | carboxamide | | | | | 6.52 (m, 1H), |
| | | | | | | 6.45 (s, 1H), |
| | | | | | | 4.72 (d, 2H), |
| | | | | | | 2.48 (s, 3H), |
| | | | | | | 2.32 (s, 3H). |
| 76 | 1-(2-methyl- benzene-1- sulfonyl)-N- [(1-methyl- 1H-pyrazol- 4-yl)methyl]- 1H-pyrrole-3- carboxamide | 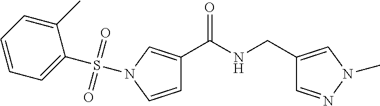 [SMILES: Cc1ccccc1S(=O)(=O)n1ccc(C(=O)NCc2cnn(C)c2)c1] | A | 359.1 | 2.06 | ¹H NMR (CDCl₃ 400 MHz): δ 7.83 (d, 1H), 7.59 (s, 1H), 7.54-7.52 (m, 1H), 7.43 (s, 1H), 7.38-7.35 (m, 2 H), 7.29 (d, 1H), 7.14 (s, |
| | | | | | | 1H), 6.50 (s, |
| | | | | | | 1H), 6.00 (s, |
| | | | | | | 1H), 4.40 (d, |
| | | | | | | 2H), 3.86 (s, |
| | | | | | | 3H), 2.54 (s, |
| | | | | | | 3H). |
| 77 | 1-(2-methyl- benzene-1- sulfonyl)-N- [(5-methyl- pyridin-2- yl)methyl]- 1H-pyrrole-3- carboxamide | 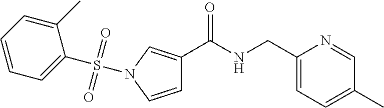 [SMILES: Cc1ccc(CNC(=O)c2ccn(S(=O)(=O)c3ccccc3C)c2)nc1] | A | 370.1 | 1.85 | ¹H NMR (CDCl₃ 400 MHz): δ 8.30 (s, 1H), 7.76 (d, 1H), 7.61 (s, 1H), 7.48-7.40 (m, 2H), 7.31-7.22 (m, 2H), 7.14- 7.09 (m, 3H), |
| | | | | | | 6.57-6.55 (m, |
| | | | | | | 1H), 4.56 (d, |
| | | | | | | 2H), 2.49 (s, 3H), |
| | | | | | | 2.26 (s, 3H). |
| 78 | 1-(2-methyl- benzene-1- sulfonyl)-N- [(3-methyl- 1,2-oxazol- 5-yl)methyl]- 1H-pyrrole-3- | 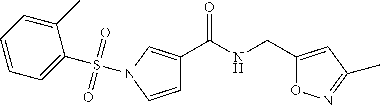 [SMILES: Cc1cc(CNC(=O)c2ccn(S(=O)(=O)c3ccccc3C)c2)on1] | A | 360.1 | 2.25 | ¹H NMR (CDCl₃ 400 MHz): δ 7.86-7.84 (m, 1H), 7.66-7.65 (m, 1H), 7.53- 7.51 (m, 1H), 7.38-7.29 (m, |
| | carboxamide | | | | | 2H), 7.15-7.12 |
| | | | | | | (m, 1H), 6.56- |
| | | | | | | 6.53 (m, 2H), |
| | | | | | | 6.06-6.03 (m, |
| | | | | | | 1H), 4.63-4.58 |
| | | | | | | (m, 2H), 2.54- |
| | | | | | | 2.50 (m, 3H), |
| | | | | | | 2.25-2.20 (m, |
| | | | | | | 3H). |
| 80 | 1-(2-methyl- benzene-1- sulfonyl)-N- [(5-methyl- 1,3-oxazol- 2-yl)methyl]- 1H-pyrrole-3- | 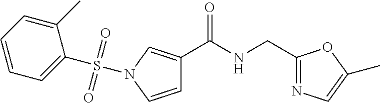 [SMILES: Cc1cnc(CNC(=O)c2ccn(S(=O)(=O)c3ccccc3C)c2)o1] | A | 360.1 | 2.2 | ¹H NMR (CDCl₃ 400 MHz): δ 7.78 (d, 1H), 7.61 (d, 1H), 7.46 (t, 1H), 7.30 (t, 1H), 7.25 (d, 1H), 7.10- 7.08 (m, 1H), |
| | carboxamide | | | | | 6.60-6.58 (m, |
| | | | | | | 1H), 6.54-6.53 |
| | | | | | | (m, 1H), 6.50- |
| | | | | | | 6.48 (m, 1H), |
| | | | | | | 4.57 (d, 2H), |
| | | | | | | 2.48 (s, 3H), |
| | | | | | | 2.23 (s, 3H). |
| 81 | 1-(4-chloro- benzene-1- sulfonyl)-N- [(pyrazin-2- yl)methyl]- 1H-pyrrole-3- carboxamide | 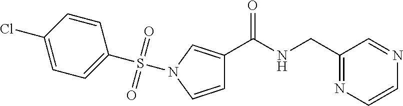 [SMILES: O=C(NCc1cnccn1)c1ccn(S(=O)(=O)c2ccc(Cl)cc2)c1] | A | 377 | 2.12 | ¹H NMR (DMSO-d⁶ 400 MHz): δ 8.88 (m, 1H), 8.60 (s, 1H), 8.57-8.56 (m, 1H), 8.52 (d, 1H), 8.04 (d, 2H), 7.94 (s, |
| | | | | | | 1H), 7.76 (d, |
| | | | | | | 2H), 7.45-7.43 |
| | | | | | | (m, 1H), 6.76- |
| | | | | | | 6.75 (m, 1H), |
| | | | | | | 4.52 (d, 2H). |
| 82 | 1-(benzene- sulfonyl)-H- [(1-methyl- 1H-pyrazol-3- yl)methyl]- 1H-pyrrole-3- carboxamide | 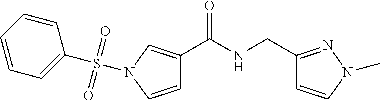 [SMILES: Cn1ccc(CNC(=O)c2ccn(S(=O)(=O)c3ccccc3)c2)n1] | A | 345.1 | 1.98 | ¹H NMR (CDCl₃ 400 MHz): δ 7.88 (d, 2H), 7.66 (t, 1H), 7.63 (t, 1H), 7.53 (t, 2H), 7.28 (d, 1H), 7.14 (t, 1H), 6.54 (dd, |
| | | | | | | 1H), 6.36 (s, 1H), |
| | | | | | | 6.18 (d, 1H), 4.54 |
| | | | | | | (d, 2H), 3.86 (s, |
| | | | | | | 3H). |
| 83 | 1-(benzene- sulfonyl)-N- [(5-methyl- pyridin-2- yl)methyl]- 1H-pyrrole-3- carboxamide | 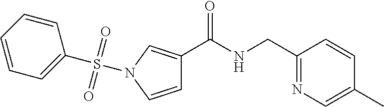 [SMILES: Cc1ccc(CNC(=O)c2ccn(S(=O)(=O)c3ccccc3)c2)nc1] | A | 356.1 | 1.72 | ¹H NMR (CDCl₃ 400 MHz): δ 8.35 (s, 1H), 7.89 (d, 2H), 7.71 (t, 1H), 7.64-7.61 (m, 1H), 7.54-7.47 (m, 3H), 7.25 (brs, 1H), 7.19 |
| | | | | | | (d, 1H), 7.15 |
| | | | | | | (dd, 1H), 6.62 |
| | | | | | | (t, 1H), 4.62 (d, |
| | | | | | | 2H), 2.32 (s, 3H). |
| 84 | 1-(benzene- sulfonyl)-N- [(3-methyl- 1,2-oxazol- 5-yl)methyl]- 1H-pyrrole-3- carboxamide | 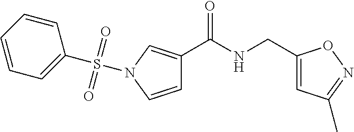 [SMILES: Cc1cc(CNC(=O)c2ccn(S(=O)(=O)c3ccccc3)c2)on1] | A | 346.1 | 2.12 | ¹H NMR (CDCl₃ 400 MHz): δ 7.89 (d, 2H), 7.69 (s, 1H), 7.65 (t, 1H), 7.53 (t, 2H), 7.16 (t, 1H), 6.53 (s, 1H), 6.34 (brs, 1H), 6.06 (s, 1H), 4.62 (d, |
| | | | | | | 2H), 2.26 (s, |
| | | | | | | 3H). |
| 85 | 1-(4-fluoro- benzene-1- sulfonyl)-N- [(6-methyl- pyridin-3- yl)methyl]- 1H-pyrrole-3- | 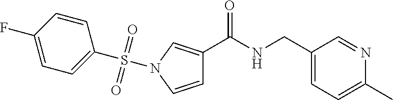 [SMILES: Cc1ccc(CNC(=O)c2ccn(S(=O)(=O)c3ccc(F)cc3)c2)cn1] | A | 374.1 | 1.73 | ¹H NMR (CDCl₃ 400 MHz): δ 8.42 (d, 1H), 7.94- 7.90 (m, 2H), 7.66 (t, 1H), 7.57 (dd, 1H), 7.23- 7.19 (m, 2H), |
| | carboxamide | | | | | 7.15-7.11 (m, |
| | | | | | | 2H), 6.51 (dd, |
| | | | | | | 1H), 6.12 (brs, |
| | | | | | | 1H), 4.53 (d, |
| | | | | | | 2H), 2.53 (s, |
| | | | | | | 3H). |
| 86 | 1-(4-methyl- benzene-1- sulfonyl)-N- [(1,3-oxazol- 2-yl)methyl]- 1H-pyrrole-3- carboxamide | 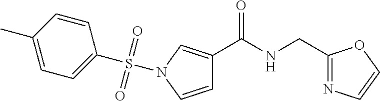 [SMILES: Cc1ccc(S(=O)(=O)n2ccc(C(=O)NCc3ncco3)c2)cc1] | A | 346.1 | 2.32 | ¹H NMR (CDCl₃ 400 MHz): δ 7.77 (d, 2H), 7.70 (s, 1H), 7.61 (s, 1H), 7.31 (d, 2H), 7.13 (s, 1H), 7.05 (s, 1H), 6.71 (brs, |
| | | | | | | 1H), 6.56 (s, 1H), |
| | | | | | | 4.68 (d, 2H), |
| | | | | | | 2.41 (s, 3H). |
| 87 | 5-fluoro-1- (4-methyl- benzene-1- sulfonyl)-N- [(5-methyl- pyrazin-2- yl)methyl]- 1H-pyrrole-3- carboxamide | 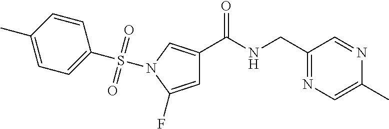 | C | 389.2 | 0.64 | ¹H NMR (600 MHz, CDCl₃) δ 8.50 (d, 1H), 8.39 (d, 1H), 7.85 (d, 2H), 7.38-7.34 (m, 3H), 6.81 (t, 1H), 5.88 (dd, 1H), 4.66 (d, 2H), |
| | | | | | | 2.56 (s, 3H), 2.44 |
| | | | | | | (s, 3H). |
| 88 | 2-fluoro-1- (4-methyl- benzene-1- sulfonyl)-N- [(5-methyl- pyrazin-2- yl)methyl]- | 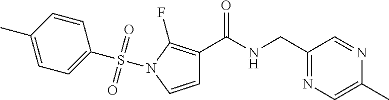 | C | 389.2 | 0.63 | ¹H NMR (600 MHz, CDCl₃) δ 8.48 (d, 1H), 8.38 (d, 1H), 7.85 (d, 2H), 7.36 (d, 2H), 6.81 (d, 1H), 6.79 (dd, |
| | 1H-pyrrole-3- | | | | | 1H), 6.49 (t, 1H), |
| | carboxamide | | | | | 4.65 (d, 2H), |
| | | | | | | 2.55 (s, 3H), |
| | | | | | | 2.45 (s, 3H). |
| 89 | N-[(5-chloro- pyrazin-2- yl)methyl]-1- (4-methyl- benzene-1- sulfonyl)-1H- pyrrole-3- | 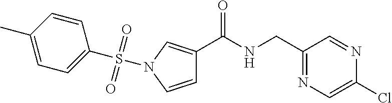 [SMILES: Cc1ccc(S(=O)(=O)n2ccc(C(=O)NCc3cnc(Cl)cn3)c2)cc1] | C | 391.2 | 0.68 | ¹H NMR (600 MHz, DMSO- d₆) δ 8.87 (t, 1H), 8.73 (d, 1H), 8.47 (d, 1H), 7.93-7.87 (m, 3H), 7.51- |
| | carboxamide | | | | | 7.45 (m, 2H), |
| | | | | | | 7.40 (m, 1H), |
| | | | | | | 6.70 (dd, 1H), |
| | | | | | | 4.52 (d, 2H), |
| | | | | | | 2.39 (s, 3H). |
| 90 | 1-(4-fluoro- 2-methyl- benzene-1- sulfonyl)-N- [(5-methyl- pyrazin-2- yl)methyl]- | 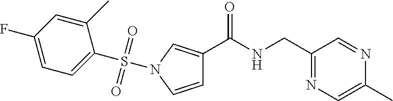 [SMILES: Cc1cnc(CNC(=O)c2ccn(S(=O)(=O)c3ccc(F)cc3C)c2)cn1] | D | 389.1 | 0.6 | ¹H NMR (600 MHz, CDCl₃) δ 8.52 (d, 1H), 8.39 (d, 1H), 7.93 (dd, 1H), 7.65 (dd, 1H), 7.15 (dd, 1H), |
| | 1H-pyrrole-3- | | | | | 7.08-7.03 (m, |
| | carboxamide | | | | | 1H), 7.01 (dd, |
| | | | | | | 1H), 6.82 (t, 1H), |
| | | | | | | 6.60 (dd, 1H), |
| | | | | | | 4.69 (d, 2H), |
| | | | | | | 2.57 (s, 3H), |
| | | | | | | 2.55 (s, 3H). |
| 91 | N-[(5- methyl- pyrazin-2- yl)methyl]-1- [4-(trifluoro- methyl) benzene-1- sulfonyl]-1H- | 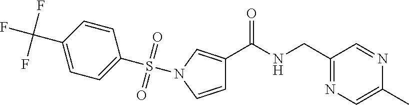 [SMILES: Cc1cnc(CNC(=O)c2ccn(S(=O)(=O)c3ccc(C(F)(F)F)cc3)c2)cn1] | C | 425.2 | 0.65 | ¹H NMR (600 MHz, CDCl₃) δ 8.51 (d, 1H), 8.38 (d, 1H), 8.02 (d, 2H), 7.80 (d, 2H), 7.71 (dd, 1H), 7.17 (dd, 1H), |
| | pyrrole-3- | | | | | 6.86 (t, 1H), |
| | carboxamide | | | | | 6.62 (dd, 1H), |
| | | | | | | 4.68 (d, 2H), |
| | | | | | | 2.56 (s, 3H). |
| 92 | 1-(3-chloro- 4-fluoro- benzene-1- sulfonyl)-N- [(5-methyl- pyrazin-2- yl)methyl]- 1H-pyrrole-3- carboxamide | 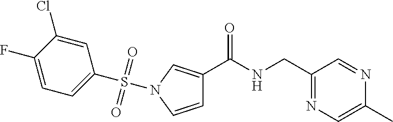 [SMILES: Cc1cnc(CNC(=O)c2ccn(S(=O)(=O)c3ccc(F)c(Cl)c3)c2)cn1] | D | 409 | 0.62 | ¹H NMR (600 MHz, CDCl₃) δ 8.51 (d, 1H), 8.39 (d, 1H), 7.98 (dd, 1H), 7.81 (ddd, 1H), 7.68 (dd, 1H), 7.32-7.27 (m, 1H), 7.16 (dd, |
| | | | | | | 1H), 6.84 (t, |
| | | | | | | 1H), 6.61 (dd, |
| | | | | | | 1H), 4.69 (d, |
| | | | | | | 2H), 2.57 (s, |
| | | | | | | 3H). |
| 93 | 1-[4-(difluoro- methyl) benzene-1- sulfonyl]-N- [(5-methyl- pyrazin-2- yl)methyl]- 1H-pyrrole-3- | 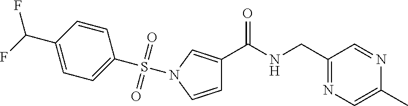 [SMILES: Cc1cnc(CNC(=O)c2ccn(S(=O)(=O)c3ccc(C(F)F)cc3)c2)cn1] | D | 407.1 | 0.58 | ¹H NMR (600 MHz, CDCl₃) δ 8.51 (d, 1H), 8.39 (d, 1H), 7.99 (d, 2H), 7.71 (dd, 1H), 7.68 (d, 2H), 7.17 (dd, 1H), |
| | carboxamide | | | | | 6.84-6.80 (m, |
| | | | | | | 1H), 6.68 (t, |
| | | | | | | 1H), 6.60 (dd, |
| | | | | | | 1H), 4.68 (d, |
| | | | | | | 2H), 2.57 (s, |
| | | | | | | 3H). |
| 94 | 1-(4-methyl- benzene-1- sulfonyl)-N- [(5-methyl- pyrazin-2- yl)methyl]- 1H-pyrrole-3- | 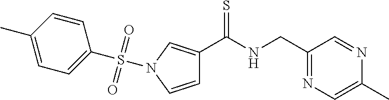 | D | 387.1 | 0.71 | ¹H NMR (600 MHz, CDCl₃) δ 8.70 (t, 1H), 8.55 (d, 1H), 8.40 (d, 1H), 7.83-7.77 (m, 3H), 7.35-7.30 |
| | carbothioamide | | | | | (m, 2H), 7.14 |
| | | | | | | (dd, 1H), 6.68 |
| | | | | | | (dd, 1H), 5.03 |
| | | | | | | (d, 2H), 2.58 |
| | | | | | | (s, 3H), 2.41 |
| | | | | | | (s, 3H). |
| 95 | 1-(2-fluoro- 4-methyl- phenyl) sulfonyl-N- [(5-methyl- pyrazin-2- yl)methyl] pyrrole-3- | 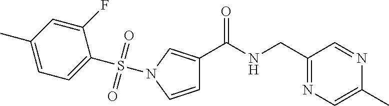 | D | 389.1 | 0.6 | ¹H NMR (500 MHz, DMSO- d₆) δ 8.88 (t, 1H), 8.46 (s, 2H), 7.97-7.86 (m, 2H), 7.39 (d, 1H), 7.35- 7.31 (m, 2H), |
| | carboxamide | | | | | 6.75 (dd, 1H), |
| | | | | | | 4.48 (d, 2H), |
| | | | | | | 2.46 (s, 3H), |
| | | | | | | 2.40 (s, 3H). |
| 96 | 1-(2-fluoro- 4-methoxy- phenyl) sulfonyl-N- [(5-methyl- pyrazin-2- yl)methyl] | 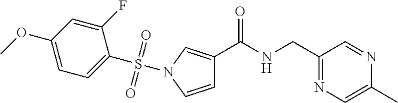 | D | 405.1 | 0.58 | ¹H NMR (600 MHz, DMSO- d₆) δ 8.87 (t, 1H), 8.46 (s, 2H), 7.94 (t, 1H), 7.90-7.87 (m, 1H), 7.34- |
| | pyrrole-3- | | | | | 7.30 (m, 1H), |
| | carboxamide | | | | | 7.16 (dd, 1H), |
| | | | | | | 7.04 (dd, 1H), |
| | | | | | | 6.74 (dd, 1H), |
| | | | | | | 4.48 (d, 2H), |
| | | | | | | 3.87 (s, 3H), |
| | | | | | | 2.46 (s, 3H). |
| 97 | 1-(3-fluoro- 4-methoxy- phenyl) sulfonyl-N- [(5-methyl- pyrazin-2- yl)methyl] pyrrole-3- carboxamide | 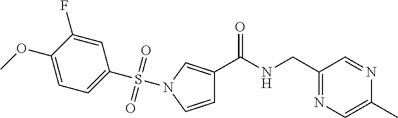 | D | 405.1 | 0.57 | ¹H NMR (600 MHz, DMSO- d₆) δ 8.81 (t, 1H), 8.46 (s, 2H), 7.98 (dd, 1H), 7.92 (dd, 1H), 7.86 (ddd, 1H), 7.46-7.39 (m, 2H), 6.73 |
| | | | | | | (dd, 1H), 4.48 |
| | | | | | | (d, 2H), 3.94 |
| | | | | | | (s, 3H), 2.46 |
| | | | | | | (s, 3H). |
| 98 | 1-(4-methoxy- 2-methyl- phenyl) sulfonyl-N- [(5-methyl- pyrazin-2- yl)methyl] | 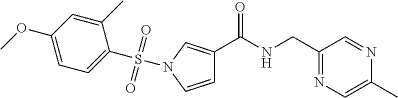 | D | 401.1 | 0.6 | ¹H NMR (600 MHz, DMSO- d₆) δ 8.84 (t, 1H), 8.46 (s, 2H), 7.94 (d, 1H), 7.86 (dd, 1H), 7.33 (dd, |
| | pyrrole-3- | | | | | 1H), 7.08-7.01 |
| | carboxamide | | | | | (m, 2H), 6.71 |
| | | | | | | (dd, 1H), 4.48 |
| | | | | | | (d, 2H), 3.84 |
| | | | | | | (s, 3H), 2.46 |
| | | | | | | (s, 3H), 2.46 |
| | | | | | | (s, 3H). |
| 99 | 1-(4-fluoro- 2,6-dimethyl- phenyl) sulfonyl-N- [(5-methyl- pyrazin-2- yl)methyl] pyrrole-3- | 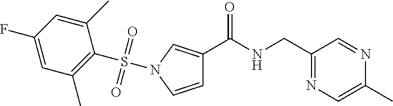 [SMILES: Cc1cnc(CNC(=O)c2ccn(S(=O)(=O)c3c(C)cc(F)cc3C)c2)cn1] | D | 403.2 | 0.65 | ¹H NMR (600 MHz, CDCl₃) δ 8.52 (s, 1H), 8.39 (s, 1H), 7.59 (s, 1H), 7.11 (s, 1H), 6.90 (d, 2H), 6.87 (s, 1H), |
| | carboxamide | | | | | 6.56 (s, 1H), |
| | | | | | | 4.68 (d, 2H), |
| | | | | | | 2.62 (s, 6H), |
| | | | | | | 2.56 (s, 3H). |
| 100 | 1-(4-fluoro- 3,5-dimethyl- phenyl) sulfonyl-N- [(5-methyl- pyrazin-2- yl)methyl] pyrrole-3- | 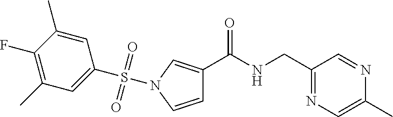 [SMILES: Cc1cnc(CNC(=O)c2ccn(S(=O)(=O)c3cc(C)c(F)c(C)c3)c2)cn1] | D | 403.2 | 0.67 | ¹H NMR (600 MHz, DMSO- d₆) δ 8.82 (t, 1H), 8.45 (s, 2H), 7.90-7.87 (m, 2H), 7.86 (s, 1H), 7.41 (dd, 1H), 6.74 |
| | carboxamide | | | | | (dd, 1H), 4.47 |
| | | | | | | (d, 2H), 2.46 |
| | | | | | | (s, 3H), 2.29 |
| | | | | | | (d, 6H). |
| 101 | 1-(4-fluoro- 3-methyl- phenyl) sulfonyl-N- [(5-methyl- pyrazin-2- yl)methyl] pyrrole-3- | 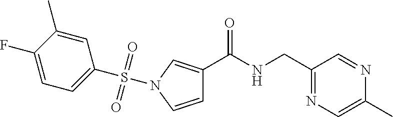 [SMILES: Cc1cnc(CNC(=O)c2ccn(S(=O)(=O)c3ccc(F)c(C)c3)c2)cn1] | D | 389.1 | 0.61 | ¹H NMR (600 MHz, DMSO- d₆) δ 8.82 (t, 1H), 8.45 (s, 2H), 8.06 (ddd, 1H), 7.95-7.91 (m, 1H), 7.90 (dd, 1H), 7.46 |
| | carboxamide | | | | | (t, 1H), 7.42 |
| | | | | | | (dd, 1H), 6.74 |
| | | | | | | (dd, 1H), 4.47 |
| | | | | | | (d, 2H), 2.46 |
| | | | | | | (s, 3H), 2.31 |
| | | | | | | (d, 3H). |
| 102 | 1-(2,3- dihydro- benzofuran- 5-ylsulfonyl)- N-[(5- methyl- pyrazin-2- yl)methyl] | 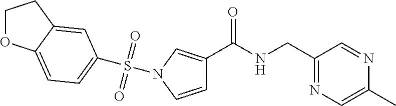 [SMILES: Cc1cnc(CNC(=O)c2ccn(S(=O)(=O)c3ccc4c(c3)CCO4)c2)cn1] | A | 399.1 | 1.93 | 1H NMR (CDCl3 400 MHz): δ 7.72-7.69 (m, 3H), 7.08 (t, J = 2.8 Hz, 1H), 6.82 (d, J = 8.4 Hz, |
| | pyrrole-3- | | | | | 1H), 6.63 (t, |
| | carboxamide | | | | | J = 1.6 Hz, |
| | | | | | | 1H), 4.67 (t, |
| | | | | | | J = 8.8 Hz, |
| | | | | | | 2H), 3.79 (s, |
| | | | | | | 3H), 3.24 (t, |
| | | | | | | J = 8.8 Hz, 2H). |
| 103 | N-[(5- methyl- pyrazin-2- yl)methyl]- 1-(2,4,6- trimethyl- phenyl) sulfonyl- | 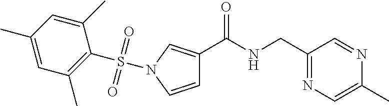 | D | 399.2 | 0.69 | ¹H NMR (500 MHz, DMSO- d₆) δ 8.84 (t, 1H), 8.46 (s, 2H), 7.83 (t, 1H), 7.32 (dd, 1H), 7.18 (s, 2H), 6.70 (dd, |
| | pyrrole-3- | | | | | 1H), 4.48 (d, |
| | carboxamide | | | | | 2H), 2.54 (s, |
| | | | | | | 6H), 2.46 (s, |
| | | | | | | 3H), 2.30 (s, |
| | | | | | | 3H). |
| 104 | 1-(2-chloro- 4-methoxy- phenyl) sulfonyl-N- [(5-methyl- pyrazin-2- yl)methyl] pyrrole-3- | 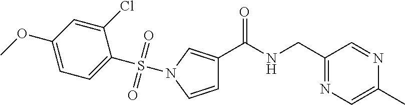 | D | 421.1 | 0.61 | ¹H NMR (500 MHz, DMSO- d₆) δ 8.86 (t, 1H), 8.46 (s, 2H), 8.08 (d, 1H), 7.91 (t, 1H), 7.35 (dd, 1H), 7.32 (d, |
| | carboxamide | | | | | 1H), 7.20 (dd, |
| | | | | | | 1H), 6.73 (dd, |
| | | | | | | 1H), 4.48 (d, |
| | | | | | | 2H), 3.89 (s, |
| | | | | | | 3H), 2.46 (s, |
| | | | | | | 3H). |
| 105 | 1-(2-bromo- 4-methoxy- phenyl) sulfonyl-N- [(5-methyl- pyrazin-2- yl)methyl] pyrrole-3- | 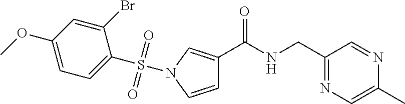 | D | 467 | 0.61 | ¹H NMR (500 MHz, DMSO- d₆) δ 8.85 (t, 1H), 8.46 (s, 2H), 8.06 (d, 1H), 7.92-7.87 (m, 1H), 7.47 (d, 1H), 7.35 (dd, |
| | carboxamide | | | | | 1H), 7.24 (dd, |
| | | | | | | 1H), 6.74 (dd, |
| | | | | | | 1H), 4.48 (d, |
| | | | | | | 2H), 3.88 (s, |
| | | | | | | 3H), 2.46 (s, |
| | | | | | | 3H). |
| 106 | 1-(2-fluoro- 4-methyl- benzene-1- sulfonyl)-N- {[5-(methyl- amino) pyrazin-2- yl]methyl}- 1H-pyrrole-3- | 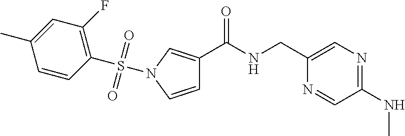 | D | 403.8 | 0.54 | ¹H NMR (CDCl₃ 400 MHz): δ 8.04 (s, 1H), 7.83-7.79 (m, 2H), 7.67 (s, 1H), 7.18 (s, 1H), 7.09 (d, 1H), 6.97 (d, 1H), 6.75 (s, 1H), 6.56-6.55 (m, 1H), 4.73 |
| | carboxamide | | | | | (brs, 1H), 4.52 |
| | | | | | | (d, 2H), 2.96 (s, |
| | | | | | | 3H), 2.40 (s, 3H). |
| 107 | 1-[4-(difluoro- methoxy) benzene-1- sulfonyl]-N- {[5-(methyl- amino) pyrazin-2- yl]methyl}- 1H-pyrrole-3- carboxamide | 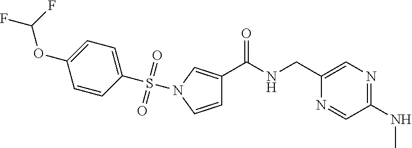 [SMILES: CNc1cnc(CNC(=O)c2ccn(S(=O)(=O)c3ccc(OC(F)F)cc3)c2)cn1] | A | 438.1 | 1.99 | ¹H NMR (CDCl₃ 400 MHz): δ 8.05 (s, 1H), 7.90 (d, 2H), 7.83 (s, 1H), 7.66 (t, 1H), 7.23 (d, 2H), 7.14- 7.13 (m, 1H), 6.64 (m, 1H), 6.58 (t, 1H), 6.56-6.55 (m, |
| | | | | | | 1H), 4.63 (brs, |
| | | | | | | 1H), 4.53 (d, |
| | | | | | | 2H), 2.97 (d, 3H). |
| 108 | 1-(2-fluoro- 4-methyl- benzene-1- sulfonyl)-N- [(2-methyl- pyrimidin-5- yl)methyl]- 1H-pyrrole-3- | 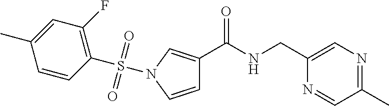 | D | 388.5 | 0.57 | ¹H NMR (CDCl₃ 400 MHz): δ 8.62 (s, 1H), 7.83 (t, 1H), 7.67 (s, 1H), 7.20 (s, 1H), 7.11 (d, 1H), 6.99 (d, 1H), 6.52 (m, 1H), 6.32 (m, |
| | carboxamide | | | | | 1H), 4.53 (d, |
| | | | | | | 2H), 2.71 (s, |
| | | | | | | 3H), 2.42 (s, 3H). |
| 109 | 1-(4-methyl- benzene-1- sulfonyl)-N- [(2-methyl- 2H-1,2,3- triazol-4- yl)methyl]- 1H-pyrrole-3- | 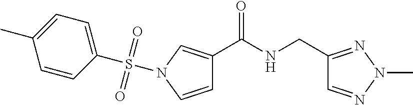 | D | 360.2 | 0.61 | 1H NMR (CDCl3 400 MHz): δ 7.77 (d, J = 8.4 Hz, 2H), 7.68 (m, 1H), 7.52 (s, 1H), 7.32 (d, J = 8.4 Hz, 2H), 7.14 (m, |
| | carboxamide | | | | | 1H), 6.54 (m, |
| | | | | | | 1H), 6.42 (t, J = |
| | | | | | | 4.8 Hz, 1H), 4.60 |
| | | | | | | (d, J = 5.6 Hz, |
| | | | | | | 2H), 4.15 (s, 3H), |
| | | | | | | 2.43 (s, 3H). |
| 110 | 1-(2-fluoro- 4-methyl- benzene-1- sulfonyl)-N- [(2-methoxy- pyrimidin-5- yl)methyl]- 1H-pyrrole-3- carboxamide | 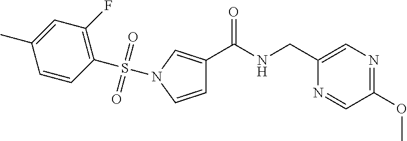 | D | 405.1 | 0.64 | 1H NMR (CDCl3 400 MHz): δ 8.52 (s, 2H), 7.85 (t, J = 8.0 Hz, 1H), 7.67 (m, 1H), 7.21 (s, 1H), 7.12 (d, J = 8.4 Hz, 1H), 7.00 (d, J = 10.8 Hz, 1H), 6.53 (m, |
| | | | | | | 1H), 6.22 (brs, |
| | | | | | | 1H), 4.50 (d, J = |
| | | | | | | 6.0 Hz, 2H), 4.00 |
| | | | | | | (s, 3H), 2.43 (s, |
| | | | | | | 3H). |
| 111 | 1-(benzene- sulfonyl)-N- [(3,5- dimethyl- pyrazin-2- yl)methyl]- 1H-pyrrole-3- carboxamide | 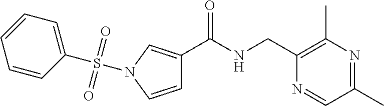 [SMILES: Cc1cnc(CNC(=O)c2ccn(S(=O)(=O)c3ccccc3)c2)c(C)n1] | D | 370.7 | 0.57 | ¹H NMR (CDCl₃ 400 MHz) δ 8.23 (s, 1H), 7.92 (d, 2H), 7.76 (s, 1H), 7.66-7.63 (m, 1H), 7.56-7.52 (m, 2H), 7.46 (brs, 1H), 7.19 |
| | | | | | | (s, 1H), 6.66 (s, |
| | | | | | | 1H), 4.63 (d, |
| | | | | | | 2H), 2.55-2.54 |
| | | | | | | (m, 6H). |
| 112 | 1-[4-(difluoro- methoxy) benzene-1- sulfonyl]-N- [(2-methoxy- pyrimidin-5- yl)methyl]- 1H-pyrrole-3- carboxamide | 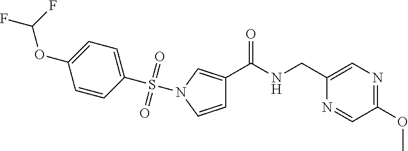 [SMILES: COc1cnc(CNC(=O)c2ccn(S(=O)(=O)c3ccc(OC(F)F)cc3)c2)cn1] | D | 439.0 | 0.6 | 1H NMR (CDCl3 400 MHz): δ 8.49 (s, 2H), 7.90 (d, J = 7.2 Hz, 2H), 7.67 (m, 1H), 7.24 (d, J = 9.2 Hz, 2H), 7.15 (m, 1H), 6.60 (t, J = 72.4 Hz, |
| | | | | | | 1H), 6.54 (m, |
| | | | | | | 1H), 6.36 (t, J = |
| | | | | | | 6.0 Hz, 1H), 4.49 |
| | | | | | | (d, J = 5.6 Hz, |
| | | | | | | 2H), 3.99 (s, 3H). |
| 113 | 1-(benzene- sulfonyl)-N- [(3-chloro- 5-methyl- pyrazin-2- yl)methyl]- 1H-pyrrole-3- carboxamide | 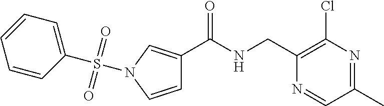 [SMILES: Cc1cnc(CNC(=O)c2ccn(S(=O)(=O)c3ccccc3)c2)c(Cl)n1] | D | 390.7 | 0.65 | 1H NMR (CDCl3 400 MHz): δ 8.32 (s, 1H), 7.91 (d, J = 7.2 Hz, 2H), 7.74 (t, J = 2.0 Hz, 1H), 7.64 (d, J = 7.6 Hz, 1H), |
| | | | | | | 7.54 (t, J = 8.0 |
| | | | | | | Hz, 2H), 7.19- |
| | | | | | | 7.18 (m, 1H), |
| | | | | | | 7.15 (brs, 1H), |
| | | | | | | 6.63 (m, 1H), |
| | | | | | | 4.76 (d, J = 4.4 |
| | | | | | | Hz, 2H), 2.57 |
| | | | | | | (s, 3H). |
| 114 | 1-(4-methyl- benzene-1- sulfonyl)-N- [(2-methyl- 1,3-thiazol- 5-yl)methyl]- 1H-pyrrole-3- carboxamide | 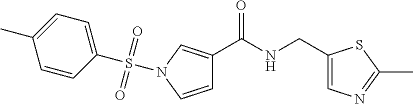 | D | 376.2 | 0.58 | ¹H NMR (CDCl₃ 400 MHz): δ 7.76 (d, 2H), 7.65 (s, 1H), 7.47 (s, 1H), 7.31 (d, 2H), 7.13 (s, 1H), 6.49 (s, 1H), 6.18 (m, 1H), 4.66 (d, 2H), |
| | | | | | | 2.65 (s, 3H), 2.41 |
| | | | | | | (s, 3H). |
| 115 | 1-(4-methyl- benzene-1- sulfonyl)-N- [(5-methyl- 1,3,4- thiadiazol-2- yl)methyl]- 1H-pyrrole-3- | 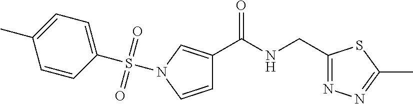 | D | 377.2 | 0.61 | ¹H NMR (CDCl₃ 400 MHz): δ 7.77 (d, 2H), 7.74 (s, 1H), 7.31 (d, 2H), 7.14 (t, 1H), 6.90 (brs, 1H), 6.56 (s, 1H), 4.89 (d, 2H), 2.74 (s, |
| | carboxamide | | | | | 3H), 2.41 (s, 3H). |
| 116 | 1-(4-methyl- benzene-1- sulfonyl)-N- [(3-methyl- 1H-pyrazol- 5-yl)methyl]- 1H-pyrrole-3- carboxamide | 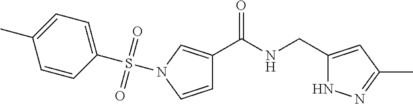 | D | 359.2 | 0.56 | ¹H NMR (DMSO-d₆ 400 MHz): δ 12.21 (s, 1H), 8.51 (s, 1H), 7.87 (m, 3H), 7.46 (d, J = 7.2 Hz, 2H), 7.36 (s, 1H), 6.70 |
| | | | | | | (s, 1H), 5.85 (s, |
| | | | | | | 1H), 4.26 (s, 2H), |
| | | | | | | 2.38 (s, 3H), 2.15 |
| | | | | | | (s, 3H). |
| 117 | 1-(2-chloro- 4-methoxy- benzene-1- sulfonyl)-N- [(1-methyl- 1H-pyrazol- 3-yl)methyl]- 1H-pyrrole-3- carboxamide | 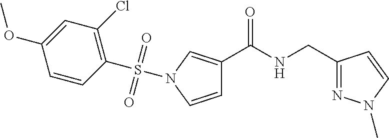 [SMILES: COc1ccc(S(=O)(=O)n2ccc(C(=O)NCc3ccn(C)n3)c2)c(Cl)c1] | D | 409.2 | 0.63 | ¹H NMR (CDCl₃ 400 MHz): δ 8.03 (d, 1H), 7.65 (s, 1H), 7.27 (m, 1H), 7.16 (t, 1H), 6.96 (d, 1H), 6.91-6.88 (m, 1H), 6.53-6.50 (m, 2H), 6.17 |
| | | | | | | (d, 1H), 4.53 (d, |
| | | | | | | 2H), 3.86 (s, |
| | | | | | | 3H), 2.84 (s, 3H). |
| 118 | 1-(2-chloro- 4-methoxy- benzene-1- sulfonyl)-N- [(5-methyl- pyrimidin-2- yl)methyl]- 1H-pyrrole-3- | 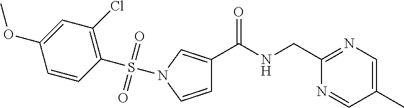 | D | 420.8 | 0.62 | ¹H NMR (DMSO-d₆ 400 MHz): δ 8.78 (t, 1H), 8.59 (s, 2H), 8.08 (d, 1H), 7.89 (d, 1H), 7.35 (d, 1H), |
| | carboxamide | | | | | 7.33 (d, 1H), |
| | | | | | | 7.21 (d, 1H), |
| | | | | | | 6.74-6.72 (m, |
| | | | | | | 1H), 4.53 (d, |
| | | | | | | 2H), 3.89 (s, |
| | | | | | | 3H), 2.24 (s, |
| | | | | | | 3H). |

### Biological Evaluation:

### Cell Culture

HEK-293 cells stably expressing hKv3.1b were used for the experiments. Cells were cultured in DMEM medium supplemented with 10% Fetal Bovine Serum, 100 ug/mL Geneticidin and 100 u/mL Penicillin/Streptomycin (all from Gibco). Cells were grown to 80-90% confluency at 37° C. and 5% CO₂. On the day of the experiment the cells were detached from the tissue culture flasks by Detachin, resuspended in serum free medium containing 25 mM HEPES and transferred to the cell hotel of the QPatch. The cells were used for experiments 0-5 hours after detachment.

### Electrophysiology

Patch-clamp recordings were performed using the automated recording system QPatch-16x (Sophion Bioscience, Denmark). Cells were centrifuged, SFM removed and the cells were resuspended in extracellular buffer containing (in mM): 145 NaCl, 4 KCl, 1 MgCl₂, 2 CaCl₂, 10 HEPES and 10 glucose (added fresh on the day of experiment); pH 7.4 adjusted with NaOH, 305 mOsm adjusted with sucrose.

Single cell whole-cell recordings were carried out using an intracellular solution containing (in mM): 120 KCl, 32.25/10 KOH/EGTA, 5.374 CaCl₂, 1.75 MgCl₂, 10 HEPES, 4 Na₂ATP (added fresh on the day), pH 7.2 adjusted with KOH, 395 mOsm adjusted with sucrose. Cell membrane potentials were held at −80 mV and currents were evoked by voltage steps (200 ms duration) from −70 mV to +10 mV (in 10 mV increments). Vehicle (0.33% DMSO) or increasing concentration of compound (I) were applied and the voltage protocol was run 3 times (resulting in 3 min cpd incubation time). Five increasing concentrations of compound (I) were applied to each cell.

Leak subtraction protocol was applied at −33% of the sweep amplitude, and serial resistance values were constantly monitored.

Any cell where serial resistance exceeded 25 MOhm, membrane resistance less than 200 MOhm or current size at −10 mV less than 200 pA was eliminated from the subsequent analysis.

### Data Analysis

Data analysis was performed using Sophion's QPatch assay software in combination with Microsoft Excel™ (Redmond, Wash., USA). Current voltage relationships were plotted from the peak current at the individual voltage steps normalized to the vehicle addition at 10 mV. The voltage threshold for channel activation was defined as 5% activation of the peak current at 10 mV in presence of vehicle. The activity of the compounds was described as the ability to shift this current voltage relationship to more hyperpolarized potentials and is given as the maximum absolute shift possible at the tested concentrations (0.37, 1.11, 3.33, 10, 30 μM). Concentration response curves were plotted from the threshold shift at the individual concentrations and were fitted excel fit model 205 sigmoidal dose-response model (fit=A+((B−A)/1+((C/x)\hat{ }D)))), where A is the minimum value, B the maximum value, C the EC₅₀ value and D the slope of the curve. The concentration needed to shift the threshold 5 mV was readout from this curve (ECdelta5 mV).

### Compound Effects

In the assay described above, the compounds of the invention had the following biological activity:

| | Threshold Shift | ECdelta5 mV |
|---|---|---|
| Compound | (mV) | (nM) |
| 1 | 27 | 1600 |
| 2 | 40 | 750 |
| 3 | 25 | 1100 |
| 4 | 17 | 3100 |
| 5 | 10 | 7800 |
| 6 | 24 | 2300 |
| 7 | 33 | 1300 |
| 8 | 12 | 4000 |
| 9 | 15 | 2400 |
| 10 | 10 | 3600 |
| 11 | 16 | 1300 |
| 12 | 16 | 1700 |
| 13 | 13 | 1700 |
| 14 | 23 | 1500 |
| 15 | 14 | 1700 |
| 16 | 16 | 3000 |
| 17 | 16 | 2300 |
| 18 | 10 | 3500 |
| 19 | 11 | 2100 |
| 24 | 24 | 3200 |
| 25 | 37 | 1100 |
| 26 | 22 | 930 |
| 27 | 19 | 1500 |
| 28 | 27 | 1500 |
| 29 | 31 | 1300 |
| 30 | 17 | 2500 |
| 31 | 13 | 9600 |
| 32 | 16 | 1300 |
| 33 | 15 | 2200 |
| 34 | 18 | 1100 |
| 35 | 21 | 1100 |
| 36 | 27 | 810 |
| 37 | 22 | 1800 |
| 38 | 13 | 4600 |
| 39 | 17 | 1600 |
| 40 | 22 | 2000 |
| 41 | 17 | 3100 |
| 42 | 25 | 1700 |
| 43 | 23 | 1700 |
| 47 | 12 | 2300 |
| 48 | 18 | 2300 |
| 49 | 13 | 2100 |
| 50 | 13 | 3300 |
| 51 | 14 | 2400 |
| 52 | 13 | 2500 |
| 53 | 15 | 2300 |
| 54 | 15 | 2000 |
| 55 | 15 | 1500 |
| 56 | 14 | 6800 |
| 57 | 14 | 3600 |
| 58 | 22 | 2200 |
| 59 | 14 | 2000 |
| 60 | 16 | 2200 |
| 61 | 12 | 5900 |
| 62 | 12 | 4900 |
| 63 | 13 | 2200 |
| 64 | 13 | 4400 |
| 65 | 14 | 4200 |
| 66 | 18 | 1400 |
| 67 | 23 | 2200 |
| 68 | 21 | 2300 |
| 69 | 18 | 3000 |
| 70 | 17 | 1100 |
| 71 | 19 | 1800 |
| 72 | 20 | 2800 |
| 73 | 13 | 2100 |
| 74 | 21 | 2100 |
| 75 | 14 | 3200 |
| 76 | 17 | 3600 |
| 77 | 12 | 3300 |
| 78 | 17 | 1900 |
| 80 | 12 | 2800 |
| 81 | 28 | 1000 |
| 82 | 14 | 4100 |
| 83 | 13 | 5000 |
| 84 | 12 | 4600 |
| 85 | 18 | 3100 |
| 86 | 17 | 2800 |
| 87 | 15 | 4000 |
| 88 | 23 | 2000 |
| 89 | 23 | 660 |
| 90 | 32 | 1300 |
| 91 | 20 | 1800 |
| 92 | 12 | 3400 |
| 93 | 34 | 1500 |
| 94 | 19 | 1100 |
| 95 | 24 | 1100 |
| 96 | 35 | 1300 |
| 97 | 18 | 3200 |
| 98 | 36 | 710 |
| 99 | 21 | 1900 |
| 100 | 15 | 4300 |
| 101 | 23 | 2000 |
| 102 | 32 | 1400 |
| 103 | 24 | 720 |
| 104 | 31 | 1000 |
| 105 | 24 | 1200 |
| 106 | 42 | 550 |
| 107 | 36 | 1800 |
| 108 | 35 | 1300 |
| 109 | 29 | 1200 |
| 110 | 24 | 1300 |
| 111 | 21 | 2900 |
| 112 | 17 | 4100 |
| 113 | 14 | 3500 |
| 114 | 15 | 1600 |
| 115 | 17 | 2800 |
| 116 | 25 | 2600 |
| 117 | 21 | 1500 |
| 118 | 20 | 2500 |

Manual Patch Clamp Electrophysiological Evaluation, hKv3.1, hKv3.2, hKv3.3, hKv3.4:

### Cell Cultures

HEK-293 cells stably expressing human Kv3.1b, Kv3.2, Kv3.3 or Kv3.4 were used for the experiments.

Kv3.1b, Kv3.2: Cells were cultured in MEM medium supplemented with 10% Fetal Bovine Serum, 1% Penicillin/Streptomycin, 2 mM glutamine and 0.6 mg/mL geneticin. Cells were grown to 80-90% confluency at 37° C. and 5% CO₂
Kv3.3 or Kv3.4: Cells were cultured in DMEM medium supplemented with 10% Fetal Bovine Serum, 500 ug/mL Geneticidin and 1% Penicillin/Streptomycin. Cells were grown to 80-90% confluency at 37° C. and 5% CO₂.

On the day of the experiment the cells were detached by TrypLE and resuspended in culture medium. Cells were centrifuged, media removed and the cells were resuspended in extracellular buffer containing (in mM): 130 Na-gluconate, 20 NaCl, 4 KCl, 1 MgCl₂, 1.8 CaCl₂, 10 HEPES and 5 glucose, pH 7.3 adjusted with NaOH, 310-320 mOsm

### Electrophysiology

Patch-clamp recordings were performed using a manual patch-clamp system (Axon Multiclamp 700B, Digidata 1440, pCLAMP 10, Molecular Devices Corporation) with a fast perfusion system (RSC-160 Rapid solution Changer, BioLogic). Whole-cell recordings were carried out using an intracellular solution containing (in mM): 100 K-gluconate, 40 KCl, 10 HEPES, 1 EGTA, 1 MgCl₂, pH 7.2 adjusted with KOH, 290-300 mOsm. Cell membrane potentials were held at −80 mV and current voltage-relationship was generated by voltage steps (50 ms duration) from −100 mV to +10 mV (in 10 mV increments) and then back to −100 mV for 50 ms, with inter-sweep interval of 3 s. The peak current amplitude of −10 mV was monitored until stable (<5% change) by using one step voltage protocol. One IV protocol was run as baseline, then compound perfusion was stared and peak current stability was monitored with single step protocol prior to the IV protocol. Single concentrations were measured per cell. Acceptable cells had seal resistance >500 MOhm, Access resistance <10 MOhm, and leak current <200 pA.

### Data Analysis:

Data analysis was performed using Clampfit (V10.2) in combination with Microsoft Excel™ (Redmond, Wash., USA). Current voltage relationships were plotted from the peak current (baseline subtracted) at the individual voltage steps normalized to the vehicle addition at 10 mV. The voltage threshold for channel activation was defined as 5% activation of the peak current at 10 mV in presence of vehicle. The activity of the compounds was described as the ability to shift this current voltage relationship to more hyperpolarized potentials and is given as the maximum absolute shift possible at the tested concentrations (0.37, 1.11, 3.33, 10, 30 μM). Concentration response curves were plotted from the threshold shift at the individual concentrations and were fitted excel fit model 205 sigmoidal dose-response model (fit=A+((B−A)/1+((C/x)\hat{ }D)))), where A is the minimum value, B the maximum value, C the EC₅₀ value and D the slope of the curve. The concentration needed to shift the threshold 5 mV was readout from this curve (EC_{Δ5 mV}), as well as the ability to increase the peak current at the −10 mV step (EC_{30% increase}). Concentrations that inhibited the current, rather than potentiating, were excluded from the data analysis.

It was a general observation that the highest concentration (30 μM) would inhibit the current rather than potentiating it, resulting in a bell-shaped concentration response curve. For the curve fitting, only the potentiating datapoints were included.

### Compound Effects:

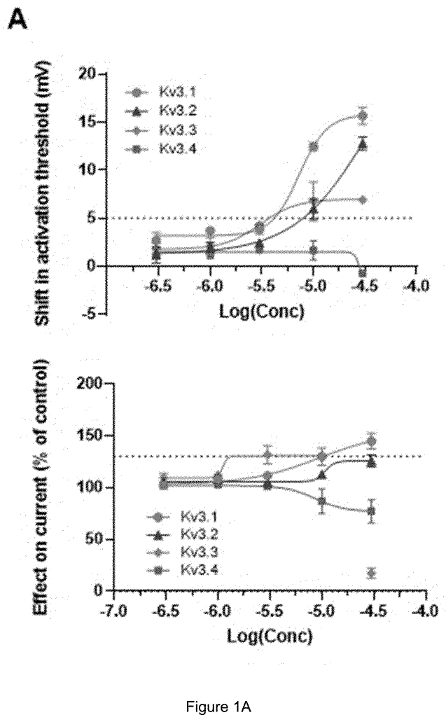

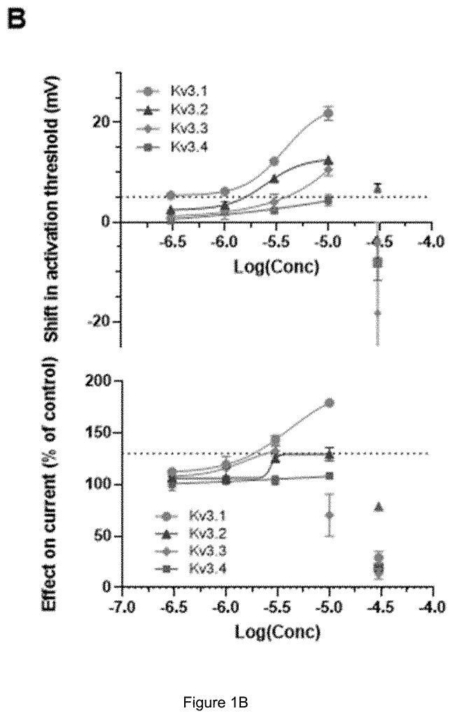

The effects of selected compound examples (Compound 86 and Compound 90) are illustrated in FIG. 1 and Table 2.

TABLE 2
Potencies on Kv3.x measured by manual patch clamp electrophysiology. Potencies are
given as the effective concentration that can shift the activation threshold by 5 mV in the
hyperpolarized direction, or as the concentration needed for increasing the current by 30% at
the −10 mV depolarizing step. All concentrations are given in μM. For Kv3.1, the potencies
measured by automated patch clamp electrophysiology (Qpatch) are provided for reference.

| | hKv3.1 (Qpatch) | | hKv3.1 | | hKv3.2 | | hKv3.3 | | hKv3.4 | |
|---|---|---|---|---|---|---|---|---|---|---|
| | | ECΔ30% | | ECΔ30% | | ECΔ30% | | ECΔ30% | | ECΔ30% |
| Compound | ECΔ5 mV | increase | ECΔ5 mV | increase | ECΔ5 mV | increase | ECΔ5 mV | increase | ECΔ5 mV | increase |
| 86 | 2.7 | 2.9 | 3.0 | 10.1 | 6.0 | >30 | 4.9 | >30 | 14.4 | >30 |
| 90 | 1.2 | 1.6 | 0.6 | 1.8 | 1.5 | 8.2 | 2.7 | 2.7 | >30 | >30 |

### Off-Target Profile on Key Ion Channels Targets:

The activity of selected compound examples at three key ion channel off targets was measured, namely Nav1.1, Kv1.1/1.2 and Kv7.2/7.3.

The voltage gated sodium channel, Nav1.1, is known to have state-dependent pharmacology, therefore, compound examples were tested for effects on inhibition or activation at the resting state channel, a use-dependent readout, and an inactivated state readout by electrophysiology, at concentrations up to 30 μM.

Effects of selected examples on inhibition of the voltage gated heteromeric potassium channel Kv1.1/1.2 was also tested in a use-dependent manner by electrophysiology at concentrations up to 30 μM.

Effects of selected examples on activation of the voltage gated heteromeric potassium channel Kv7.2/7.3 was tested in a fluorescence-based ion flux assay at concentrations up to 30 μM.

The results are summarized in Table 3

TABLE 3
Summary of effects at key ion channel off targets

| | hNav1.1 (Qpatch) | hKv1.1/1.2 (Qpatch) | | |
|---|---|---|---|---|
| Com- | Multiple state | | Use- | hKv7.2/7.3 |
| pound | readouts | Resting | dependent | (FDSS) |
| 86 | EC₅₀/IC₅₀ > 30 uM | IC₅₀ > 30 uM | IC₅₀ = 30 uM | EC₅₀ > 30 uM |
| 90 | EC₅₀/IC₅₀ > 30 uM | IC₅₀ > 30 uM | IC₅₀ > 30 uM | EC₅₀ > 30 uM |

### Ex-Vivo Evaluation

### Animals

Male Sprague Dawley rats (18˜24 days old) from Shanghai Laboratory Animal Center (Shanghai, China) were used for brain slice experiments. They were housed in groups of five in controlled conditions (temperature of 23±3° C., humidity of 40-70%, and 12:12 light-dark cycle with lights on at 5:00 am) and free access to food and water. All procedures were conducted in agreement with the guideline of Institutional Animal Care and Use Committee at ChemPartner. Ethical approval was obtained by the The Danish Animal Experimentation Inspectorate (journal no. 2014 15 0201 00339).

### Hippocampal Brain Slice Preparation

Animals were decapitated by a guillotine and their brains quickly removed and placed in ice-cold modified artificial cerebral spinal fluid (ACSF) containing (in mM): 110 sucrose, 60 NaCl, 3 KCl, 5 glucose, 28 NaHCO₃, 1.25 NaH₂PO₄, 0.5 CaCl₂ and 7 MgCl₂, aerated with 95% O₂/5% CO₂. The brains were block-trimmed and glued onto the stage of a vibratome (VT1200S, Leica Microsystems Inc., Bannockburn, Ill., USA). Parasagittal hippocampal slices (300 μm) were cut and incubated in the regular carbogenated ACSF containing (in mM): 119 NaCl, 2.5 KCl, 1.2 Na₂HPO₄, 25 NaHCO₃, 2.5 CaCl₂, 1.3 MgCl₂, 10 glucose at 35° C. for the first 60 min and then transferred to room temperature prior to recordings.

### Electrophysiological Brain Slice Recordings

In the hippocampal CA1 pyramidal cell layer, fast-spiking interneurons (FSI) or pyramidal (PYR) cells were visualized using differential interference contrast-infrared (DIC-IR)-assisted microscopy and whole-cell patch clamp recordings performed using an Axon Multiclamp 700B amplifier (Molecular Devices, Union City, Calif.). FSI were selected based on non-pyramidal shape and multipolar dendrites. Putative FSI were only accepted for experiments if they fulfilled the following electrophysiological criteria: short duration action potentials (APs <1 ms), large after hyperpolarizations, and, in response to sustained current injection, high frequency AP firing (>100 Hz) with limited spike frequency adaptation. Patch pipettes (4-5MΩ) were pulled from thick-walled borosilicate glass tubing (O.D.: 1.5 mm, I.D.: 0.75 mm; Sutter Instrument, Novato, Calif., USA).

Whole cell patch clamp recordings in current clamp mode were used to study neuronal excitability. AP firing was recorded in the presence of 50 μM APV, 10 μM DNQX and 10 μM Gabazine to block all synaptic transmission mediated by NMDA, AMPA and GABA_{A} receptors. Patch pipettes were filled with an intracellular solution containing (in mM): 110 KMeSO₄, 10 HEPES, 1 EGTA, 2 MgCl₂, 4 Na₂-ATP, 0.4 TRIS-GTP, 10 Tris₂-Phosphocreatine, pH adjusted to 7.3 with KOH. The osmolarity was adjusted to 290 mOsm with sucrose. The holding potential was maintained continuously at −70 mV by manual DC injection. Series resistance (10-20 MΩ after “break-in”) was 90% compensated and monitored constantly during the entire experiment by “bridge”-balancing of the instantaneous voltage responses to a hyperpolarizing current pulse before each depolarizing stimulus delivery. A series of depolarizing current steps (800 ms-long) were applied every 3 min. Following at least 15 min of stable activity, Kv3 channel modulators were applied to the ACSF at increasing concentrations.

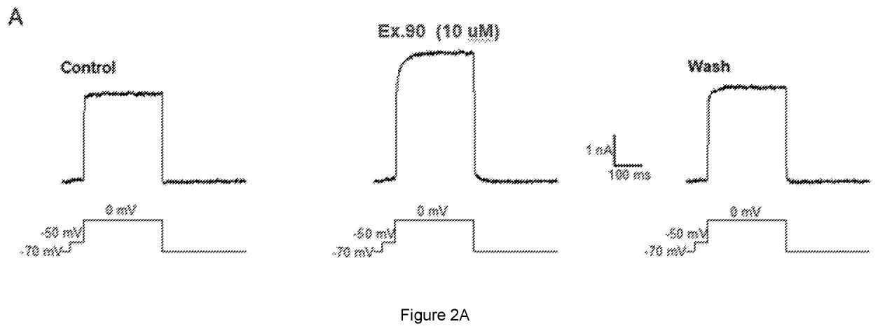

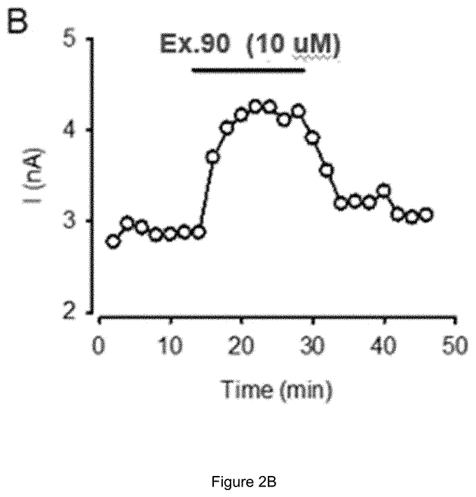

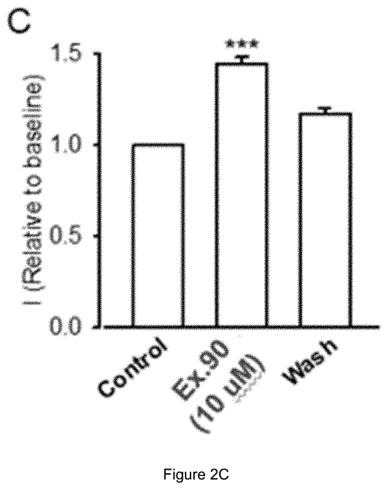

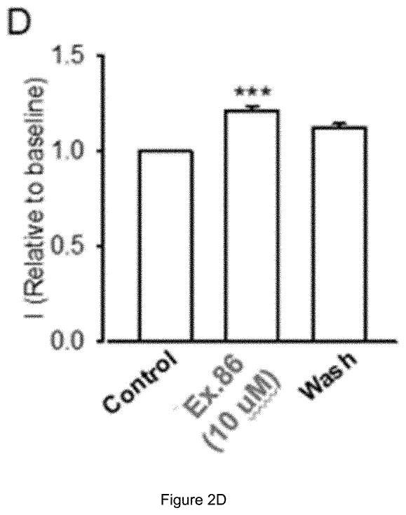

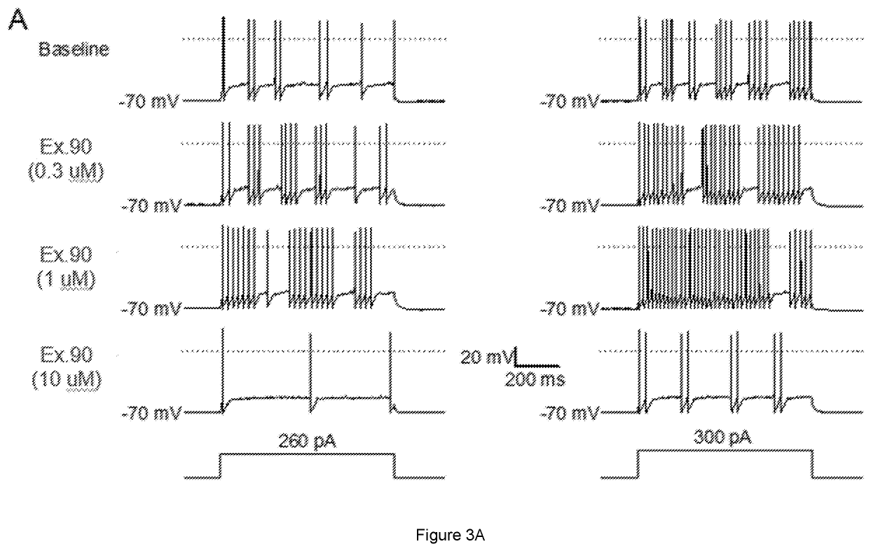

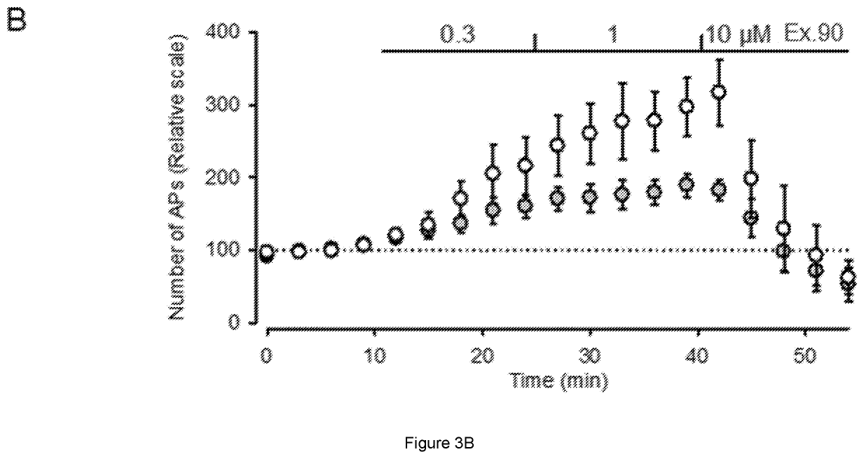

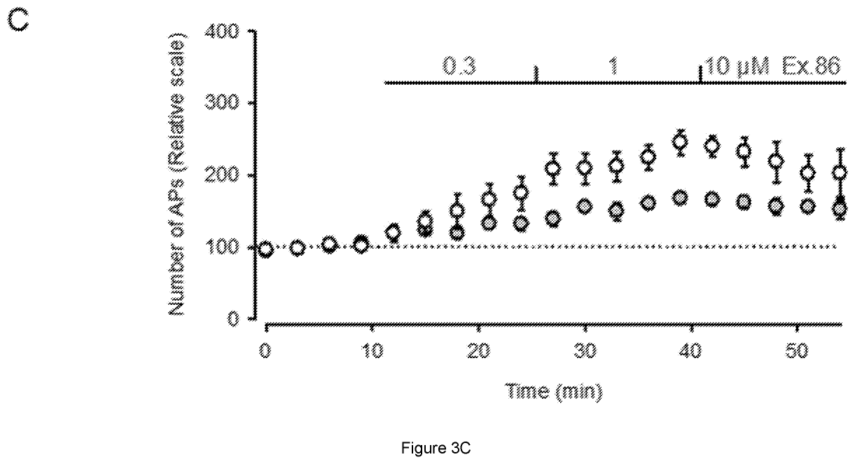

Whole cell patch clamp recordings in voltage clamp mode were used to study the outward K⁺ current from FSI or PYR cells. The intracellular solution contained (in mM): 130 K-gluconate, 10 HEPES, 10 BAPTA, 1 MgCl₂, 0.2 Na₂-ATP, 0.3 TRIS-GTP, 4 Tris₂-Phosphocreatine, pH adjusted to 7.3 with KOH. The osmolarity was adjusted to 295 mOsm with sucrose. Outward K⁺ current was recorded in the presence of 1 μM TTX and 10 μM DNQX in the ACSF to inhibit voltage-gated Na⁺ channels and AMPA channels, respectively. Cells were voltage clamped at −70 mV. To inactivate transient currents a 50 ms pulse to −50 mV was applied before outward current was activated by a 300 ms step to 0 mV. The protocol was repeated every 2 min. Following stable baseline recordings, Kv3 channel modulators were applied to the ACSF. For all recordings, the access resistance was monitored throughout the experiments. Neurons whose series resistance changed by >15% were excluded from the analyses. Experimental temperature was 26-27° C. Results are illustrated in FIG. 2 and FIG. 3.

### In Vivo Pharmacokinetic Time Profile:

### Animals

Male Sprague Dawley rats or male C57 mice from SLAC Laboratory Animal Co. Ltd., Shanghai, China or SIPPR/BK Laboratory Animal Co. Ltd., Shanghai, China were used for pharmacokinetic studies. Animals were group housed during acclimation and individually housed during in-life. The animal room environment was controlled (conditions: temperature 20 to 26° C., relative humidity 30 to 70%, 12 hours artificial light and 12 hours dark) and all animals have access to Certified Rodent Diet (Beijing KEAO XIELI Feed Co., Ltd. Beijing, P.R. China.) ad libitum. Animals were deprived of food overnight prior to dosing and fed approximately 4 hours post-dosing. Water was autoclaved before provided to the animals ad libitum.

For oral dosing, the dose formulation was administered via oral gavage.

### Blood Sample Collection and Processing:

Animals were anesthetized via isoflurane. At terminal time point, about 200 μL blood was collected from cardiac puncture or abdominal vein. All blood samples were transferred into microcentrifuge tubes containing 5 μL of K₂EDTA (0.5 M) as anti-coagulant and placed on wet ice until processed for plasma by centrifugation (3,000 rpm for 5 minutes at 2 to 8° C.) within half an hour of collection and kept at −70±10° C. until LC/MSMS analysis

### Brain Sample Collection and Processing:

After blood collection, brain was harvested and washed twice with cold deionized water, and blotted on filter paper, weighted and frozen until processed. Brain samples were thawed and homogenized with 4-fold of cold water using Covaris (peak power 450.0, Duty Factor 20.0, Cycles/Burst 200). for 3 min, vortex for 10 second every 1 min. Samples were further stored at −79° C. (dilution factor=5) until bioanalysis

### Results:

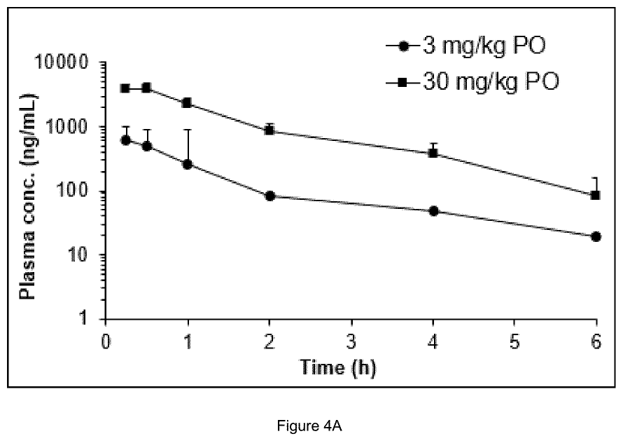

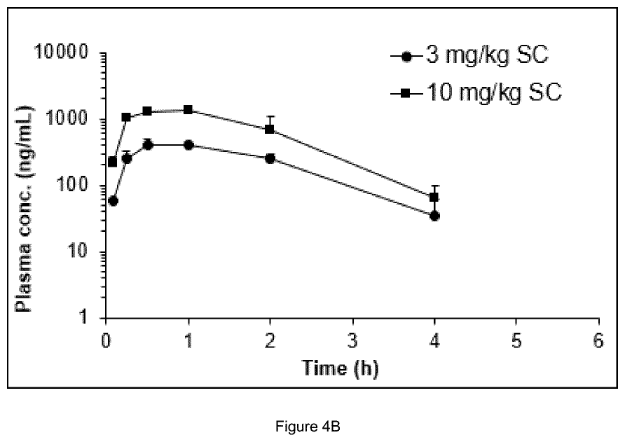

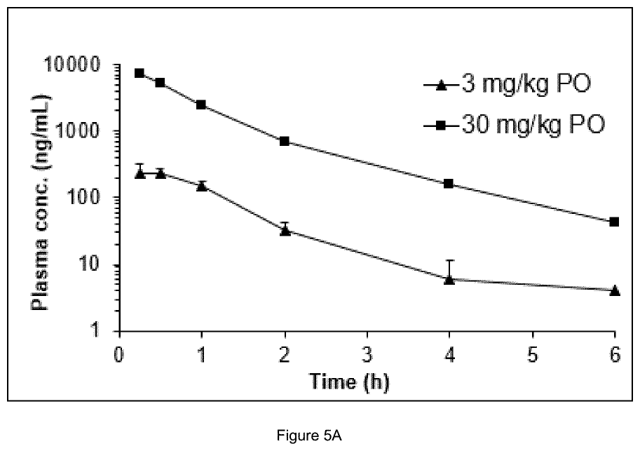

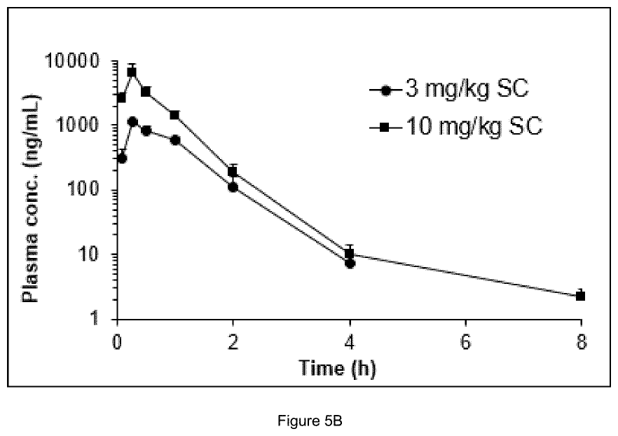

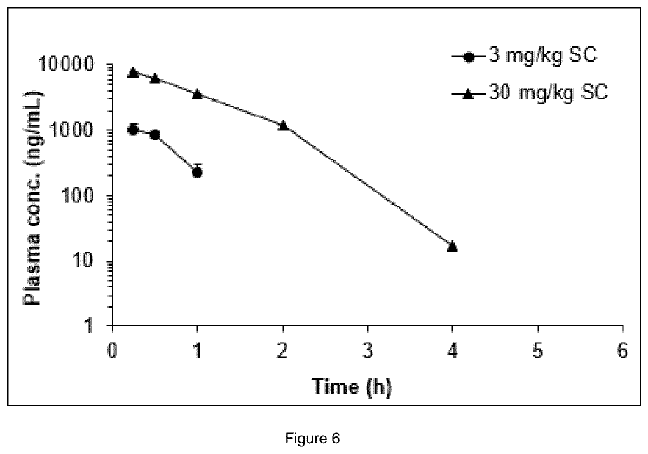

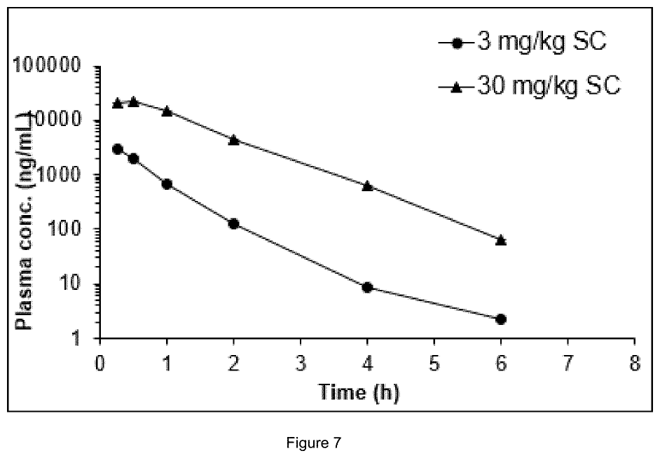

The in vivo pharmacokinetic time profile of selected compound examples (Compound 86 and Compound 90) in rats and mice are illustrated in FIGS. 4-7 and summarized in Tables 4-7.

### Compound 90

TABLE 4
Rat: (Vehicle = 10% HP-betaCD)

| | PO administration | | SC administration | |
|---|---|---|---|---|
| Dose (mg/mg) | 3 | 30 | 3 | 10 |
| Cmax, plasma (ng/mL) | 627 | 3940 | 427 | 1373 |
| T½ (h) | 2.0 | 1.1 | 0.8 | 0.7 |
| Estimated* unbound | 65 | 406 | 44 | 142 |
| brain Cmax (nM) | | | | |
| Unbound** plasma, | 160 | 1015 | 110 | 353 |
| Cmax (nM) | | | | |

*based on measured brain/plasma ratio of 0.5 and unbound fraction in brain = 8%.
**unbound fraction in plasma = 10%.

TABLE 5
Mouse: Vehicle = 10% HP-betaCD

| | PO administration | | SC administration | |
|---|---|---|---|---|
| Dose (mg/mg) | 3 | 30 | 3 | 10 |
| Cmax, plasma (ng/mL) | 274 | 7353 | 1130 | 6360 |
| T½ (h) | 1.0 | 1.0 | 0.5 | 1.3 |
| Estimated* unbound | 28 | 753 | 116 | 656 |
| brain Cmax (nM) | | | | |
| Unbound** plasma, Cmax (nM) | 70 | 1895 | 291 | 1639 |

*based on measured brain/plasma ratio of 0.4 and unbound fraction in brain = 10%
**unbound fraction in plasma = 10%

### Compound 86

TABLE 6
Rat: (Vehicle = 10% HP-betaCD)

| | SC administration | |
|---|---|---|
| Dose (mg/mg) | 3 | 30 |
| Cmax, plasma (ng/mL) | 1095 | 7620 |
| T½ (h) | 0.3 | 0.4 |
| Estimated* unbound brain Cmax (nM) | 95 | 663 |
| Unbound** plasma, Cmax (nM) | 380 | 2650 |

*based on measured brain/plasma ratio of 0.25 and unbound fraction in brain = 12%.
**unbound fraction in plasma = 12%.

TABLE 7
Mouse: Vehicle = 10% HP-betaCD

| | SC administration | |
|---|---|---|
| Dose (mg/mg) | 3 | 30 |
| Cmax, plasma (ng/mL) | 3020 | 21970 |
| T½ (h) | 0.7 | 0.6 |
| Estimated* unbound brain Cmax (nM) | 289 | 2100 |
| Unbound** plasma, Cmax (nM) | 700 | 5094 |

*based on measured brain/plasma ratio of 0.3 and unbound fraction in brain = 11%
**unbound fraction in plasma = 8%

### REFERENCES

Bartos M, Vida I, Jonas P. Synaptic mechanisms of synchronized gamma oscillations in inhibitory interneuron networks. Nat Rev Neurosci. 2007 January; 8(1):45-56. Review.
Chien L Y, Cheng J K, Chu D, Cheng C F, Tsaur M L. Reduced expression of A-type potassium channels in primary sensory neurons induces mechanical hypersensitivity. J Neurosci. 2007 Sep. 12; 27(37):9855-65. PubMed PMID: 17855600.
Chow A, Erisir A, Farb C, Nadal M S, Ozaita A, Lau D, Welker E, Rudy B. K(+) channel expression distinguishes subpopulations of parvalbumin- and somatostatin-containing neocortical interneurons. J Neurosci. 1999 Nov. 1; 19(21):9332-45.
Edden R A, Crocetti D, Zhu H, Gilbert D L, Mostofsky S H. Reduced GABA concentration in attention-deficit/hyperactivity disorder. Arch Gen Psychiatry. 2012 July; 69(7):750-3. doi: 10.1001/archgenpsychiatry.2011.2280.
Foss-Feig J H, Adkinson B D, Ji J L, Yang G, Srihari V H, McPartland J C, Krystal J H, Murray J D, Anticevic A. Searching for Cross-Diagnostic Convergence: Neural Mechanisms Governing Excitation and Inhibition Balance in Schizophrenia and Autism Spectrum Disorders. Biol Psychiatry. 2017 May 15; 81(10):848-861. doi: 10.1016/j.biopsych.2017.03.005. Epub 2017 Mar. 14. Review.
Fuchs T, Jefferson S J, Hooper A, Yee P H, Maguire J, Luscher B. Disinhibition of somatostatin-positive GABAergic interneurons results in an anxiolytic and antidepressant-like brain state. Mol Psychiatry. 2017 June; 22(6):920-930. doi: 10.1038/mp.2016.188. Epub 2016 Nov. 8.
Herrmann C S, Demiralp T. Human EEG gamma oscillations in neuropsychiatric disorders. Clin Neurophysiol. 2005 December; 116(12):2719-33. Epub 2005 Oct. 25. Review.
Kaczmarek L K, Zhang Y. Kv3 Channels: Enablers of Rapid Firing, Neurotransmitter Release, and Neuronal Endurance. Physiol Rev. 2017 Oct. 1; 97(4):1431-1468. doi: 10.1152/physrev.00002.2017. Review.
Klempan T A, Sequeira A, Canetti L, Lalovic A, Ernst C, ffrench-Mullen J, Turecki G. Altered expression of genes involved in ATP biosynthesis and GABAergic neurotransmission in the ventral prefrontal cortex of suicides with and without major depression. Mol Psychiatry. 2009 February; 14(2):175-89. Epub 2007 Oct. 16
Kudo T, Loh D H, Kuljis D, Constance C, Colwell C S. Fast delayed rectifier potassium current: critical for input and output of the circadian system. J Neurosci. 2011 Feb. 23; 31(8):2746-55. doi: 10.1523/JNEUROSCI.5792-10.2011.
Lau D, Vega-Saenz de Miera E C, Contreras D, Ozaita A, Harvey M, Chow A, Noebels J L, Paylor R, Morgan J I, Leonard C S, Rudy B. Impaired fast-spiking, suppressed cortical inhibition, and increased susceptibility to seizures in mice lacking Kv3.2 K+ channel proteins. J Neurosci. 2000 Dec. 15; 20(24):9071-85.
Lin L C, Sibille E. Reduced brain somatostatin in mood disorders: a common pathophysiological substrate and drug target? Front Pharmacol. 2013 Sep. 9; 4:110. doi: 10.3389/fphar.2013.00110. Review.
Macica C M, von Hehn C A, Wang L Y, Ho C S, Yokoyama S, Joho R H, Kaczmarek L K. Modulation of the kv3.1b potassium channel isoform adjusts the fidelity of the firing pattern of auditory neurons. J Neurosci. 2003 Feb. 15; 23(4):1133-41.
Muona M, et al. A recurrent de novo mutation in KCNC1 causes progressive myoclonus epilepsy. Nat Genet. 2015 January; 47(1):39-46.
Oliver K L, et al. Myoclonus epilepsy and ataxia due to KCNC1 mutation:Analysis of 20 cases and K(+) channel properties. Ann Neurol. 2017 May; 81(5):677-689. doi: 10.1002/ana.24929
Palop J J, Mucke L. Network abnormalities and interneuron dysfunction in Alzheimer disease. Nat Rev Neurosci. 2016 December; 17(12):777-792. doi: 10.1038/nrn.2016.141. Epub 2016 Nov. 10. Review.
Rudy B, McBain C J. Kv3 channels: voltage-gated K+ channels designed for high-frequency repetitive firing. Trends Neurosci. 2001 September; 24(9):517-26. Review.
Straub R E, Lipska B K, Egan M F, Goldberg T E, Callicott J H, Mayhew M B, Vakkalanka R K, Kolachana B S, Kleinman J E, Weinberger D R. Allelic variation in GAD1 (GAD67) is associated with schizophrenia and influences cortical function and gene expression. Mol Psychiatry. 2007 September; 12(9):854-69. Epub 2007 May 1.
Strumbos J G, Brown M R, Kronengold J, Polley D B, Kaczmarek L K. Fragile X mental retardation protein is required for rapid experience-dependent regulation of the potassium channel Kv3.1b. J Neurosci. 2010 Aug. 4; 30(31):10263-71. doi: 10.1523/JNEUROSCI.1125-10.2010.
Tsantoulas C, McMahon S B. Opening paths to novel analgesics: the role of potassium channels in chronic pain. Trends Neurosci. 2014 March; 37(3):146-58. doi: 10.1016/j.tins.2013.12.002. Epub 2014 Jan. 21. Review.
Veit J, Hakim R, Jadi M P, Sejnowski T J, Adesnik H. Cortical gamma band synchronization through somatostatin interneurons. Nat Neurosci. 2017 July; 20(7):951-959. doi: 10.1038/nn.4562. Epub 2017 May 8.
von Hehn C A, Bhattacharjee A, Kaczmarek L K. Loss of Kv3.1 tonotopicity and alterations in cAMP response element-binding protein signaling in central auditory neurons of hearing impaired mice. J Neurosci. 2004 Feb. 25; 24(8):1936-40.
Weiser M, Vega-Saenz de Miera E, Kentros C, Moreno H, Franzen L, Hillman D, Baker H, Rudy B. Differential expression of Shaw-related K+ channels in the rat central nervous system. J Neurosci. 1994 March; 14(3 Pt 1):949-72.
Alberico S L, Kim Y C, Lence T, Narayanan N S. Axial levodopa-induced dyskinesias and neuronal activity in the dorsal striatum. Neuroscience. 2017 Feb. 20; 343:240-249. doi: 10.1016/j.neuroscience.2016.11.046.
Burguière E, Monteiro P, Feng G, Graybiel A M. Optogenetic stimulation of lateral orbitofronto-striatal pathway suppresses compulsive behaviors. Science. 2013 Jun. 7; 340(6137):1243-6. doi:10.1126/science. 1232380.
Gittis A H, Leventhal D K, Fensterheim B A, Pettibone J R, Berke J D, Kreitzer A C. Selective inhibition of striatal fast-spiking interneurons causes dyskinesias. J Neurosci. 2011 Nov. 2; 31(44):15727-31.
Kataoka Y, Kalanithi P S, Grantz H, Schwartz M L, Saper C, Leckman J F, Vaccarino F M. Decreased number of parvalbumin and cholinergic interneurons in the striatum of individuals with Tourette syndrome. J Comp Neurol. 2010 Feb. 1; 518(3):277-91.
Kalanithi P S, Zheng W, Kataoka Y, DiFiglia M, Grantz H, Saper C B, Schwartz M L, Leckman J F, Vaccarino F M. Altered parvalbumin-positive neuron distribution in basal ganglia of individuals with Tourette syndrome. Proc Natl Acad Sci USA. 2005 Sep. 13; 102(37):13307-12.
Lallani S B, Villalba R M, Chen Y, Smith Y, Chan A W S. Striatal Interneurons inTransgenic Nonhuman Primate Model of Huntington's Disease. Sci Rep. 2019 Mar. 5; 9(1):3528.
Muñoz-Manchado A B, Bengtsson Gonzales C, Zeisel A, Munguba H, Bekkouche B, Skene N G, Lönnerberg P, Ryge J, Harris K D, Linnarsson S, Hjerling-Leffler J. Diversity of Interneurons in the Dorsal Striatum Revealed by Single-Cell RNA Sequencing and PatchSeq. Cell Rep. 2018 Aug. 21; 24(8):2179-2190.
Reiner A, Shelby E, Wang H, Demarch Z, Deng Y, Guley N H, Hogg V, Roxburgh R, Tippett L J, Waldvogel H J, Faull R L. Striatal parvalbuminergic neurons are lost in Huntington's disease: implications for dystonia. Mov Disord. 2013 October; 28(12):1691-9.

1. A compound of Formula I 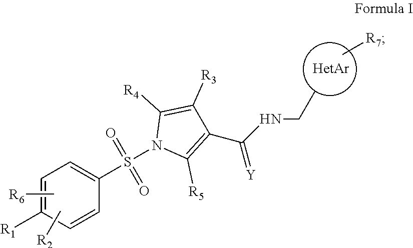
wherein
R₁ is selected from the group consisting of H, C₁-C₄ alkyl, C₁-C₄ fluoroalkyl, C₁-C₄ alkoxy, C₁-C₄ fluoroalkoxy, C₃-C₈ cycloalkyl, C₁-C₄ thioalkyl, C₁-C₄ thiofluoroalkyl, and halogen;
R₂ and R₆ are independently selected from the group consisting of H, C₁-C₄ alkyl, C₁-C₄ alkoxy, and halogen;
R₃ is selected from the group consisting of H, fluorine, and C₁-C₄ alkyl;
R₄ and R₅ are independently selected from the group consisting of H and fluorine;
R₇ is selected from the group consisting of H, C₁-C₄ alkyl, halogen, C₁-C₄ alkoxy, fluoroalkyl, fluoroalkoxy, and C₁-C₄ alkylamino;
Y is selected from the group consisting of oxygen and sulfur; and
HetAr is selected from the group consisting of 5-membered heteroaryl, 6-membered heteroaryl, and bicyclic heteroaromatic ring system, wherein HetAr may be optionally substituted with one or more independently selected R₇ substituents;
wherein when R₁ is C₁-C₄ alkoxy it may form a ring closure with R₂ or R₆ when any one of these is C₁-C₄ alkyl;
or a pharmaceutically acceptable salt thereof.

2. The compound according to claim 1, wherein R₁ is selected from the group consisting of hydrogen, methyl, difluoromethyl, trifluoromethyl, fluorine, chlorine, and methoxy.

3. The compound according to claim 1, wherein R₂ and R₆ are independently selected from the group consisting of hydrogen, fluorine, chlorine, bromine, methoxy, and methyl.

4. The compound according to claim 1, wherein R₃ is selected from the group consisting of hydrogen and methyl.

5. The compound according to claim 1, wherein R₄ and R₅ are independently selected from the group consisting of hydrogen, methyl, and fluorine.

6. The compound according to claim 1, wherein R₇ is selected from the group consisting of hydrogen, chlorine, fluorine, methyl, methoxy, and methylamino.

7. The compound according to claim 1, wherein HetAr is selected from the group consisting of pyrimidinyl, pyridazinyl, pyrazinyl, pyrazolyl, pyridyl, oxadiazolyl, isoxazolyl, oxazolyl, thiazolyl, imidazolyl, triazolyl, thiadiazolyl, and imidazopyrimidinyl.

8. The compound according to claim 1, wherein Y is oxygen.

9. The compound according to claim 1, wherein the compound is selected from the group consisting of
N-[(5-methylpyrimidin-2-yl)methyl]-1-(p-tolylsulfonyl)pyrrole-3-carboxamide,
N-[(2-methylpyrimidin-5-yl)methyl]-1-(p-tolylsulfonyl)pyrrole-3-carboxamide,
N-[(6-methylpyridazin-3-yl)methyl]-1-(p-tolylsulfonyl)pyrrole-3-carboxamide,
1-(2-fluorophenyl)sulfonyl-N-[(5-methylpyrazin-2-yl)methyl]pyrrole-3-carboxamide,
1-(3-fluorophenyl)sulfonyl-N-[(5-methylpyrazin-2-yl)methyl]pyrrole-3-carboxamide,
1-(4-fluorophenyl)sulfonyl-N-[(5-methylpyrazin-2-yl)methyl]pyrrole-3-carboxamide,
1-(4-methoxyphenyl)sulfonyl-N-[(5-methylpyrazin-2-yl)methyl]pyrrole-3-carboxamide,
4-methyl-N-[(5-methylpyrazin-2-yl)methyl]-1-(p-tolylsulfonyl)pyrrole-3-carboxamide,
1-(p-tolylsulfonyl)-N-(2-pyridylmethyl)pyrrole-3-carboxamide,
N-[(3-methoxy-2-pyridyl)methyl]-1-(p-tolylsulfonyl)pyrrole-3-carboxamide,
N-[(3-fluoro-2-pyridyl)methyl]-1-(p-tolylsulfonyl)pyrrole-3-carboxamide,
N-[(4-fluoro-2-pyridyl)methyl]-1-(p-tolylsulfonyl)pyrrole-3-carboxamide,
N-[(5-fluoro-2-pyridyl)methyl]-1-(p-tolylsulfonyl)pyrrole-3-carboxamide,
1-(p-tolylsulfonyl)-N-(3-pyridylmethyl)pyrrole-3-carboxamide,
N-[(6-methyl-2-pyridyl)methyl]-1-(p-tolylsulfonyl)pyrrole-3-carboxamide,
N-[(4-methyl-2-pyridyl)methyl]-1-(p-tolylsulfonyl)pyrrole-3-carboxamide,
N-[(3-methyl-2-pyridyl)methyl]-1-(p-tolylsulfonyl)pyrrole-3-carboxamide,
N-[(5-methoxy-2-pyridyl)methyl]-1-(p-tolylsulfonyl)pyrrole-3-carboxamide,
N-[(4-methoxy-2-pyridyl)methyl]-1-(p-tolylsulfonyl)pyrrole-3-carboxamide,
N-(imidazo[1,2-a]pyrimidin-6-ylmethyl)-1-(p-tolylsulfonyl)pyrrole-3-carboxamide,
N-[(5-methylpyrazin-2-yl)methyl]-1-(p-tolylsulfonyl)pyrrole-3-carboxamide,
N-[(6-methyl-3-pyridyl)methyl]-1-(p-tolylsulfonyl)pyrrole-3-carboxamide,
N-[(5-methyl-2-pyridyl)methyl]-1-(p-tolylsulfonyl)pyrrole-3-carboxamide,
N-[(5-methylpyrazin-2-yl)methyl]-1-(o-tolylsulfonyl)pyrrole-3-carboxamide,
1-(p-tolylsulfonyl)-N-(pyrazin-2-ylmethyl)pyrrole-3-carboxamide,
N-[(5-methylpyrazin-2-yl)methyl]-1-(m-tolylsulfonyl)pyrrole-3-carboxamide,
N-[(5-methyl-1,3,4-oxadiazol-2-yl)methyl]-1-(p-tolylsulfonyl)pyrrole-3-carboxamide,
N-[(5-methylisoxazol-3-yl)methyl]-1-(p-tolylsulfonyl)pyrrole-3-carboxamide,
N-[(5-methyl oxazol-2-yl)methyl]-1-(p-tolylsulfonyl)pyrrole-3-carboxamide,
N-[(4-methylthiazol-2-yl)methyl]-1-(p-tolyl sulfonyl)pyrrole-3-carboxamide,
N-[(3-methylisoxazol-5-yl)methyl]-1-(p-tolylsulfonyl)pyrrole-3-carboxamide,
N-[(1-methylpyrazol-3-yl)methyl]-1-(p-tolylsulfonyl)pyrrole-3-carboxamide,
N-[(1-methylpyrazol-4-yl)methyl]-1-(p-tolylsulfonyl)pyrrole-3-carboxamide,
N-[(2-methyloxazol-5-yl)methyl]-1-(p-tolylsulfonyl)pyrrole-3-carboxamide,
N-[(5-methylthiazol-2-yl)methyl]-1-(p-tolyl sulfonyl)pyrrole-3-carboxamide,
N-[(1-methylimidazol-4-yl)methyl]-1-(p-tolylsulfonyl)pyrrole-3-carboxamide,
N-[(1-methyltriazol-4-yl)methyl]-1-(p-tolylsulfonyl)pyrrole-3-carboxamide,
N-[(1-methyl-1,2,4-triazol-3-yl)methyl]-1-(p-tolylsulfonyl)pyrrole-3-carboxamide,
N-[(3-methyl-1,2,4-oxadiazol-5-yl)methyl]-1-(p-tolylsulfonyl)pyrrole-3-carboxamide,
1-(4-methylbenzene-1-sulfonyl)-N-[(2-methyl-1,3-oxazol-4-yl)methyl]-1H-pyrrole-3-carboxamide,
1-(benzenesulfonyl)-N-[(5-methylpyrazin-2-yl)methyl]-1H-pyrrole-3-carboxamide,
1-(4-methylbenzene-1-sulfonyl)-N-[(1,3-thiazol-4-yl)methyl]-1H-pyrrole-3-carboxamide,
1-(4-methylbenzene-1-sulfonyl)-N-[(1,3-oxazol-5-yl)methyl]-1H-pyrrole-3-carboxamide,
1-(4-methylbenzene-1-sulfonyl)-N-[(1,3-thiazol-2-yl)methyl]-1H-pyrrole-3-carboxamide,
1-(4-methylbenzene-1-sulfonyl)-N-[(1,2-oxazol-3-yl)methyl]-1H-pyrrole-3-carboxamide,
1-(4-methylbenzene-1-sulfonyl)-N-[(1,2-oxazol-5-yl)methyl]-1H-pyrrole-3-carboxamide,
1-(4-methylbenzene-1-sulfonyl)-N-[(1,3-oxazol-4-yl)methyl]-1H-pyrrole-3-carboxamide,
1-(4-methylbenzene-1-sulfonyl)-N-[(1,2-thiazol-4-yl)methyl]-1H-pyrrole-3-carboxamide,
1-(4-methylbenzene-1-sulfonyl)-N-[(1,3,4-thiadiazol-2-yl)methyl]-1H-pyrrole-3-carboxamide,
1-(4-methylbenzene-1-sulfonyl)-N-[(1,2,4-oxadiazol-3-yl)methyl]-1H-pyrrole-3-carboxamide,
1-(4-methylbenzene-1-sulfonyl)-N-[(pyrimidin-5-yl)methyl]-1H-pyrrole-3-carboxamide,
1-(2-fluorobenzene-1-sulfonyl)-N-[(pyrazin-2-yl)methyl]-1H-pyrrole-3-carboxamide,
1-(3-methylbenzene-1-sulfonyl)-N-[(1-methyl-1H-pyrazol-3-yl)methyl]-1H-pyrrole-3-carboxamide,
1-(3-methylbenzene-1-sulfonyl)-N-[(3-methyl-1,2,4-oxadiazol-5-yl)methyl]-1H-pyrrole-3-carboxamide,
1-(3-methylbenzene-1-sulfonyl)-N-[(5-methylpyrimidin-2-yl)methyl]-1H-pyrrole-3-carboxamide,
1-(4-fluorobenzene-1-sulfonyl)-N-[(1-methyl-1H-pyrazol-3-yl)methyl]-1H-pyrrole-3-carboxamide,
1-(4-fluorobenzene-1-sulfonyl)-N-[(pyrazin-2-yl)methyl]-1H-pyrrole-3-carboxamide,
1-(4-fluorobenzene-1-sulfonyl)-N-[(3-methyl-1,2,4-oxadiazol-5-yl)methyl]-1H-pyrrole-3-carboxamide,
1-(4-methoxybenzene-1-sulfonyl)-N-[(1-methyl-1H-pyrazol-3-yl)methyl]-1H-pyrrole-3-carboxamide,
1-(4-methoxybenzene-1-sulfonyl)-N-[(pyrazin-2-yl)methyl]-1H-pyrrole-3-carboxamide,
1-(4-methoxybenzene-1-sulfonyl)-N-[(3-methyl-1,2,4-oxadiazol-5-yl)methyl]-1H-pyrrole-3-carboxamide,
1-(4-methoxybenzene-1-sulfonyl)-N-[(1-methyl-1H-pyrazol-4-yl)methyl]-1H-pyrrole-3-carboxamide,
1-(4-methoxybenzene-1-sulfonyl)-N-[(5-methylpyridin-2-yl)methyl]-1H-pyrrole-3-carboxamide,
1-(4-methoxybenzene-1-sulfonyl)-N-[(3-methyl-1,2-oxazol-5-yl)methyl]-1H-pyrrole-3-carboxamide,
1-(4-methoxybenzene-1-sulfonyl)-N-[(5-methylpyrimidin-2-yl)methyl]-1H-pyrrole-3-carboxamide,
1-(4-methoxybenzene-1-sulfonyl)-N-[(5-methyl-1,3-oxazol-2-yl)methyl]-1H-pyrrole-3-carboxamide,
1-(2-methylbenzene-1-sulfonyl)-N-[(1-methyl-1H-pyrazol-3-yl)methyl]-1H-pyrrole-3-carboxamide,
1-(2-methylbenzene-1-sulfonyl)-N-[(3-methyl-1,2,4-oxadiazol-5-yl)methyl]-1H-pyrrole-3-carboxamide,
1-(2-methylbenzene-1-sulfonyl)-N-[(1-methyl-1H-pyrazol-4-yl)methyl]-1H-pyrrole-3-carboxamide,
1-(2-methylbenzene-1-sulfonyl)-N-[(5-methylpyridin-2-yl)methyl]-1H-pyrrole-3-carboxamide,
1-(2-methylbenzene-1-sulfonyl)-N-[(3-methyl-1,2-oxazol-5-yl)methyl]-1H-pyrrole-3-carboxamide,
1-(2-methylbenzene-1-sulfonyl)-N-[(5-methyl-1,3-oxazol-2-yl)methyl]-1H-pyrrole-3-carboxamide,
1-(4-chlorobenzene-1-sulfonyl)-N-[(pyrazin-2-yl)methyl]-1H-pyrrole-3-carboxamide,
1-(benzenesulfonyl)-N-[(1-methyl-1H-pyrazol-3-yl)methyl]-1H-pyrrole-3-carboxamide,
1-(benzenesulfonyl)-N-[(5-methylpyridin-2-yl)methyl]-1H-pyrrole-3-carboxamide,
1-(benzenesulfonyl)-N-[(3-methyl-1,2-oxazol-5-yl)methyl]-1H-pyrrole-3-carboxamide,
1-(4-fluorobenzene-1-sulfonyl)-N-[(6-methylpyridin-3-yl)methyl]-1H-pyrrole-3-carboxamide,
1-(4-methylbenzene-1-sulfonyl)-N-[(1,3-oxazol-2-yl)methyl]-1H-pyrrole-3-carboxamide,
5-fluoro-1-(4-methylbenzene-1-sulfonyl)-N-[(5-methylpyrazin-2-yl)methyl]-1H-pyrrole-3-carboxamide,
2-fluoro-1-(4-methylbenzene-1-sulfonyl)-N-[(5-methylpyrazin-2-yl)methyl]-1H-pyrrole-3-carboxamide,
N-[(5-chloropyrazin-2-yl)methyl]-1-(4-methylbenzene-1-sulfonyl)-1H-pyrrole-3-carboxamide,
1-(4-fluoro-2-methylbenzene-1-sulfonyl)-N-[(5-methylpyrazin-2-yl)methyl]-1H-pyrrole-3-carboxamide,
N-[(5-methylpyrazin-2-yl)methyl]-1-[4-(trifluoromethyl)benzene-1-sulfonyl]-1H-pyrrole-3-carboxamide,
1-(3-chloro-4-fluorobenzene-1-sulfonyl)-N-[(5-methylpyrazin-2-yl)methyl]-1H-pyrrole-3-carboxamide,
1-[4-(difluoromethyl)benzene-1-sulfonyl]-N-[(5-methylpyrazin-2-yl)methyl]-1H-pyrrole-3-carboxamide,
1-(4-methylbenzene-1-sulfonyl)-N-[(5-methylpyrazin-2-yl)methyl]-1H-pyrrole-3-carbothioamide,
1-(2-fluoro-4-methyl-phenyl)sulfonyl-N-[(5-methylpyrazin-2-yl)methylpyrrole-3-carboxamide,
1-(2-fluoro-4-methoxy-phenyl)sulfonyl-N-[(5-methylpyrazin-2-yl)methyl]pyrrole-3-carboxamide,
1-(3-fluoro-4-methoxy-phenyl)sulfonyl-N-(5-methylpyrazin-2-yl)methyl]pyrrole-3-carboxamide,
1-(4-methoxy-2-methyl-phenyl)sulfonyl-N-[(5-methylpyrazin-2-yl)methyl]pyrrole-3-carboxamide,
1-(4-fluoro-2,6-dimethyl-phenyl)sulfonyl-N-[(5-methylpyrazin-2-yl)methyl]pyrrole-3-carboxamide,
1-(4-fluoro-3,5-dimethyl-phenyl)sulfonyl-N-[(5-methylpyrazin-2-yl)methyl]pyrrole-3-carboxamide,
1-(4-fluoro-3-methyl-phenyl)sulfonyl-N-[(5-methylpyrazin-2-yl)methyl]pyrrole-3-carboxamide,
1-(2,3-dihydrobenzofuran-5-ylsulfonyl)-N-[(5-methylpyrazin-2-yl)methyl]pyrrole-3-carboxamide,
N-[(5-methylpyrazin-2-yl)methyl]-1-(2,4,6-trimethylphenyl)sulfonyl-pyrrole-3-carboxamide,
1-(2-chloro-4-methoxy-phenyl)sulfonyl-N-[(5-methylpyrazin-2-yl)methyl]pyrrole-3-carboxamide,
1-(2-bromo-4-methoxy-phenyl)sulfonyl-N-[(5-methylpyrazin-2-yl)methyl]pyrrole-3-carboxamide,
1-(2-fluoro-4-m ethylbenzene-1-sulfonyl)-N-{[5-(methylamino)pyrazin-2-yl]methyl}-1H-pyrrole-3-carboxamide,
1-[4-(difluoromethoxy)benzene-1-sulfonyl]-N-{[5-methylamino)pyran-2-yl]methyl}-1H-pyrrole-3-carboxamide,
1-(2-fluoro-4-methylbenzene-1-sulfonyl)-N-[(2-methylpyrimidin-5-yl)methyl]-1H-pyrrole-3-carboxamide,
1-(4-methylbenzene-1-sulfonyl)-N-[(2-methyl-2H-1,2,3-triazol-4-yl)methyl]-1H-pyrrole-3-carboxamide,
1-(2-fluoro-4-methylbenzene-1-sulfonyl)-N-[(2-methoxypyrimidin-5-yl)methyl]-1H-pyrrole-3-carboxamide,
1-(benzenesulfonyl)-N-[(3,5-dimethylpyrazin-2-yl)methyl]-1H-pyrrole-3-carboxamide,
1-[4-(difluoromethoxy)benzene-1-sulfonyl]-N-[(2-methoxypyrimidin-5-yl)methyl]-1H-pyrrole-3-carboxamide,
1-benzenesulfonyl)-N-[(3-chloro-5-methylpyrazin-2-yl)methyl]-1H-pyrrole-3-carboxamide,
1-(4-methylbenzene-1-sulfonyl)-N-[(2-methyl-1,3-thiazol-5-yl)methyl]-1H-pyrrole-3-carboxamide,
1-(4-methylbenzene-1-sulfonyl)-N-[(5-methyl-1,3,4-thiadiazol-2-yl)methyl]-1H-pyrrole-3-carboxamide,
1-(4-methylbenzene-1-sulfonyl)-N-[(3-methyl-1H-pyrazol-5-yl)methyl]-1H-pyrrole-3-carboxamide,
1-(2-chloro-4-methoxybenzene-1-sulfonyl)-N-[(1-methyl-1H-pyrazol-3-yl)methyl]-1H-pyrrole-3-carboxamide, and
1-(2-chloro-4-methoxybenzene-1-sulfonyl)-N-[(5-methylpyrimidin-2-yl)methyl]-1H-pyrrole-3-carboxamide.

10. A pharmaceutical composition comprising the compound according to claim 1 and one or more pharmaceutically acceptable excipients.

11. (canceled)

12. (canceled)

13. A method for the treatment of a neurological or psychiatric disorder comprising the administration of a therapeutically effective amount of the compound according to claim 1, or the pharmaceutical composition of claim 10, to a patient in need thereof.

14. (canceled)

15. The method of claim 13, wherein the neurological or psychiatric disorder is selected from the group consisting of epilepsy, schizophrenia, schizophreniform disorder, schizoaffective disorder, cognitive impairment associated with schizophrenia (CIAS), autism spectrum disorder, bipolar disorder, attention deficit hyperactivity disorder (ADHD), anxiety-related disorders, depression, cognitive dysfunction, Alzheimer's disease, fragile X syndrome, chronic pain, hearing loss, sleep and circadian disorders, sleep disruption and movement disorders, Huntington's disease, L-dopa-induced dyskinesia, obsessive-compulsive disorders, and Tourette's syndrome.

16. (canceled)

17. (canceled)

18. The method of claim 15, wherein the schizophrenia is of the paranoid, disorganized, catatonic, undifferentiated, or residual type.

19. The method of claim 15, wherein the schizoaffective disorder is of the delusional or depressive type.

20. The compound of claim 1, wherein
R₁ is hydrogen, methyl, difluoromethyl, trifluoromethyl, fluorine, chlorine, or methoxy;
R₂ and R₆ are independently hydrogen, fluorine, chlorine, bromine, methoxy, or methyl;
R₃ is hydrogen or methyl;
R₄ and R₅ are independently hydrogen, methyl or fluorine;
R₇ is hydrogen, chlorine, fluorine, methyl, methoxy or methylamino;
HetAr is pyrimidinyl, pyridazinyl, pyrazinyl, pyrazolyl, pyridyl, oxadiazolyl, isoxazolyl, oxazolyl, thiazolyl, imadozolyl, triazolyl, thiadiazolyl, or imidazopyrimidinyl; and
Y is oxygen.
